(19) United States
(12) Patent Application Publication
(10) Pub. No.: US 2008/0318218 A1
(43) Pub. Date: Dec. 25, 2008

(54) Compositions and Methods for Inferring an Adverse Effect in Response to a Drug Treatment

(75) Inventor: Tony N. Frudakis, Bradenton, FL (US)

Correspondence Address: DLA PIPER LLP (US), 4365 EXECUTIVE DRIVE, SUITE 1100, SAN DIEGO, CA, 92121-2133, US

(73) Assignee: DNAPrint Genomics, Inc.

(21) Appl. No.: 11/667,532

(22) PCT Filed: Nov. 14, 2005
(86) PCT No.: PCT/US05/41326

§ 371 Date: Nov. 15, 2007

Related U.S. Application Data
(60) Provisional application No. 60/627,453, filed on Nov. 12, 2004.

(51) Int. Cl. C12Q 1/68

(52) U.S. Cl. 435/6

(57) ABSTRACT

Methods are provided for inferring whether a subject to be treated with a drug such as a statin or an ACE inhibitor is likely to suffer an adverse effect due to the treatment. Also provided are compositions for practicing the methods.

### BACKGROUND OF THE INVENTION

1. Field of the Invention

The invention relates generally to methods for inferring a muscle adverse effect due to treatment with a statin, and more specifically to methods of detecting single nucleotide polymorphisms and combinations thereof in a nucleic acid sample that provide an inference as to whether is likely or not likely to have a muscle adverse effect in response to treatment with a statin.

2. Background Information

Heart attacks are the leading cause of death in the United States today. An increased risk of heart attack is linked with abnormally high blood cholesterol levels. Patients with abnormally high cholesterol levels are frequently prescribed a class of drugs called statins to reduce cholesterol levels, thereby reducing the risk of heart attack. However, these drugs are not effective in all patients. Furthermore, in some patients, adverse reactions including muscle adverse effect (e.g., myopathy and rhabdomyolysis). Such an adverse response may require that treatment of a patient be changed or discontinued.

Variable statin responses likely can be explained, at least in part, by genetic differences of patients. Human beings differ by up to 0.1% of the 3 billion letters of DNA present in the human genome. Though humans are 99.9% identical in genetic sequence, it is the 0.1% that determines the uniqueness of an individual. Though our individuality is apparent from visual inspection—anyone can recognize that we have facial features, heights and colors, and that these features are, to an extent, heritable (i.e. sons and daughters tend to resemble their parents more than strangers)-our individuality extends to less apparent features, including the ability to respond to and metabolize drugs.

An identification of the precise molecule details responsible for individuality is a challenging task. The human genome project resulted in the sequencing of the human genome. However, this sequencing was the result of sampling taken from a small number of individuals. Therefore, while this sequencing was an important scientific milestone, the initial sequencing of the human genome does not provide adequate information regarding genetic differences between individuals to allow identification of markers on the genome that are responsible for our individuality, particularly whether an individual will respond to statins and, if so, whether adverse effects such as muscle adverse effects are likely to occur. If the genetic markers that were responsible for different statin responses between people were identified, then an individual's genotype for key markers could be determined, and this information could be used by a physician to decide whether to prescribe statins, and which statins to prescribe. Such personalized medicine can improve the response rate in individuals, while, at the same time, reducing adverse reactions. Thus, there is a need for methods and compositions that allow an inference of muscle adverse effects in a subject treated with as statin based on an individual's genotype for key markers.

### SUMMARY OF THE INVENTION

The present invention is based, in part, on the identification of single nucleotide polymorphisms (SNPs) that are associated with adverse effects due to drug treatment. As such, the SNPs provide a tool for personalized medicine in that the identification of one or more SNPs allows an inference to be drawn as to whether a human subject to be treated with a drug, particularly a statin or an angiotensin converting enzyme (ACE) inhibitor is likely to suffer an adverse effect due to treatment with the drug.

Accordingly, in one embodiment, the present invention relates to a method for inferring a muscle adverse effect statin response of a human subject from a nucleic acid sample of the subject. Such a method can be performed, for example, by identifying, in a nucleic acid sample from the subject, a nucleotide occurrence of at least one statin response-related SNP of a marker as set forth in any of Tables 1, 2, 4, 5, or a combination thereof, whereby the nucleotide occurrence is associated with a muscle adverse effect in response to administration of the statin, thereby inferring the muscle adverse effect statin response of the subject. The muscle adverse effect statin response, which can be detected by a patient describing the symptoms or by measuring creatine kinase (CK) levels in the subject, can be any undesirable muscle or musculoskeletal effect, and can include symptoms varying from mild aching to severe pain, usually in proximal muscle groups, muscle stiffness and weakness, myalgia (usually associated with a 3-10 fold increase in CK levels above normal), myopathy (usually associated with CK levels more than 10 times greater than normal), or rhabdomyolysis (usually associated with CK levels more than 40 times the upper limit of normal).

In certain embodiments, the marker is nucleotides 3911-4379 of SEQ ID NO: 134; nucleotides 31264-31822 of SEQ ID NO: 131; nucleotides 22281-23778 of SEQ ID NO: 143; nucleotides 3482-4414 of SEQ ID NO: 134; e. nucleotides 1895-2286 of SEQ ID NO: 132; nucleotides 650-1166 of SEQ ID NO: 131; nucleotides 771-1171 of SEQ ID NO: 142; nucleotides 79999-80360 of SEQ ID NO: 133; nucleotides 31264-31822 of SEQ ID NO: 131; nucleotides 2440-2560 of SEQ ID NO: 134; nucleotides 23757-24069 of SEQ ID NO: 131; nucleotides 30438-30711 of SEQ ID NO: 131; nucleotides 23571-24967 of SEQ ID NO: 131; nucleotides 12971-14510 of SEQ ID NO: 143; nucleotides 26896-27098 of SEQ ID NO: 130; nucleotides 8115-8737 of SEQ ID NO: 143; nucleotides 13465-13865 of SEQ ID NO:138; nucleotides 26056-26456 of SEQ ID NO: 138; nucleotides 26167-26197 of SEQ ID NO: 138; nucleotides 17636-18035 of SEQ ID NO: 138; nucleotides 25354-25754 of SEQ ID NO: 138; nucleotides 12153-12553 of SEQ ID NO:138; nucleotides 7082-7942 of SEQ ID NO:139; nucleotides 5779-5827 of SEQ ID NO:135; nucleotides 5851-6442 of SEQ ID NO:135; nucleotides 7909-8504 of SEQ ID NO: 139; nucleotides 651-1166 of SEQ ID NO: 131; nucleotides 4351-4750 of SEQ ID NO:139 nucleotides 3138-3500 of SEQ ID NO:139; nucleotides 3482-4414 of SEQ ID NO:131; nucleotides 4397-4797 of SEQ ID NO:139; nucleotides 31264-31813 of SEQ ID NO:131; nucleotides 16240-16589 of SEQ ID NO:145; nucleotides 25192-2298 of SEQ ID NO:145; nucleotides 11344-12528 of SEQ ID NO:139; nucleotides 2800-3685 of SEQ ID NO:129; 30350-30631 of SEQ ID NO:131; nucleotides 750-1110 of SEQ ID NO:134; nucleotides 5880-6229 of SEQ ID NO:145; nucleotides 25192-25479 of SEQ ID NO:145; nucleotides 17794-18106 of SEQ ID NO:130; nucleotides 26895-27098 of SEQ ID NO: 130; nucleotides 26895-25478 of SEQ ID NO: 130; nucleotides 34-825 of SEQ ID NO: 127; nucleotides 11012-11412 of SEQ ID NO: 135; nucleotides 3178-3786 of SEQ ID NO:134; nucleotides 143-518 of SEQ ID NO:140; nucleotides 17795-18116 of SEQ ID NO:130; nucleotides 3388-3786 of SEQ ID NO:134; nucleotides 502-902 of SEQ ID NO:126; nucleotides 23737-24368 of SEQ ID NO:131; nucleotides 1805-2204 of SEQ ID NO:131; nucleotides 5841-6441 of SEQ ID NO:135; nucleotides 26613-27098 of SEQ ID NO:130; nucleotides 19968-20369 of SEQ ID NO:138; nucleotides 19636-21357 of SEQ ID NO: 136; nucleotides 2440-2560 of SEQ ID NO: 134; nucleotides 5881-6229 of SEQ ID NO: 142; the complement of any of these nucleotide regions.

In various aspects of the invention, the SNP is located at nucleotide 4332 of SEQ ID NO: 134; nucleotide 31683 of SEQ ID NO: 131; nucleotide 23077 of SEQ ID NO: 143; nucleotide 4208 of SEQ ID NO: 134; nucleotide 2098 of SEQ ID NO: 132; nucleotide 860 of SEQ ID NO:131; nucleotide 971 of SEQ ID NO:142; nucleotide 2098 of SEQ ID NO: 133; nucleotide 80160 SEQ ID NO: 131; nucleotide 2500 of SEQ ID NO: 134; nucleotide 23809 of SEQ ID NO: 131; nucleotide 30635 of SEQ ID NO: 131; nucleotide 24272 of SEQ ID NO: 131; nucleotide 13780 of SEQ ID NO: 143; nucleotide 296935 of SEQ ID NO: 130; nucleotide 8462 of SEQ ID NO: 143; nucleotide 13665 of SEQ ID NO:138; nucleotide 26256 of SEQ ID NO:138; nucleotide 26137 of SEQ ID NO:138; nucleotide 17836 of SEQ ID NO:138; nucleotide 25554 of SEQ ID NO:138; nucleotide 12353 of SEQ ID NO:138; nucleotide 7444 of SEQ ID NO:139; nucleotide 5832 of SEQ ID NO:135; nucleotide 6063 of SEQ ID NO:135; nucleotide 8004 of SEQ ID NO:139; nucleotide 860 of SEQ ID NO:131; nucleotide 4550 of SEQ ID NO:139; nucleotide 3300 of SEQ ID NO:139; nucleotide 4208 of SEQ ID NO:131; nucleotide 4597 of SEQ ID NO:139; nucleotide 31671 of SEQ ID NO:131; nucleotide 16399 of SEQ ID NO: 145; nucleotide 2097 of SEQ ID NO: 145; nucleotide 11987 of SEQ ID NO: 139; nucleotide 3500 of SEQ ID NO:129; nucleotide 30434 of SEQ ID NO:131; nucleotide 930 of SEQ ID NO:134; nucleotide 6046 of SEQ ID NO:145; nucleotide 25286 of SEQ ID NO: 145; nucleotide 18060 of SEQ ID NO:130; nucleotide 26950 of SEQ ID NO:130; nucleotide 26950 of SEQ ID NO:130; nucleotide 734 of SEQ ID NO:127; nucleotide 11212 of SEQ ID NO:135; nucleotide 3671 of SEQ ID NO:134; nucleotide 326 of SEQ ID NO:140; nucleotide 18060 of SEQ ID NO:130; nucleotide 3671 of SEQ ID NO: 134; nucleotide 702 of SEQ ID NO:126; nucleotide 24205 of SEQ ID NO:131; nucleotide 2005 of SEQ ID NO:131; nucleotide 6063 of SEQ ID NO:135; nucleotide 26806 of SEQ ID NO: 130; nucleotide 20169 of SEQ ID NO: 138; nucleotide 20343 of SEQ ID NO: 136; nucleotide 6183 of SEQ ID NO: 142 or nucleotide 2500 of SEQ ID NO: 134.

A method of identifying a nucleotide occurrence of at least one statin response-related SNP can be performed, for example, by incubating the nucleic acid sample with a probe or primer that selectively hybridizes to or near a nucleic acid molecule comprising the nucleotide occurrence of the SNP, and detecting selective hybridization of the primer or probe. Selective hybridization of the primer can be detected, for example, by performing a primer extension reaction, and detecting a primer extension reaction product comprising the primer. In one aspect, the primer extension reaction comprises a polymerase chain reaction.

In another aspect, the method includes identifying a nucleotide occurrence of each of at least two statin response-related SNPs. The statin response-related SNP can be a SNP as set forth in any of SEQ ID NOS:1-90, or a combination thereof. For example, the statin response-related SNP can be a Lipitor® statin response-related SNP as set forth in any of SEQ ID NOS:1-45, or a combination thereof, the detection of such. SNPs being useful for determining whether a subject should be treated with a Lipitor® statin; or can be a Zocor® statin response-related SNP as set forth in any of SEQ ID NOS:28, 32, 38, 41, 43, and 46-90, or a combination thereof, the detection of such SNPs being useful for determining whether a subject should be treated with a Zocor® statin.

In another embodiment, the present invention relates to a method for inferring a dry cough adverse effect ACE inhibitor response of a human subject from a nucleic acid sample of the subject. Such a method can be performed, for example, by identifying, in the nucleic acid sample, a nucleotide occurrence of at least one ACE inhibitor response-related SNP of a marker as set forth in any of Tables 1, 2, 4, 5, or a combination thereof, whereby the nucleotide occurrence is associated with a dry cough effect in response to administration of the ACE inhibitor, thereby inferring the dry cough adverse effect ACE inhibitor response of the subject. The ACE inhibitor can be any ACE inhibitor commonly used to treat high blood pressure, cardiovascular disease, and the like, including, for example, benazepril (Lotensin®; Novartis), captopril (Capoten®; Bristol-Myers Squibb), enalapril (Vasotec®; Merck), fosinopril (Monopril®; Bristol-Myers Squibb), and lisinopril—Prinivil®; Merck; also Zestril®; Astra-Zeneca).

A method of identifying a nucleotide occurrence of at least one ACE inhibitor response-related SNP can be performed, for example, by incubating the nucleic acid sample with a probe or primer that selectively hybridizes to or near a nucleic acid molecule comprising the nucleotide occurrence of the SNP, and detecting selective hybridization of the primer or probe, thereby identifying the nucleotide occurrence. Selective hybridization of the primer can be performed as discussed above, including, for example, by detecting a primer extension reaction product comprising the primer, wherein the primer extension reaction can be a polymerase chain reaction.

In one aspect, the method is performed by identifying a nucleotide occurrence of each of at least two ACE inhibitor response-related SNPs. The ACE inhibitor response-related SNP identified according to the present methods can be one or more SNPs as set forth in any of SEQ ID NOS:91-124. For example, the ACE inhibitor response-related SNP can be an enalapril ACE inhibitor response-related SNP as set forth in any of SEQ ID NOS:91-109, or a combination thereof, the detection of such SNPs being useful for determining whether a subject should be treated with enapril; or can be a lisinopril ACE inhibitor response-related SNP as set forth in any of SEQ ID NOS:110-124, or a combination thereof, the detection of such SNPs being useful for determining whether a subject should be treated with normal doses of lisinopril.

A method for inferring a poor metabolizer phenotype of a human subject from a nucleic acid sample of the subject is also provided by the invention. The method comprises identifying in the nucleic acid sample, an occurrence of at least one single nucleotide polymorphism (SNP) of a CYP2D6 marker, wherein the SNP is associated with the poor metabolizer phenotype, thereby inferring the poor metabolizer phenotype of the subject. In one embodiment, the poor metabolizer phenotype is associated with the CYP2D6*4 allele.

In certain aspects of this method the CYP2D6 marker comprises at least about 100 nucleotides of SEQ ID NO: 134, 147 or 148. The marker can for example, be nucleotides 3911-4379 of SEQ ID NO:134 and the SNP can be located at nucleotide 4332 of SEQ ID NO: 134. In other aspects of the invention, the marker is nucleotides 2440-2560 of SEQ ID NO: 134 while the SNP can be located at nucleotide 2500 of SEQ ID NO:134. In one embodiment SNPs in both markers are detected, which can involve comprising identifying the genotype of the SNPs at nucleotide 4332 of SEQ ID NO:134 and nucleotide 2500 of SEQ ID NO:134, wherein a TT and TC genotype or a TC and TC genotype, respectively, is associated with a poor metabolizer phenotype.

The marker can, for example, be CYP2D6_; RS1058174 or CYP2D6_RS2267446. In one embodiment, the poor metabolizer phenotype is associated with myalgia in atorvastatin-treated patients. According to this method of the invention, a myalgia response to atorvostatin can be inferred.

The present invention also relates to compositions for practicing the present methods, including, for example, primers and probes useful for detecting a SNP as set forth in Tables 1, 2, 4 and 5, and/or in SEQ ID NOS:1 to 124. Also provided are kits, which can contain such compositions and are useful for practicing the present methods.

### DETAILED DESCRIPTION OF THE INVENTION

Statins such as Lipitor® and Zocor® are a popular class of drugs used to treat hypercholesterolemia and significantly reduce risk of cardiovascular disease. The statins are 2-hydroxy-3-methylglutaryl coenzyme A (HMG-CoA) inhibitors. Treatment with Lipitor® and Zocor®, as well as most other statins, has been associated with a muscle adverse reaction (myopathy) characterized by rhabdomyolysis, serum creatine kinase (CK) elevations, and muscle pain (myalgia), weakness and/or cramping. Statin-induced myopathy is rare; Thompson et al., (JAMA 289:1681-1690. (2003), the entire contents of which are incorporated by reference herein) described FDA MEDWATCH Report database search results suggesting that the incidence of myopathy is 1% to 5%. Myopathology is a serious side effect of statin treatment, and statin administration to patients experiencing myopathological symptoms is routinely discontinued, or the patients are switched to another drug. The pathology arises from an over-exuberant response to the statin such that synthetic cholesterol levels are suppressed to such a degree or in such a way that normal cellular functions requiring cholesterol are inhibited. One of these functions is the construction of bilayer lipid cellular membranes involved in natural cell turnover, and the first symptom of inhibition of cellular replenishment is manifest as muscle pain, particularly where physical action, cellular stress, and cell turnover is relatively extreme.

Fortunately, death due to myopathologic adverse responses to statin treatment is relatively rare, in part because physicians are aware of and sensitive to the problem. Thompson et al., supra, reported a death incidence due to rhabdomyolysis at 0.15 deaths per 1 million prescriptions. However, treating patients with drugs that cause adverse side effects is clearly undesirable, is not cost effective, and the long term consequences of even mild myopathology is not clearly understood. As such, it would be beneficial to be able to predict whether a patient is more likely than others to have an adverse muscle response to a given statin so that the drug can be avoided, thus eliminating wasted drug as well as minimizing the chances of causing damage that may very well have long-term, chronic effects later in life. Since both Lipitor® and Zocor® are metabolized by the cytochrome P450 system, and since aberrant metabolism of drugs is commonly associated with adverse events and reduced efficacy, a systematic candidate gene screen and a whole genome screen were initiated to identify markers associated with myopathologic adverse events for both Lipitor® and Zocor®.

As disclosed herein, SNPs have been identified that allow an inference to be drawn as to whether a human individual is likely to respond to treatment with a statin (Example 1) or with an angiotensin converting enzyme (ACE) inhibitor (Example 2), including whether the individual may have an adverse effect due to the treatment. Accordingly, in one embodiment, the invention relates to methods for inferring a statin response, including a muscle adverse effect, of a human subject from a nucleic acid sample of the subject. In another embodiment, the invention relates to methods for inferring an ACE inhibitor response, including a dry cough adverse effect, of a human subject from a nucleic acid sample of the subject.

The methods of the invention are based, in part, on the identification of single nucleotide polymorphisms (SNPs) that, alone or in combination, including when combined into haplotypes, allow an inference to be drawn as to a statin response or an ACE inhibitor response. As such, the compositions and methods of the invention are useful, for example, for identifying patients who are more likely than others to respond to statin (or ACE inhibitor) treatment and more likely than others not to suffer adverse effects of statin (or ACE inhibitor) treatment. In one aspect, the invention provides a method for inferring a statin response of a human subject from a nucleic acid sample of the subject by identifying in the biological sample, a nucleotide occurrence of at least one statin response-related single nucleotide polymorphism (SNP) corresponding to a SNP as set forth in Example 1. In another aspect, the invention provides a method for inferring an ACE inhibitor response of a human subject from a nucleic acid sample of the subject by identifying in the biological sample, a nucleotide occurrence of at least one ACE inhibitor response-related SNP corresponding to a SNP as set forth in Example 2. It will be recognized that the SNPs to be examined will depend on the particular statin (or ACE inhibitor) to be prescribed, such SNPs being identified in Examples 1 and 2. In another aspect of the invention, a nucleotide occurrence of each of at least two statin (or ACE inhibitor) response-related SNPs is identified, wherein the nucleotide occurrences of at least two of the statin (or ACE inhibitor) response-related SNPs can comprise at least one haplotype allele.

An inference drawn according to a method of the invention can be strengthened by identifying a second, third, fourth or more statin (or ACE inhibitor) response-related haplotype allele in the same, or preferably different statin (or ACE inhibitor) response-related gene(s). Accordingly, the method can further include identifying in the nucleic acid sample at least a second response-related haplotype allele.

Statins are a class of medications that have been shown to be effective in lowering human total cholesterol (TC) and low density lipoprotein (LDL) levels in hyperlipidemic patients. The drugs act at the step of cholesterol synthesis. By reducing the amount of cholesterol synthesized by the cell, through inhibition of the HMG CoA reductase gene (HMGCR), the drug initiates a cycle of events that culminates in the increase of LDL uptake by liver cells. As LDL uptake is increased, total cholesterol and LDL levels in the blood decrease. Lower blood levels of both factors are associated with lower risk of atherosclerosis and heart disease, and the Statins are widely used to reduce atherosclerotic morbidity and mortality. Nonetheless, some patients show no response to a given statin.

Methods of the present invention provide an inference of a statin response after administration of statins to a subject. The inference of the present invention assumes that statins are administered at an effective dosage, for example, using FDA approved guidelines including dosages, for those statins that are FDA approved. An effective dosage is a dosage where a statin has been shown to reduce serum cholesterol in the general population. It will be understood that any method of the present invention, or SNP identified herein, will be useful both for predicting a positive response to statins (or ACE inhibitors), as well as a negative response (e.g., a muscle adverse effect).

Drugs such as statins are referred to a “xenobiotics” because they are chemical compounds that are not naturally found in the human body. Xenobiotic metabolism genes make proteins whose sole purpose is to detoxify foreign compounds present in the human body, and they evolved to allow humans to degrade and excrete harmful chemicals present in many foods (such as tannins and alkaloids from which many drugs are derived). Examples of statins include, but are not limited to, Fluvastatin (Lescol™), Atorvastatin (Lipitor™), Lovastatin (Mevacor™), Pravastatin (Pravachol™), Simvastatin (Zocor™), Cerivastatin (Baycol™). The chemical structure of these statins are known and widely available. For example, Atorvastatin calcium is {R—(R*,R*)}-2-(4-fluorophenyl)-b,d-dihydroxy-5-(1-methylethyl)-3-phenyl-4 {(phenylamino)carbonyl}-1H-pyrrole-1-heptanoic acid, calcium salt (2:1) trihydrate. The empirical formula of atorvastatin calcium is (C₃₃H₃₄FN₂O₅)₂Ca.3H2O and its molecular weight is 1209.42. Simvastatin is butanoic acid, 2,2-dimethyl-, 1,2,3,7,8,8a-hexahydro-3,7-dimethyl-8-{2-(tetrahydro-4-hydroxy-6-oxo-2H-pyran-2-yl)-ethyl}-1-naphthalenyl ester, {1S*-{1a,3a,7b,8b(2S*,4S),-8ab}}. The empirical formula of Simvastatin is C₂₅H₃₈O₅ and its molecular weight is 418.57. Pravastatin sodium is designated chemically as 1-Naphthalene-heptanoic acid, 1,2,6,7,8,8a-hexahydro-b,d,6-trihydroxy-2-methyl-8-(2-methyl-1-oxobutoxy)-, monosodium salt, {1S-{1a(bS*,d S*),2a,6a,8b(R*),8aa}}-. Formula C₂₃H₃₅NaO₇, Molecular Weight is 446.52.

Effective treatment with a statin results in lowering of serum cholesterol levels. Such a positive response to statins can be determined by a cholesterol test to determine whether cholesterol levels are lowered as a result of statin administration. Such tests include total cholesterol (TC) and/or low density lipoprotein (LDL) measurements, and are well known and widely used in clinical practice, e.g., methods for determining levels of TC and LDL in blood, especially serum samples. A cholesterol test is often performed to evaluate risks for heart disease. As is known in the art, cholesterol is an important normal body constituent, used in the structure of cell membranes, synthesis of bile acids, and synthesis of steroid hormones. Since cholesterol is water insoluble, most serum cholesterol is carried by lipoproteins (chylomicrons, VLDL, LDL, and HDL). The term “LDL” means LDL-cholesterol and “HDL” means HDL-cholesterol. The term “cholesterol” means total cholesterol (VLDL+LDL+HDL). Excess cholesterol in the blood has been correlated with cardiovascular disease. LDL is sometimes referred to as “bad” cholesterol, because elevated levels of LDL correlate most directly with coronary heart disease. HDL is sometimes referred to as “good” cholesterol since high levels of HDL reduce risk for coronary heart disease.

Preferably, cholesterol is measured after a patient has fasted. In 2001, guidelines from the National Cholesterol Education Panel recommended that all lipid tests be performed fasting and should measure total cholesterol, HDL, LDL and triglycerides. The total cholesterol measurement, as with all lipid measurements, is typically reported in milligrams per deciliter (mg/dL). Typically, the higher the total cholesterol, the more at risk a subject is for heart disease. A value of less than 200 mg/dL is a “desirable” level and places the subject in a group at less risk for heart disease. Levels over 240 mg/dL may put a subject at almost twice the risk of heart disease as compared to someone with a level less than 200 mg/dL. High LDL cholesterol levels may be the best predictor of risk of heart disease.

The statin response-related SNPs and haplotypes of the invention can be used to infer whether a patient's cholesterol levels are more likely to be reduced by statin treatment and, further, whether the patient is likely to exhibit a muscle adverse effect. A patient whose cholesterol levels, e.g. LDL levels or TC levels, are reduced by statin treatment can be referred to as responders. However, for classification of a subject as a Responder, a cutoff cholesterol reduction minimum can be set. For example, a subject can be classified as a Responder if TC or LDL are reduced by at least 1%, or both TC and LDL are reduced by at least 20%.

As used herein, the term “at least one”, when used in reference to a gene, SNP, haplotype, or the like, means 1, 2, 3, 4, 5, 6, 7, 8, 9, 10, etc. Reference to “at least a second” gene, SNP, or the like, for example, a statin response-related gene, means two or more, i.e., 2, 3, 4, 5, 6, 7, 8, 9, 10, etc., statin response-related genes.

The term “haplotypes” as used herein refers to groupings of two or more nucleotide SNPs present in a gene. The term “haplotype alleles” as used herein refers to a non-random combination of nucleotide occurrences of SNPs that make up a haplotype. Haplotype alleles are much like a string of contiguous sequence bases, except the SNPs are not adjacent to one another on a chromosome. For example, SNPs can be included as part of the same haplotype, even if they are thousands of base pairs apart from one another on a genome. Typically, SNPs that make up a haplotype are from the same gene.

Penetrant statin response-related haplotype alleles are haplotype alleles whose association with a statin response is strong enough to be detected using simple genetics approaches. Corresponding haplotypes of penetrant statin response-related haplotype alleles, are referred to herein as “penetrant statin response-related haplotypes.” Similarly, individual nucleotide occurrences of SNPs are referred to herein as “penetrant statin response-related SNP nucleotide occurrences” if the association of the nucleotide occurrence with a statin response is strong enough on its own to be detected using simple genetics approaches, or if the SNP loci for the nucleotide occurrence make up part of a penetrant haplotype. The corresponding SNP loci are referred to herein as “penetrant statin response-related SNPs.” Haplotype alleles of penetrant haplotypes are also referred to herein as “penetrant haplotype alleles” or “penetrant genetic features.” Penetrant haplotypes are also referred to herein as “penetrant genetic feature SNP combinations.” The SNPs disclosed herein, and listed in Tables 1 and 2 below, include both penetrant and latent (see below) statin response-related SNPs, and make up statin response-related penetrant haplotypes.

Examples 1 and 2 provide SNPs, including genomic sequences flanking the SNPs, useful for determining a statin and ACE inhibitor response, respectively. From this information, the SNP loci can be identified within the human genome. It will be recognized that the 5′ and 3′ flanking sequences exemplified herein, provide sufficient information to identify the SNP location within the human genome. However, due to variability in the human genome, in addition to the statin and ACE inhibitor response-related SNPs disclosed herein, as well as sequencing inaccuracy and inaccuracy of information available in public databases, the 5′ and 3′ flanking sequences disclosed herein may not be 100% identical to a database entry, but need not be 100% identical to effectively identify the location of the SNP within a database sequence. However, when the flanking sequences are used to search a database of human genome sequences, it is expected that the highest match in terms of sequence identity will be the entry in the database that corresponds to the location within the human genome that includes the SNP surrounded by those flanking sequences.

Polymorphisms are allelic variants that occur in a population. The polymorphism can be a single nucleotide difference present at a locus, or can be an insertion or deletion of one or a few nucleotides. As such, a single nucleotide polymorphism (SNP) is characterized by the presence in a population of one or two, three or four nucleotides (i.e., adenosine, cytosine, guanosine or thymidine), typically less than all four nucleotides, at a particular locus in a genome such as the human genome. Accordingly, it will be recognized that, while the methods of the invention are exemplified primarily by the detection of SNPs, the disclosed methods or others known in the art similarly can be used to identify other polymorphisms in the exemplified genes or other statin response-related genes.

Simple genetic tests (e.g., Mendelian) can be used for examining alleles include analyzing allele frequencies in populations with different phenotypes for a statin response or ACE inhibitor response being analyzed, to discover those haplotypes that occur more or less frequently in individuals with a certain response, for example, increased muscle adverse effects. In such simple genetics methods SNP nucleotide occurrences are scored and distribution frequencies are analyzed. Haplotypes can be inferred from genotype data corresponding to certain SNPs using the Stephens and Donnelly algorithm (Am. J. Hum. Genet. 68:978-989, 2001). Haplotype phases (i.e., the particular haplotype alleles in an individual) can also be determined using the Stephens and Donnelly algorithm (Am. J. Hum. Genet. 68:978-989, 2001). Software programs are available which perform this algorithm (e.g., The PHASE program, Department of Statistics, University of Oxford).

In one example, called the Haploscope method (U.S. Ser. No. 10/120,804, filed Apr. 11, 2002, the contents of which are incorporated by reference in its entirety) a candidate SNP combination is selected from a plurality of candidate SNP combinations for a gene associated with a genetic trait. Haplotype data associated with this candidate SNP combination are read for a plurality of individuals and grouped into a positive-responding group and a negative-responding group based on whether predetermined trait criteria, such as a statin response, for an individual are met. A statistical analysis (as discussed below) on the grouped haplotype data is performed to obtain a statistical measurement associated with the candidate SNP combination. The acts of selecting, reading, grouping, and performing are repeated as necessary to identify the candidate SNP combination having the optimal statistical measurement. In one approach, all possible SNP combinations are selected and statistically analyzed. In another approach, a directed search based on results of previous statistical analysis of SNP combinations is performed until the optimal statistical measurement is obtained. In addition, the number of SNP combinations selected and analyzed may be reduced based on a simultaneous testing procedure.

As used herein, the term “infer” or “inferring”, when used in reference to a statin response or ACE inhibitor response, means drawing a conclusion about a response using a process of analyzing individually or in combination, nucleotide occurrence(s) of one or more statin or ACE inhibitor response-related SNP(s), respectively, in a nucleic acid sample of the subject, and comparing the individual or combination of nucleotide occurrence(s) of the SNP(s) to known relationships of nucleotide occurrence(s) of the statin response-related SNP(s). In some cases, the inference that can be drawn according to the methods of the present invention are very strong and have great predictive value. In some cases, the inference drawn can be conclusive or determinative of the response analyzed. In other cases, the inference is of a weaker association is weaker and may be associated with a slight or moderate increase in risk of response. Such inferences may be considered indicative rather than conclusive or determinative of the response. As disclosed herein, the nucleotide occurrence(s) can be identified directly by examining nucleic acid molecules, or indirectly by examining a polypeptide encoded by a particular gene, wherein the polymorphism is associated with an amino acid change in the encoded polypeptide.

Methods of performing such a comparison and reaching a conclusion based on that comparison are disclosed in International Publ. No. PCT WO 03/002721 (publ. Jan. 9, 2003) and U.S. patent application Ser. No. 10/188,359, published Nov. 20, 2003 as U.S. Patent Publication No. 20030215819 A1, the entire contents of which are incorporated herein by reference. The inference typically can involve using a complex model that involves using known relationships of known alleles or nucleotide occurrences as classifiers. The comparison can be performed by applying the data regarding the subject's statin response-related haplotype allele(s) to a complex model that makes a blind, quadratic discriminate classification using a variance-covariance matrix. Various classification models are discussed in more detail herein.

To determine whether haplotypes are useful in an inference of a statin response, numerous statistical analyses can be performed. Allele frequencies can be calculated for haplotypes and pair-wise haplotype frequencies estimated using an EM algorithm (Excoffier and Slatkin, Mol Biol Evol. 1995 September; 12(5):921-7). Linkage disequilibrium coefficients can then be calculated. In addition to various parameters such as linkage disequilibrium coefficients, allele and haplotype frequencies, chi-square statistics and other population genetic parameters such as Panmitic indices can be calculated to control for ethnic, ancestral or other systematic variation between the case and control groups.

Markers/haplotypes with value for distinguishing the case matrix from the control, if any, can be presented in mathematical form describing any relationship and accompanied by association (test and effect) statistics. A statistical analysis result which shows an association of a SNP marker or a haplotype with a statin (or ACE inhibitor) response with at least 80%, 85%, 90%, 95%, or 99%. In some embodiments, 95% confidence, or alternatively a probability of insignificance (p value) less than 0.05, can be used to identify haplotypes. These statistical tools may test for significance related to a null hypothesis that an on-test SNP allele or haplotype allele is not significantly different between the groups. If the significance of this difference is low, it suggests the allele is not related to a statin response. The discovery of haplotype alleles can be verified and validated as genetic features for statin response using a nested contingency analysis of haplotype cladograms.

The observance of a number of haplotypes in nature that is far fewer than the number of haplotypes possible is common and appreciated as a general principle among those familiar with the state of the art, and it is commonly accepted that haplotypes offer enhanced statistical power for genetic association studies. This phenomenon is caused by systematic genetic forces such as population bottlenecks, random genetic drift, selection, and the like, which have been at work in the population over time, typically for millions of years, and have created a great deal of genetic “pattern” in the present population. As a result, working in terms of haplotypes offers a geneticist greater statistical power to detect associations, and other genetic phenomena, than working in terms of disjoined genotypes. For larger numbers of polymorphic loci the disparity between the number of observed and expected haplotypes is larger than for smaller numbers of loci.

In diploid organisms such as humans, somatic cells, which are diploid, include two alleles for each haplotype. As such, in some cases, the two alleles of a haplotype are referred to herein as a genotype or as a diploid pair, and the analysis of somatic cells, typically identifies the alleles for each copy of the haplotype. Methods of the present invention can include identifying a diploid pair of haplotype alleles. These alleles can be identical (homozygous) or can be different (heterozygous). The haplotypes of a subject can be symbolized by representing alleles on the top and bottom of a slash (e.g., ATG/CTA or GTT/AGA), where the sequence on the top of the slash represents the combination of polymorphic alleles on the maternal chromosome and the other, the paternal (or vice versa).

The methods of the invention that include identifying a nucleotide occurrence in the sample for at least one statin (or ACE inhibitor) response-related SNP; in preferred embodiments can include grouping the nucleotide occurrences of the statin response-related SNPs into one or more identified haplotype alleles of a statin response-related haplotype. To infer the statin response of the subject, the identified haplotype alleles are then compared to known haplotype alleles of the statin response-related haplotype, wherein the relationship of the known haplotype alleles to the statin response is known.

The methods and compositions of the invention have numerous utilities, the most obvious of which is that they can be used to determine whether to prescribe statins to a patient with elevated serum cholesterol levels or ACE inhibitors to a patient with coronary artery disease. A sample useful for practicing a method of the invention can be any biological sample of a subject that contains nucleic acid molecules, including portions of the gene sequences to be examined, or corresponding encoded polypeptides, depending on the particular method. As such, the sample can be a cell, tissue or organ sample, or can be a sample of a biological fluid such as semen, saliva, blood, and the like. A nucleic acid sample useful for practicing a method of the invention will depend, in part, on whether the SNPs of the haplotype to be identified are in coding regions or in non-coding regions. Thus, where at least one of the SNPs to be identified is in a non-coding region, the nucleic acid sample generally is a deoxyribonucleic acid (DNA) sample, particularly genomic DNA or an amplification product thereof. However, where heteronuclear ribonucleic acid (RNA), which includes unspliced mRNA precursor RNA molecules, is available, a cDNA or amplification product thereof can be used. Where the each of the SNPs of the haplotype is present in a coding region of a gene(s), the nucleic acid sample can be DNA or RNA, or products derived therefrom, for example, amplification products. Furthermore, while the methods of the invention generally are exemplified with respect to a nucleic acid sample, it will be recognized that particular haplotype alleles can be in coding regions of a gene and can result in polypeptides containing different amino acids at the positions corresponding to the SNPs due to non-degenerate codon changes. As such, in another aspect, the methods of the invention can be practiced using a sample containing polypeptides of the subject.

Numerous methods for identifying haplotype alleles in nucleic acid samples (also referred to a surveying the genome) are disclosed herein or otherwise known in the art. As disclosed herein, nucleic acid occurrences for the individual SNPs that make up the haplotype alleles are determined, then, the nucleic acid occurrence data for the individual SNPs is combined to identify the haplotype alleles. The Stephens and Donnelly algorithm (Am. J. Hum. Genet. 68:978-989, 2001, which is incorporated herein by reference) can be applied to the data generated regarding individual nucleotide occurrences in SNP markers of the subject, in order to determine the alleles for each haplotype in the subject's genotype. Other methods that can be used to determine alleles for each haplotype in the subject's genotype, for example Clarks algorithm, and an EM algorithm described by Raymond and Rousset (Raymond et al. 1994. GenePop. Ver 3.0. Institut des Siences de l'Evolution. Universite de Montpellier, France. 1994)

The SNPs disclosed herein include flanking nucleotide sequences, which can serve to aid in the identification of the precise location of the SNPs in the human genome, and serve as target gene segments useful for performing methods of the invention. A target polynucleotide typically includes a SNP locus and a segment of a corresponding gene that flanks the SNP. Primers and probes that selectively hybridize at or near the target polynucleotide sequence, as well as specific binding pair members that can specifically bind at or near the target polynucleotide sequence, can be designed based on the disclosed gene sequences and information provided herein.

Latent statin (or ACE inhibitor) response-related haplotype alleles are haplotype alleles that, in the context of one or more penetrant haplotypes, strengthen the inference of a statin response. Latent statin response-related haplotype alleles are typically alleles whose association with a statin response is not strong enough to be detected with simple genetics approaches. Latent statin response-related SNPs are individual SNPs that make up latent statin response-related haplotypes. It is possible that some of the SNPs which forms statin response-related haplotypes disclosed herein, are latent statin response-related SNPs.

The subject for the methods of the present invention can be a subject of any race. As such, the subject can be of any group of people classified together on the basis of common history, nationality, or geographic distribution. For example, the subject can be of African, Asian, Australia, European, North American, and South American descent. In certain embodiments the subject is Asian, Hispanic, African, or Caucasian. In one embodiment the subject is Caucasian.

As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying a nucleotide occurrence of a SNP. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments, it can be useful to detectably label a polynucleotide or oligonucleotide. Detectable labeling of a polynucleotide or oligonucleotide is well known in the art. Particular non-limiting examples of detectable labels include chemiluminescent labels, radiolabels, enzymes, haptens, or even unique oligonucleotide sequences.

A method of the identifying a SNP also can be performed using a specific binding pair member. As used herein, the term “specific binding pair member” refers to a molecule that specifically binds or selectively hybridizes to another member of a specific binding pair. Specific binding pair member include, for example, probes, primers, polynucleotides, antibodies, etc. For example, a specific binding pair member includes a primer or a probe that selectively hybridizes to a target polynucleotide that includes a SNP loci, or that hybridizes to an amplification product generated using the target polynucleotide as a template.

As used herein, the term “specific interaction,” or “specifically binds” or the like means that two molecules form a complex that is relatively stable under physiologic conditions. The term is used herein in reference to various interactions, including, for example, the interaction of an antibody that binds a polynucleotide that includes a SNP site; or the interaction of an antibody that binds a polypeptide that includes an amino acid that is encoded by a codon that includes a SNP site. According to methods of the invention, an antibody can selectively bind to a polypeptide that includes a particular amino acid encoded by a codon that includes a SNP site. Alternatively, an antibody may preferentially bind a particular modified nucleotide that is incorporated into a SNP site for only certain nucleotide occurrences at the SNP site, for example using a primer extension assay.

A specific interaction can be characterized by a dissociation constant of at least about 1×10⁻⁶ M, generally at least about 1×10⁻⁷M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

Numerous methods are known in the art for determining the nucleotide occurrence for a particular SNP in a sample. Such methods can utilize one or more oligonucleotide probes or primers, including, for example, an amplification primer pair, that selectively hybridize to a target polynucleotide, which contains one or more statin response-related SNP positions. Oligonucleotide probes useful in practicing a method of the invention can include, for example, an oligonucleotide that is complementary to and spans a portion of the target polynucleotide, including the position of the SNP, wherein the presence of a specific nucleotide at the position (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe.

An oligonucleotide ligation assay also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method” (Sanger, F., et al., J. Molec. Biol. 94:441 (1975); Prober et al. Science 238:336-340 (1987)) and the “chemical degradation method,” “also known as the “Maxam-Gilbert method” (Maxam, A. M., et al., Proc. Natl. Acad. Sci. (U.S.A.) 74:560 (1977)), both references herein incorporated by reference) to determine the nucleotide occurrence at the SNP loci.

Methods of the invention can identify nucleotide occurrences at SNPs using a “microsequencing” method. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP loci are discussed in Boyce-Jacino, et al., U.S. Pat. No. 6,294,336, incorporated herein by reference, and summarized herein.

Microsequencing methods include the Genetic Bit Analysis method disclosed by Goelet, P. et al. (WO 92/15712, herein incorporated by reference). Additional, primer-guided, nucleotide incorporation procedures for assaying polymorphic sites in DNA have also been described (Komher et al, Nucl. Acids. Res. 17:7779-7784 (1989); Sokolov, Nucl. Acids Res. 18:3671 (1990); Syvanen, et al., Genomics 8:684-692 (1990); Kuppuswamy et al., Proc. Natl. Acad. Sci. (U.S.A.) 88:1143-1147 (1991); Prezant et al, Hum. Mutat. 1:159-164 (1992); Ugozzoli et al., GATA 9:107-112 (1992); Nyren et al., Anal. Biochem. 208:171-175 (1993); and Wallace, WO89/10414). These methods differ from Genetic Bit™ methods. Analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. Amer. J. Hum. Genet. 52:46-59 (1993)).

Alternative microsequencing methods have been provided by Mundy (U.S. Pat. No. 4,656,127) and Cohen, D. et al (French Patent 2,650,840; PCT Appl. No. WO91/02087) which discusses a solution-based method for determining the identity of the nucleotide of a polymorphic site. As in the Mundy method of U.S. Pat. No. 4,656,127, a primer is employed that is complementary to allelic sequences immediately 3′-to a polymorphic site.

In response to the difficulties encountered in employing gel electrophoresis to analyze sequences, alternative methods for microsequencing have been developed. Macevicz (U.S. Pat. No. 5,002,867), for example, describes a method for determining nucleic acid sequence via hybridization with multiple mixtures of oligonucleotide probes. In accordance with such method, the sequence of a target polynucleotide is determined by permitting the target to sequentially hybridize with sets of probes having an invariant nucleotide at one position, and a variant nucleotides at other positions. The Macevicz method determines the nucleotide sequence of the target by hybridizing the target with a set of probes, and then determining the number of sites that at least one member of the set is capable of hybridizing to the target (i.e., the number of “matches”). This procedure is repeated until each member of a sets of probes has been tested.

Boyce-Jacino, et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

In one particular commercial example of a method that can be used to identify a nucleotide occurrence of one or more SNPs, the nucleotide occurrences of statin response-related SNPs in a sample can be determined using the SNP-IT™ method (Orchid BioSciences, Inc., Princeton, N.J.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument (Orchid BioSciences, Inc., Princeton, N.J.).

In another embodiment, a method of the present invention can be performed by amplifying a polynucleotide region that includes a statin response-related SNP, capturing the amplified product in an allele specific manner in individual wells of a microtiter plate, detecting the captured target allele. Phase known data can be generated by inputting phase unknown raw data from the SNPstream™ instrument into the Stephens and Donnelly's PHASE program.

Accordingly, using the methods described above, the statin response-related haplotype allele or the nucleotide occurrence of the statin response-related SNP can be identified using an amplification reaction, a primer extension reaction, or an immunoassay. The statin response-related haplotype allele or the statin response-related SNP can also be identified by contacting polynucleotides in the sample or polynucleotides derived from the sample, with a specific binding pair member that selectively hybridizes to a polynucleotide region comprising the statin response-related SNP, under conditions wherein the binding pair member specifically binds at or near the statin response-related SNP. The specific binding pair member can be an antibody or a polynucleotide.

Antibodies that are used in the methods of the invention include antibodies that specifically bind polynucleotides that encompass a statin response-related or race-related haplotype. In addition, antibodies of the invention bind polypeptides that include an amino acid encoded by a codon that includes a SNP. These antibodies bind to a polypeptide that includes an amino acid that is encoded in part by the SNP. The antibodies specifically bind a polypeptide that includes a first amino acid encoded by a codon that includes the SNP loci, but do not bind, or bind more weakly to a polypeptide that includes a second amino acid encoded by a codon that includes a different nucleotide occurrence at the SNP.

Antibodies are well-known in the art and discussed, for example, in U.S. Pat. No. 6,391,589. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Antibodies of the invention include antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. The antibodies of the invention may be monospecific, bispecific, trispecific or of greater multispecificity.

The antibodies of the invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacillus Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example; in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Where the particular nucleotide occurrence of a SNP, or nucleotide occurrences of a statin response-related haplotype, is such that the nucleotide occurrence results in an amino acid change in an encoded polypeptide, the nucleotide occurrence can be identified indirectly by detecting the particular amino acid in the polypeptide. The method for determining the amino acid will depend, for example, on the structure of the polypeptide or on the position of the amino acid in the polypeptide.

Where the polypeptide contains only a single occurrence of an amino acid encoded by the particular SNP, the polypeptide can be examined for the presence or absence of the amino acid. For example, where the amino acid is at or near the amino terminus or the carboxy terminus of the polypeptide, simple sequencing of the terminal amino acids can be performed. Alternatively, the polypeptide can be treated with one or more enzymes and a peptide fragment containing the amino acid position of interest can be examined, for example, by sequencing the peptide, or by detecting a particular migration of the peptide following electrophoresis. Where the particular amino acid comprises an epitope of the polypeptide, the specific binding, or absence thereof, of an antibody specific for the epitope can be detected. Other methods for detecting a particular amino acid in a polypeptide or peptide fragment thereof are well known and can be selected based, for example, on convenience or availability of equipment such as a mass spectrometer, capillary electrophoresis system, magnetic resonance imaging equipment, and the like.

The method can include identifying a nucleotide occurrence of each of at least two (e.g., 2, 3, 4, 5, 6, or more) statin (or ACE inhibitor) response-related SNPs, which can, but need not comprise one or more haplotype alleles, and can, but need not be in one gene. The nucleotide occurrence of the at least one statin response-related SNP can be a minor nucleotide occurrence, i.e., a nucleotide present in a relatively smaller percent of a population including the subject, or can be a major nucleotide occurrence. Minor nucleotide occurrences are generally associated with a higher probability of an adverse response. Where a haplotype allele is determined, the haplotype allele can be a major haplotype allele, or a minor haplotype allele.

A variety of commonly prescribed medications cause what are commonly considered to be “benign” side effects. Though surrogate markers of adverse response for many FDA approved drugs usually self resolve and are thought to be of little consequence for long term health, there may be more sinister relationships between aberrant surrogate marker test results and long term health than originally thought (Baker et al., 2001; Amacher et al., 2001).

About 3% of patients who take Statins develop symptoms of hepatocellular (liver) injury. A greater percent of patients exhibit myalgia or muscle pain. Prolonged use in those individuals that exhibit adverse response to Statins can, and does lead to permanent disease. For example, clinical trials showed that about 1% of Baycol patients (similar to other Statins), experienced muscle discomfort and/or creatine kinase elevations in response to treatment. Nonetheless, it took several years of post-trial drug use to illustrate that the relatively high frequency of minor complaints and surrogate marker abnormalities were part of a continuum of clinical pathology that extends, in its extreme, to myonecrosis and even death.

The incidence of statin induced hepatocellular stress may likewise portend a serious health risks in the Statin patient population (Rienus, 2000). Though Statin induced hepatic stress usually resolves on its own, in some patients it worsens to hepatic injury indicated by decreases in liver weight, jaundice, hepatitis or even death.

An “adverse statin response” is any negative response to statins, particularly a muscle adverse response such as a myopathy, rhabdomyolysis, and the like. Methods for identifying a muscle adverse effect are well known and include, for example, measuring creatine kinase, wherein increased levels above normal are indicative of adverse response to statins. About 20% of patients who take statins complain of muscle ache, and elevated creatine kinase levels are indicative of myalgia (muscle injury).

Hepatic stress is another example of a negative response to a statin, possibly accompanied by liver damage. A negative hepatocellular response according to the present invention is inferred by identifying nucleotide occurrences, and optionally haplotypes, of the CYP2D6 gene. Approximately 0.7% of patients taking Atorvastatin exhibit persistent and dose-dependent indications of hepatic stress, the most commonly observed being an elevation in serum transaminase (SGOT, ALTGPT) levels. These and other indications of hepatic stress are indicators of an adverse statin response according to this aspect of the invention. Because drug induced hepatocellular damage is preceded by elevations in liver function tests, physicians routinely perform these tests prior to, at 12 weeks and periodically following the initiation of (or increase in dosage of) Statins and discontinue treatment if the elevations persist. Though clinical trials have shown that only a minor proportion of patients exhibit what are considered “dangerous” SGOT and GPT elevations (the classification of which is entirely arbitrary), it is common knowledge that a significantly higher proportion of patients (up to 30%, unpublished observations) exhibit more modest, but significant elevations greater than 20% of baseline. Additionally, For the average individual, an increase in the SGOT level to 37 or higher, or an increase in the GPT level above 56 signifies an adverse hepatocellular response. However, these thresholds are relevant to the average human, without regard to their race, sex or age.

Because the incidence of aberrant surrogate marker levels in response to drugs like statins is not small, various laboratories have investigated whether drug pretreatment regimens diminish the severity of adverse hepatocellular injury caused by some drugs by decreasing oxidative stress and lipoperoxidation. The results of these studies indicate that direct measures of hepatocellular health, such as hepatocellular regeneration or DNA fragmentation, are often left unaffected by these pretreatments (Ferrali et al., 1997). The results further suggest that a potential drug-based resolution of statin induced hepatocellular stress may not always proceed without sequelae, and that genetic tests to match patients with Statins may be more effective modality of prophylaxis.

Before the present invention, it was not possible to predict which patients are at risk of having a muscle adverse effect to statin treatment. For virtually all cytochrome P450s, little is known about interactions of alleles between genes (epistasis) or to what extent pharmacogenomic concepts can be integrated with haploid sets of SNPs and environmental components to explain variance in drug response. The expansion of the new field of pharmacogenomics promises to help us more systematically define the role of drug metabolizer variants in drug response. It is hoped that systematic candidate gene approaches (involving multiple genes per project), multiple markers within each gene, and intensely annotated patient databanks can be economically screened to find new and/or complimentary pharmacogenomics marker sets that explain a greater percent of drug reaction trait variability in the population than previously found. Polymorphisms in the CYP2D6 gene, for example, have been previously discovered by others to be deterministic for undesirable reaction to a variety of commonly prescribed medications Kalow, Pergamon Press, Pharmacogenetics of Drug Metabolism). Catastrophic, Mendelian mutations in this gene have also been associated with various adverse events associated with the use of various drugs. As disclosed herein, natural variation in this gene also is related to variable efficacy of the statins, including commonly observed muscle adverse effects.

The present invention provides a method for inferring a statin response of a human subject from a nucleic acid sample of the subject, wherein the method includes identifying, in the nucleic acid sample, a nucleotide occurrence of at least one statin response-related single nucleotide polymorphism (SNP) in one of the genes listed in Example 1, whereby the nucleotide occurrence is associated with muscle adverse effect in response to administration of a statin. The present invention also provides a method for inferring an ACE inhibitor response of a human subject from a nucleic acid sample of the subject, wherein the method includes identifying, in the nucleic acid sample, a nucleotide occurrence of at least one ACE inhibitor response-related SNP in one of the genes listed in Example 2, whereby the nucleotide occurrence is associated with a dry cough adverse effect in response to administration of the ACE inhibitor.

The present invention also related to an isolated human cell or an isolated plurality of cells, which contain a minor nucleotide occurrence of a statin (or ACE inhibitor) response-related SNP or a minor haplotype allele. The cells are useful for drug design, for example of new, more effective statins (or ACE inhibitors) that exhibit fewer side effects. For example, the cells can be used to screen test agents, such as new statins, for efficacy and propensity to elicit an adverse response. Bioassays of test agents using the isolated cells can for example, screen the agent for an effect on activity, such as enzymatic activity, of a CYP2D6 protein. Furthermore, efficacy of an on-test agent can be determined by measuring cholesterol uptake and/or metabolism in the isolated cells. In certain preferred embodiments, the cells are cultured myocytes. Methods are known in the art for testing agents such as statins, on isolated cells, including hepatocytes, for inhibition of CYP2D6 activity (See e.g., Cohen et. al. Biopharm. Drug Dispos. 21:353 (2002)). Isolated cells of the present invention can also be cultured and used to make microsomal preparations for assaying effects of agents such as statins on the activity of CYP2D6.

Enzyme activity for CYP2D6 after exposure to a statin, such as Atorvastatin, can be analyzed in isolated cells of the present invention, which have at least one minor nucleotide occurrence in at least one statin response-related SNP, and compared to enzyme activity after exposure to the statin of isolated cells which have a major (i.e. wild type) nucleotide occurrence in the corresponding statin response-related SNP, to identify isolated cells which exhibit a different enzymatic activity after exposure to the statin, than cells with a major nucleotide occurrence. This step can be helpful because certain subjects with a minor nucleotide occurrence in a statin response-related SNP can exhibit an efficacious statin response and/or no adverse reactions.

A method of identifying an agent can be performed, for example, by contacting an isolated cell of the present invention with at least a test agent to be examined as a potential agent for treating elevated serum cholesterol, and detecting an effect on the activity of CYP2D6. In certain embodiments, an effect on the activity of CYP2D56 can be determined by comparing the effect on isolated cells of the present invention which include a minor nucleotide occurrence of a statin response-related SNP, to cells which include a major occurrence at the statin response-related SNP.

The term “test agent” is used herein to mean any agent that is being examined for the ability to affect the activity of a gene product. The method generally is used as a screening assay to identify previously unknown molecules that can act as a therapeutic agent for treating elevated cholesterol levels. A test agent can be any type of molecule, including, for example, a peptide, a peptidomimetic, a polynucleotide, or a small organic molecule, that one wishes to examine for the ability to act as a therapeutic agent, which is a agent that provides a therapeutic advantage to a subject receiving it. It will be recognized that a method of the invention is readily adaptable to a high throughput format and, therefore, the method is convenient for screening a plurality of test agents either serially or in parallel. The plurality of test agents can be, for example, a library of test agents produced by a combinatorial method library of test agents. Methods for preparing a combinatorial library of molecules that can be tested for therapeutic activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., supra, 1996; Tuerk and Gold, supra, 1990; Gold et al., supra, 1995; each of which is incorporated herein by reference); an oligosaccharide library (York et al., Carb. Res., 285:99-128, 1996; Liang et al., Science, 274:1520-1522, 1996; Ding et al., Adv. Expt. Med. Biol., 376:261-269, 1995; each of which is incorporated herein by reference); a lipoprotein library (de Kruif et al., FEBS Lett., 399:232-236, 1996, which is incorporated herein by reference); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, BioTechnology, 13:351-360, 1995; each of which is incorporated herein by reference). Accordingly, the present invention also provides a therapeutic agent identified by such a method, for example, a cancer therapeutic agent.

Assays that utilize these cells to screen test agents are typically performed on isolated cells of the present invention in tissue culture. The isolated cells can be cells from a cell line, passaged primary cells, or primary cells, for example. An isolated cell according to the present invention can be, for example, a myocyte, or a myocyte cell line. The present invention also relates to a plurality of isolated human cells, which includes at least two (e.g., 2, 3, 4, 5, 6, 7, 8, or more) populations of isolated cells, wherein the isolated cells of one population contain at least one nucleotide occurrence statin (or ACE inhibitor) response related SNP or at least one statin response related haplotype allele that is different from the isolated cells of at least one other population of cells of the plurality.

In another embodiment the present invention provides a vector containing one or more of the isolated polynucleotides disclosed herein. Many vectors are known in the art, including expression vectors. In one aspect, the vectors of the present invention include an isolated polynucleotide of the present invention that encodes a polypeptide, operatively linked to an expression control sequence such as a promoter sequence on the vector. Sambrook (1989) for example, provides examples of vectors and methods for manipulating vectors, which are well known in the art.

In another embodiment, the present invention provides an isolated cell containing one or more of the isolated polynucleotides disclosed herein, or one or more of the vectors disclosed in the preceding sentence. As such, the cell is a recombinant cell.

The present invention also relates to an isolated primer pair, which can be useful for amplifying a nucleotide sequence comprising a SNP in a polynucleotide, wherein a forward primer of the primer pair selectively binds the polynucleotide upstream of the SNP position on one strand and a reverse primer selectively binds the polynucleotide upstream of the SNP position on a complementary strand, wherein the polynucleotide includes a nucleotide occurrence corresponding to a SNP position of the nucleotide sequences set forth in Examples 1 and 2. The present invention also relates to an isolated primer pair, which can be useful for amplifying a nucleotide sequence comprising a SNP in a polynucleotide, wherein a forward primer of the primer pair selectively binds the polynucleotide upstream of the SNP position on one strand and a reverse primer selectively binds the polynucleotide upstream of the SNP position on a complementary strand, wherein the polynucleotide includes a nucleotide occurrence as disclosed herein. The isolated primer pair can include a 3′ nucleotide that is complementary to one nucleotide occurrence of the statin (or ACE inhibitor) response-related SNP. Accordingly, the primer can be used to selectively prime an extension reaction to polynucleotides wherein the nucleotide occurrence of the SNP is complementary to the 3′ nucleotide of the primer pair, but not polynucleotides with other nucleotide occurrences at a position corresponding to the SNP. Randomly selected primers about 20 nucleotides in length, for example, from the five prime and three-prime sequence included in the sequence listing, can be used as primers according to the present invention provided that the A/T:G/C ratios are similar within each primer.

In another embodiment the present invention provides an isolated probe for determining a nucleotide occurrence of a single nucleotide polymorphism (SNP) in a polynucleotide, wherein the probe selectively binds to a polynucleotide as set forth in Examples 1 and 2, including an alternative nucleotide of the SNP position. In another embodiment, the present invention provides an isolated primer for extending a polynucleotide. The isolated polynucleotide includes a single nucleotide polymorphism (SNP), wherein the primer selectively binds the polynucleotide upstream of the SNP position as disclosed in Examples 1 and 2.

The present invention further relates to an isolated specific binding pair member, which can be useful for determining a nucleotide occurrence of a SNP in a polynucleotide, wherein the specific binding pair member specifically binds to an alternative nucleotide of a SNP position as disclosed in Examples 1 and 2. For methods wherein the specific binding pair member is a substrate for a primer extension reaction, the specific binding pair member is a primer that binds to a polynucleotide at a sequence comprising the SNP as the terminal nucleotide. As discussed above, methods such as SNP-IT (Orchid BioSciences), utilize primer extension reactions using a primer whose terminal nucleotide binds selectively to certain nucleotide occurrence(s) at a SNP loci, to identify a nucleotide occurrence at the SNP loci.

The polynucleotides of the present invention have many uses. For example, the polynucleotides can be used in recombinant DNA technologies to produce recombinant polypeptides that can be used, for example, to determine whether a statin binds or effects activity of the polypeptide. The present invention also provides isolated polypeptides that are produced using the isolated polynucleotides of the present invention. In another aspect, the invention provides a method for identifying genes, including statin response genes, SNPs, SNP alleles, haplotypes, and haplotype alleles that are statistically associated with a statin response. This aspect of the invention provides commercially valuable research tools, for example. The approach can be performed generally as follows:

1) Select genes from the human genome database that are likely to be involved in a statin response;

2) Identify the common genetic variations in the selected genes by designing primers to flank each promoter, exon and 3′ UTR for each of the genes; amplifying and sequencing the DNA corresponding to each of these regions in enough donors to provide a statistically significant sample; and utilize an algorithm to compare the sequences to one another in order to identify the positions within each region of each gene that are variable in the population, to produce a gene map for each of the relevant genes;

3) Use the gene maps to design and execute large-scale genotyping experiments, whereby a significant number of individuals, typically at least one hundred, more preferably at least two hundred individuals, of known statin response are scored for the polymorphisms; and

4) Use the results obtained in step 3) to identify genes, polymorphisms, and sets of polymorphisms, including haplotypes, that are quantitatively and statistically associated with a statin response.

The Examples included herein illustrate general approaches for discovering statin response-related SNPs and SNP alleles as provided above.

The invention also relates to kits, which can be used, for example, to perform a method of the invention. Thus, in one embodiment, the invention provides a kit for identifying SNPs and/or haplotype alleles of statin response-related SNPs and/or ACE inhibitor response-related SNPs. Such a kit can contain, for example, an oligonucleotide probe, primer, or primer pair, or combinations thereof, of the invention, such oligonucleotides being useful, for example, to identify a SNP or haplotype allele as disclosed herein; or can contain one or more polynucleotides corresponding to a portion of a gene, including the SNP position, as set forth in Examples 1 and 2, such a polynucleotide being useful, for example, as a standard (control) that can be examined in parallel with a test sample. In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease), which can be useful for performing an oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin or an antibody.

In one embodiment, a kit of the invention includes one or more primer pairs of the invention, such a kit being useful for performing an amplification reaction such as a polymerase chain reaction (PCR). Such a kit also can contain, for example, one or reagents for amplifying a polynucleotide using a primer pair of the kit. The primer pair(s) can be selected, for example, such that they can be used to determine the nucleotide occurrence of a statin response-related SNP, wherein a forward primer of a primer pair selectively hybridizes to a sequence of the target polynucleotide upstream of the SNP position on one strand, and the reverse primer of the primer pair selectively hybridizes to a sequence of the target polynucleotide upstream of the SNP position on a complementary strand. When used together in an amplification reaction an amplification product is formed that includes the SNP loci.

In addition to primer pairs, in this embodiment the kit can further include a probe that selectively hybridizes to the amplification product of one of the nucleotide occurrences of a SNP, but not the other nucleotide occurrence. Also in this embodiment, the kit can include a third primer which can be used for a primer extension reaction across the SNP loci using the amplification product as a template. In this embodiment the third primer preferably binds to the SNP loci such that the nucleotide at the 3′ terminus of the primer is complementary to one of the nucleotide occurrences at the SNP loci. The primer can then be used in a primer extension reaction to synthesize a polynucleotide using the amplification product as a template, preferably only where the nucleotide occurrence is complementary to the 3′ nucleotide of the primer. The kit can further include the components of the primer extension reaction.

In another embodiment, a kit of the invention provides a plurality of oligonucleotides of the invention, including one or more oligonucleotide probes or one or more primers, including forward and/or reverse primers, or a combination of such probes and primers or primer pairs. Such a kit provides a convenient source for selecting probe(s) and/or primer(s) useful for identifying one or more SNPs or haplotype alleles as desired. Such a kit also can contain probes and/or primers that conveniently allow a method of the invention to be performed in a multiplex format. A kit can also include instructions for using the probes or primers to identify a statin or ACE inhibitor response-related SNP and/or haplotype allele.

The inference drawn according to the methods of the invention can utilize a complex classifier function. However, as illustrated in Example 1 (see, also, PCT International Publ. No. WO 03/002721, published Jan. 9, 2003, and U.S. patent application Ser. No. 10/188,359, published Nov. 20, 2003 as U.S. Patent Publication No. 20030215819 A1, the entire contents of which are incorporated herein by reference), simple classifier systems can be used with the statin or ACE inhibitor response-related SNPs and haplotypes to infer statin or ACE inhibitor response, respectively. However, the methods of the invention, which draw an inference regarding a statin or ACE inhibitor response of a subject can use a complex classification function. A classification function applies nucleotide occurrence information identified for a SNP or set of SNPs such as one or preferably a combination of haplotype alleles, to a set of rules to draw an inference regarding a statin response (see, e.g., U.S. Ser. No. 10/156,995, filed May 28, 2002, which is incorporated herein by reference and provides examples of complex classifier methods).

### EXAMPLE 1

### Identification of SNPs for Inferring Muscle Adverse Effects Due to Statin Treatment

This example demonstrates that single nucleotide polymorphisms (SNPs) can be identified that allow an inference to be drawn as to whether an individual in more or less likely to have a muscle adverse effect due to treatment with Lipitor® or Zocor®.

The alleles of 205 xenobiotic metabolism SNPs were initially screened in order to identify those with a statistical association with LIPITOR or ZOCOR response (or Enalapril or Lisinopril; see Example 2). Screening was performed as described in PCT International Publ. No. WO 03/002721 (published Jan. 9, 2003) and U.S. patent application Ser. No. 10/188,359 (published Nov. 20, 2003 as U.S. Patent Publication No. 20030215819 A1; the entire contents of which are incorporated herein by reference which is incorporated herein by reference), and as described below.

Not all individuals who develop an adverse response to statins harbor the same SNP types at the same loci. This result is not unexpected, as most traits in the human population are the function of complex gene-gene and gene-environment interactions. If a gene product is involved in the metabolism of a given drug, several different polymorphisms in this gene may impair the function of the gene product and thus, the metabolism of the drug. One person may harbor one particular debilitating polymorphism, and another person may harbor another. Thus, on a population level, it is expected that several polymorphisms in the gene can be associated with adverse events associated with use of the drug.

Briefly, the strength of association is measured with a delta value (Shriver et al., Am. J. Genet., (2002), Shriver et al., Am. J. Genet., 60:1558 (1997), each of which is incorporated herein by reference), which is inversely related to a chi-square statistic (the higher the value, the stronger the association). The delta value measures the difference in allele ratios between one group (in this case, responders) and another (in this case, non-responders). Generally, SNPs with delta values greater than 0.15 are selected, though because the delta value is not very sensitive for sample size, those with delta values above 0.15 that have fewer than 20 counts for the minor allele in the overall sample (responders and non responders combined) generally are discarded.

Gene markers were selected based on evidence from the scientific and medical literature, and from other sources of information, that implicate them in responsiveness to statins. The Physicians Desk Reference, Online Mendelian Inheritance database (e.g. NCBI) and PubMed/Medline are Examples of sources used for this information. Polymorphisms were identified using raw human genomic data present in public data resources (NCBI database) using data mining tools. The NCBI SNP database, the Human Genome Unique Gene database (Unigene from NCBI) and a DNA sequence database generated for this and similar studies, were used as sources for this raw sequence data. Sequence files for the genes were downloaded from proprietary and public databases and saved as a text file in FASTA format and analyzed using a multiple sequence alignment tool. The text file that was obtained from this analysis served as the input for SNP/HAPLOTYPE automated pipeline discovery software system (as described in U.S. patent application Ser. No. 09/964,059, filed Sep. 26, 2001, which is incorporated herein by reference in its entirety). This method finds candidate SNPs among the input sequences and documents haplotypes for the sequences with respect to the identified SNPs. The method uses a variety of quality control metrics when selecting candidate SNPs including the user specified stringency variables, the PHRED quality control scores and others.

The public genome database was constructed from a relatively small collection of donors. In order to discover new SNPs that may be under-represented or biased against in the public human SNP and Unigene databases, the CYP2D6 gene was completely sequenced in a larger pool (n=500) of persons (the DNA specimens were obtained from the Coriell Institute, Camden, N.J.). Specimens from this combined pool were used as a template for amplification using a combination of Pfu turbo thermostable DNA polymerase and Taq polymerase. Amplification was performed in the presence of 1.5 nM MgCl₂, 5m M KCl, 1 mM Tris, pH 9.0, and 0.1% Triton X-100 nonionic detergent. Amplification products were cloned into a T-vector using the Clontech (Palo Alto, Calif.) PCR Cloning Kit, transformed into Calcium Chloride Competent cells (Stratagene; La Jolla Calif.), plated on LB-Ampicillin plates and grown overnight.

Clones were selected from each plate, isolated by a miniprep procedure using the Promega Wizard or Qiagen Plasmid Purification Kit, and sequenced using standard PE Applied Biosystems Big Dye Terminator Sequencing Chemistry. Sequences were deposited into an internet based relational database system, trimmed of vector sequence and quality trimmed. Genotypes were surveyed within the specimen cohorts by sequencing using Klenow fragment-based single base primer extension and an automated Orchid Biosciences SNPstream instrument, based on Dye linked immunochemical recognition of base incorporated during extension. Reactions were processed in 384-well format and the data stored in a temporary database application until transferred to a UNIX based SQL database.

The data corresponded to SNPs that are informative for distinguishing common genetic haplotypes that were identified from public and private databases. Using algorithms, the data was used to infer haplotypes from empirically determined SNP sequences. Allele frequencies were calculated and pair-wise haplotype frequencies estimated using an EM algorithm (Excoffier & Slatkin, Mol Biol Evol. 12:921-7 (1995)). Linkage disequilibrium (LD) coefficients were then calculated. The analytical approach was based on the case-control study design. Genotype/biographical data matrices for each group were examined using a pattern detection algorithm. The purpose of these algorithms was to fit quantitative (or Mendelian) genetic data with continuous trait distributions (or discrete trait distributions). In addition to various parameters such as LD coefficients, allele and haplotype frequencies (within ethnic, control and case groups), chi-square statistics and other population genetic parameters (such as Panmitic indices) were calculated to control for systematic variation between the case and control groups. Markers/haplotypes with value for distinguishing the case matrix from the control, if any, were presented in mathematical form describing their relationship(s) and accompanied by association (test and effect) statistics.

### Lipitor® Study

A set of 205 xenobiotic metabolism SNPs, including SNPs in the AHR (SEQ ID NO: 125), CYP1A2 (SEQ ID NO: 127), CYP2B6 (SEQ ID NO: 130), CYP2C8 (SEQ ID NO: 131), CYP2C9 (SEQ ID NO: 132), CYP2D6 (SEQ ID NO: 134), CYP3A4 (SEQ ID NO: 136), CYP3A5 (SEQ ID NO: 137), GSTM1 (SEQ ID NO: 141), GSTM3 (SEQ ID NO: 141), HMGCR (SEQ ID NO: 143), MVK (SEQ ID NO: 144), PON1 (SEQ ID NO: 145), PON3 (SEQ ID NO: 146), CYP1A1 (SEQ ID NO: 126), CYP2C19 (SEQ ID NO: 133), CYP3A4 (SEQ ID NO: 136), CYP3A7 (SEQ ID NO: 138), CYP2E1 (SEQ ID NO: 135), CYP1B1 (SEQ ID NO: 128), CYP2A6 (SEQ ID NO: 129), CYP4B1 (SEQ ID NO: 139), and GSTP1 (SEQ ID NO: 142), were screened as described above and as described in PCT International Publ. No. WO 03/002721 (published Jan. 9, 2003) and U.S. patent application Ser. No. 10/188,359 (published Nov. 20, 2003 as U.S. Patent Publication No. 20030215819 A1) the entire contents of which are incorporated herein by reference. The top SNPs, ranked by delta value are shown in Table 1 (LIPITORDELTA). As shown in Table 1, of the 22 genes screened, four genes —CYP2D6, CYP2C8, HMGCR and GSTP1—showed multiple hits, which indicate that the results obtained are not random with respect to gene level of analysis.

TABLE 1
LIPITORDELTA

| Sample size | Delta | Marker | |
|---|---|---|---|
| 84 | 0.194529 | 179 | CYP2D6_RS1058174_TC |
| 86 | 0.156621 | 391 | CYP2C8_RS1934952_GA |
| 52 | 0.153846 | 2689 | HMGCR_41344_712043_GA |
| 88 | 0.135455 | 2760 | CYP2C8_2185571_TC |
| 88 | 0.134545 | 381 | CYP2C9RS2253635_GA |
| 88 | 0.132727 | 362 | CYP2C8_2071426_GA |
| 88 | 0.128636 | 58 | GSTP1E2E4-5_419_CA |
| 88 | 0.128636 | 424 | GSTP1E2E4-3_466_GT |
| 76 | 0.117325 | 216 | CYP2C19E7-5_169_TC |
| 88 | 0.115 | 374 | CYP2C8_RS1934953_GA |
| 84 | 0.109048 | 172 | CYP2D6_RS2267446_TC |
| 88 | 0.107727 | 369 | CYP2C8_RS1891071_GA |
| 86 | 0.103953 | 282 | CYP2C8E8_296_GA |
| 88 | 0.103636 | 225 | CYP2C8_RS1891073_TC |
| 86 | 0.101163 | 2680 | HMGCRE711_635_756249_GA |
| 84 | 0.100476 | 98 | CYP1A2E7_405_GC |
| 88 | 0.100379 | 188 | CYP2B6RS1042389_TC |
| 86 | 0.097868 | 2657 | HMGCRE5E6_283_809125_TC |

A similar screen of 10,000 SNPs on a DNA chip (Affymetrix) revealed several other markers associated with adverse muscle reactions to Lipitor®. These results are shown in Table 2 (AFFYLIPMUSC).

TABLE 2
AFFYLIPMUSC

| Sample size | Delta | Marker | P value | Marker |
|---|---|---|---|---|
| 20 | 0.170833 | 2460 | 0.005 | 2406 |
| 86 | 0.148552 | 2468 | 0.005 | 2432 |
| 88 | 0.147727 | 2406 | 0.011 | 2443 |
| 88 | 0.147273 | 2405 | 0.018 | 2405 |
| 88 | 0.145909 | 2409 | 0.018 | 2465 |
| 84 | 0.132381 | 2432 | 0.021 | 2409 |
| 80 | 0.130585 | 2419 | 0.022 | 2468 |
| 86 | 0.129302 | 2450 | 0.034 | 2433 |
| 88 | 0.128636 | 2443 | 0.034 | 2450 |
| 88 | 0.127273 | 2433 | 0.037 | 2391 |
| 84 | 0.111905 | 2445 | 0.04 | 2447 |
| 80 | 0.1 | 2425 | 0.041 | 2419 |
| 88 | 0.099545 | 2407 | 0.045 | 2392 |
| 86 | 0.093256 | 2437 | 0.047 | 2426 |
| 86 | 0.090465 | 2418 | 0.05 | 2394 |
| 84 | 0.083333 | 2394 | 0.05 | 2445 |
| 84 | 0.077143 | 2472 | 0.052 | 2407 |
| 86 | 0.076977 | 2435 | 0.059 | 2418 |
| 88 | 0.076364 | 2463 | 0.069 | 2460 |
| 82 | 0.073668 | 2426 | 0.071 | 2455 |
| 80 | 0.073469 | 2457 | 0.076 | 2463 |
| 84 | 0.070952 | 2404 | 0.082 | 2425 |
| | | | 0.102 | 2437 |

The SNPs identified above were used to make classifications of patients receiving Lipitor®. Genotypes were determined from a sample (e.g. blood) from patients suffering muscle adverse effects (case) and from patients not suffering muscle adverse effects (controls). Genotypes were selected for which the prevalence between cases and controls was more extreme than 63%:37%, or 37%:63% (giving a risk of non-response in terms of classification theory of about 3). As shown in Table 3, the results demonstrated that a Lipitor® patient could be classified into the non-adverse response (muscle reaction) group with 96% accuracy, and that 98% of the cases (individuals who exhibited muscle reactions) were properly classified. Representative portions of the gene sequences, including the SNP position, used for this classification performance are shown below (SNPs indicated in parentheses; SEQ ID NOS:1-45; “Marker” numbers also indicated, below—compare Tables 1 and 2). The skilled artisan will recognize that the sequences given below can be single or double stranded, even though only one strand is shown. Thus, the invention is contemplated to include the polynucleotide strand shown, the complementary strand, and a double-strand form of the sequence.

TABLE 3
0.37 CUTOFF COLUMN AZ NO INCONCLUSIVES of 94
CLASSED

| | |
|---|---|
| CASE | CONTROL |

| | | | |
|---|---|---|---|
| CASE | 49 | 1 | 0.98 |
| CONTROL | 16 | 26 | 0.619048 |
| | 0.753846 | 0.962963 | 0.815217 |

81% Efficiency
98% Case Sensitivity

2460 LIPMUSC-RS946816

(SEQ ID NO:1)

CCATGAATGAACACCACTGTCAGGTCCAACAAATGCAAATAAAGTAGGAG
CACTGAAATTTGCTAACAAGAAGAGATGATGAACACAGATGGACCCAAAG
TGGTGGAACAAATTTACATAAGTCAATCTATAATGTGTAAGACACATATA
AACAAAGATGAATTATTTCTTTCTACAATAAT(C/T)TGAACTGTAAGTG
GCTTCTAGAACTAGATTCTCAACTCAGCTATATCAAATATTAAATTACTA
AGGGAACCTTTAGAACACCCAGATATTTTAAGTCTCATACAGATGAGCAC
TGTTTACTTAATTTATATTTAAAAATCCAATAAATG
2468 LIPMUSC-RS957928

(SEQ ID NO:2)

GAATTCAGTCTCCATTACCACCTCTTCAGTTGGAGCTCTTACTCTTACCT
GGAAAAATGCAATCTCTACGTTTCTGGTCACTTACAAGTCACATTCCCTC
CACGCATTTCAGCCACATTAACTTTCTAGATCCACAACCCCAATTTACCC
TCTCCCAGAAACTTGCAATGTAATGCAAAATTAATGTCTGTGCTATCGCC
TGCATCTATTCTCTAAATATAACATCA(A/G)TTGACTTTCCACTGTATT
CTCAATTTTCCTCTTTCTATTGAAAGATAATCTCAACCTCTCTCTAATGG
GCAAGTTGAACCTGCCTTTAAGGCCAATCTCCAATGCCGTATTTTTCAAT
AAAACTTCCTGGGTCCTTCTAGCTGATCATAATCTCTCACAGTTTAAATT
TAGCATCACCTATATTCCTCATGGAATTC
2406 LIPMUSC-RS325032

(SEQ ID NO:3)

CTGCGCCTGGCCAGGAAACTTTGCTTTAATAATGTATTGGCTCAGATTGC
AAGTCATTAATTTATTTGTTCAACAAATGACTGTTTTACTACTGCCTTGT
AAGTGCCAGACAAATGTTAGGCATGTGTAGGTTTGGGGACAAGGGGGATA
GATTAGGGATGAAATAATGATCTAGAAGACATCCGATGTGCCATGAAGGA
CCCAACATTTAACAGGCCACAAGAGGAAAGAGACAGACATGTAATAAATG
ATTTTACTTCAACTCATTAAGTGTTTCAATAAAAGTAGACATAAAGTCCT
GAGGTGGCAATTACTAGGGATTGGCCAACTGTGGCTCAAGGAACCAGGAA
GTGCCTTATGGAGGAGCCTAATAATTATGATGGTTCTGCTGTCCTTTGTA
CTATTGTTTAAGATTCATGCCCAATCTAATACCTGATGAC(G/C)TAATC
TTTTTATTGCTTGTGGTTCTCCTTGAGGAAGATGATGAAGAGAGGTGAAA
ACTGCTTCAGCCACCCTCCCCCCGAGGACTGCTGCCCTGCCTTATTTTCC
TTAAGTTTCTGTACCATGCCTCTGACAGAAGAGAAAGTTTATCCCTGGGG
AGGCAAGTGGAGATGAGGCCTAGCATTCCATATGGAGATCCTGGATCCCC
TTCCTTTTGCCCAATATAGACCTGGGGAAGAGTTCAAGGAAGTGGAGAAA
ACAGTACGTTTCCAAAGGCAGAGCTCCTTCATCTGGGAAACTCCCTCCAG
GGGCTGGCTA
2405 LIPMUSC-RS952299

(SEQ ID NO:4)

GGTAAGTGAGTGCATAATGATTCTGCTCCTGCCCATCTCATTCTCTGCAT
TTTCAAAGCAGGGTAAGTATGCACACAGTTCAGAATTACCTCCATAATTA
CACATAAATAAGTAGCCCAGTAATTAAAACATGAACTGCAAAATACCAGG
GTAATCATTATCTTTGGTGATGGAAAGAGAAAATAAGATTGTACGTGGAG
ACTTCAGCTCTCTGTAATGATTTGTTGTTGTTTTTCTAAGAGCTGAAATG
ATAGCATTTGTTAAATTTAGATGACGAGTACACAGGTGTTGATGTATTCC
ATACTTTCCTCAATATTTGAACTATTTCNNNNNNNNNNNNNNNNNNNNNN
NGACATGCAGTGTCTCTCCATTCTCTCGTCAGCACACAGGAATCTGGCGC
CCTCACAAGTTCACACAACAGAAAAACAGCAGGAA(G/C)AAAATGACTT
TCAACCAAGACCAAAGGACAAAGTGAGGGGCGGCAAGATCCCAGCAGGAA
GGGCAACAGGGTCTAGA
2409 LIPMUSC-RS612101

SEQ ID NO:5)

CTTTTTGAATGGCAGGTGCTCTTGCCTGATTAACATTTATTATAGTCAGT
AACTGCAGGAAAGACTGTACACATAACACACGGAGTTATAGGATGTTGGT
GATGTTGGTGTTGGAATGCACCTGTGTAGGATGGTAACTGTCTTTTGCAT
CATGGTAGGAGATTTGGAGACGGAGTCTTACCTAGTTCAAGCCTGTGGTC
CCACCAACTATTAAACCAGTTTAGTAGCTTGACATCTTAGTCTATTTCCT
CACCTATGAAATTCCTACCTCATGGGGTGATTATAGGGAGCAATTAAGAA
GAAGGCGTGAAAATATTTTATGAACTATAAAGCAGGAATACCGGNNNNNN
NNNNNNNNNNNNNNNNNNNNNNNNNNNNNNNNNNNNNCCTGAGGCTGAGA
GATTTGAAGCTT(G/C)TTTTGTTCAGAGTTTTCTAAATCCTAGTTTGAT
CTCTTTTGCTAAATCACACACTCTTGGACAACAGGGACTCATTTTATTCT
CACATCCCTCACAGCAGCTAACGTGAAAATGTTGTTCTTTATTAGAGACA
GCTCCTATCTCATGGATGCACTACAATTACTTTCCATGACTGGCCCTAGG
TTAAGAGAGAATTCCAGGCCAAAGACCTATCTAGA
2432 LIPMUSC-RS967776

(SEQ ID NO:6)

ATAATGCTAGACGGAAGANAATGTAGAGGATTTTGTGGTGGATCTGATGG
TGAGTGGTTAGAGAAGTATTAAGGATGACTTGAATTTTACCCAGAATAAT
TGTTTATACCATTCTTTAAAATCATGAGTTTTGTTTTGGGCATATTAATT
TGCAGATAAACACAAATCCATCAGCAAGATTTAACATAAATCATATTTGT
AGAGGGAGAGTCAGTGAGAAGCAATGAGGGCTTGTGTCTGAGCT(A/G)T
GAGAAATCCCGGTCTTTTAATGGTCTAGATAGTCAGATGGCCTGAAAATG
AGGCAAAACAGGAATAGCTAGAGTTGAGAAAGAACATCCAGGACACTATT
AGAAGCCCAGTTAGAAGATGAGGGAAGAGAGTG
2419 LIPMUSC-RS1384739

(SEQ ID NO:7)

GAGTGGGACACATTTGCCTTCTTTCTTCCCAAAGCACAAGCCAGCACAGG
CCCCATCATCTCCAGAATCAAGTCCAAGTTGCCTTTGTTTGATTTTCAGA
GTCTCTTGGCCTAGTATCAGTCTGCTTCTTAGCTTTACCTGTCAGTGCTA
CACTCTCCTCCTATACTCACCCACCCCACCCAACCTTTGTTCTGGTCACA
CCCAAGCGGTTCAAGGCCCCCTAGCTGGCCCTTCTTCATCTTCGCTGAAA
TGGCATTCCAGG(C/T)TAGCTCTGTTACAGCAGTCATTGTGTACTGGGA
AGTGGCAGGATACAAGATG
2450 LIPMUSC-RS1416527

(SEQ ID NO:8)

GAGGGTCTTCCTACCCAGGTGGAGTCTGTCTTTGAGCCTGTTAACTCACT
ATCCCAGTTTATAAGCAGCCCTTTTTTATGGTAGGCCCCACGTGGTTGGG
TAACCTAACTGATGTGATAATGAGGACAGACTAAAGGTCACGAGATTGTT
AACA(C/T)GTGTGAGGAGATTGTAGGCTTAGCCTATCAGGTGAGGGAAA
GGTTAGCGTACCAACTGTCTCCACACTTCAAAGCATGTAGCTAGTTTGCT
GCACTAACTTAATTATCTGTGCTATTGATATCTTTGAGGAACATATCTAT
ACCCTGGATACAGTTACTTCACAGTAGGAGATAGCAGGAGGTGGCTTGTT
CGGTTGCCAACTTCCTAAAATGCTTTTTAGAGTCAGCTGGGTTGCTTAGT
CCTCTCTTGCTCTTGGATTAAATATAAAAATAACTATGTAAAGCATATCA
GCCCTCTTTTCCCTGTCTGCTGAA
2443 LIPMUSC-RS2200257

(SEQ ID NO:9)

TACAGAACTACAATATTGCAGTCAGGTTTTTTTGTGTTGTGAGATGGCCA
CAAGTTTACAAAGCATCATATATATATGAATTCCNNNNNNNNNNNNNNNN
NNNNNNNNNNNNNNNNNNNNNNNNNNNNNNNNNNNNNNNNNNNNTCTCCA
TATAGTCTTCCCAATCTACGTTTTGAGAGGCCAGGCAGAAAAAAGATGAG
AAAATAAAGGATGTGTATTTAATTAGTCTCTAATCCAATGCTGGACATTT
GCCCAGTAACTTCCCATGCCTCCTCTACCCTTAATCCAGCCTCTCTCTCT
TCTAGAATGACCCTTATCTTCTGGAGACATTCGCACCAGCATGGCCCTGT
GAAATCTCAAATGTGCCTTGGTTTGGGTAAAGGAAAGCAGCCTTGCTAGG
CTGCCTAACACTTTTTCCAGGATAAAGACAGAATCAGATGTCCCACAAGG
GGCCAGCCCAAAATCCATTTAAGTACCTCAATAAATTCTA(C/T)GGCAC
ATCAGTCTGTGAAGCTATATAAAACATATTTAACTCTGTTTTATGACCTA
TTTTACTACTTTTGAGAGCCTTTCCAAACTTTAGAATCACTGTTTTCTCC
AGGAGGAAGGCGCTGATTGGCCCCCTGCAGTCCTGGAAGGCCATTATGGA
GACTGGAATTGGTGTGCTAAAGTCATTATGATTATCATGAATCCTCTATT
TCAACAC
2433 LIPMUSC-RS588037

(SEQ ID NO:10)

TGTACTTCATGCACTGCTCCATCCTCAATGTTTAGATGTATGAATCCAGG
CACTTTTTCTCAA(C/T)GTATCAATAACTTTGCTGAATAATAAGTGTAC
ACAGTGGGACAGGGTAGGCATAAGGTACTGTGGGAGGAAACAAAGATGAC
TCTCATTTTCTGTCTTCAACCAGCGTTTGATCTGGTGGAAGAATTTACAA
TCAAGATGATACTTTCTATTTTACCCTTGCTTTATCTATCATTTCTAGAG
CAATAAAAATAATAACAAGTATGTTTTTAAAAGGAGTCTATTTAGAGCTC
TAGTACACATCAGGTTGCAGTTTTCCATCTGTCAACTGCAGAAACAAATC
CTCTAAAATATAGTGAAATAAAGAAATGTGATGGATAAAAGAAGTGGAAA
ACATTGGCGAAAACAGTACTTTTTTGTCACAAAATGTCAGTGATCACAAA
CTCAGAGGAAATTAATGGAGAAGGTCTACGTTATTTTTTCTTAAAAAATT
GAAGTCAGAATAAGCCACACATTGCTTACAAGGAATTTCAAAGTAAAAAG
TAGGCCTGGCAGCCAGAGTTTGCCCACTGCTCCAAGTGCTATTACAGTTT
ACTCTTCCCTAT
2445 LIPMUSC-RS780373

SEQ ID NO: 11)

TAAGTAAGTAAAGCCTGAAACTCTTAAAAAATTATCTTTGGCTATTTAGA
TTATACTTTTCTGAAAAGGACTCTGTGGTAATGAATCATGTGGTTTCAAG
ATTCACATGCGTTAAAATTACTTCAGGACACTAGTCATTTTTCAATTACC
AGTAGAAGTAAATCTCAAAGGAAGAGTTCTGCAGATTCTTGAGCCATCAA
TGGCCTGTAGGAAATCTTTTGAACAACTGGGTACATGTTGGAGAAAGTAG
GGTCTTGTTAAAACATTTTAAAAATAACTAAACTAAGCCAAAAATCCATT
TTGGATCCATGGGGTAGACTGTCTCATAGAATGTTTCTAAAAGGAGGTTT
TTAAAGTGTTGTTGGATGGTCCCTCTTTTGAGTTG(C/T)TGGAACATAA
ATCATTTGAGACATTTGATTTGATGGTTTATAAAGTTTCCATGACTCTGT
GGTTTATTATTCCCAATTTAGGCACAGGGAGTTGGTTTATATTTATCTAA
TCCAGGTAATAGAGTATTTAATTTCCTGTC
2425 LIPMUSC-RS1343850

(SEQ ID NO:12)

TTTGTTTCTGCATGCAATCCAGCCGACAGCAACTTTTCNTGAGCTATCTC
CATTTCCTCCNNNNNNNNNNNNNNNNNNNNNNNNNNNNNNNNNNNNNNNN
NNNNNACAAAATTTATGAGCACCTGCTGTATGTTGGCATTGCACATAC
(A/G)CATTGGATATAACATTTAAACTACTCTTTTGATCCTTTTAATTGA
AAATGTGATGAAATAATTATTTAAAAGAATTTTTAAAGACTCCACTTTCC
ACTATTTTTAATGACTATTTTTTATCTTACCTTTCAGATTTTACCCATGT
GCATATTTTAAAATACTGAATTCAAATGACAAAACCCTTCATGNNNNNNN
NNNNNNNNNNNNNNNGATACTTATTACTACCTCATATTGTAATTTATCTT
TTACTACATATTAAGCCTTTACATTCCAGTTTTAAACTCTTGCTGCTGAA
GAGAATGAAATTGATATGGATGAAATGTGACTAGATAGAATGAAATAAAT
AGATGTGTTAAG
2407 LIPMUSC-RS1516238

(SEQ ID NO:13)

TTGCACCTGCATCAAACCCCATTGAGAGAACAGATTCAAGAGAGGTGTAA
AGTGTCAAGCCTCAGGACTGCACCCAGAGCATGCTAACTTCCTAGAAGCA
GGAGTAGGGAGAGATTCAGTAAGTAAATCTGAGAAAAAAAATAAAAATAG
GCTAGAGCTAGGGCTACCCACAGGTAATGATGTTTACCCCAACTCCACCA
ATTATTTACCACCCCTTTCCCATATGGGCAGAGGAAAGGATTGGGGTGGA
GAGAGAGACTGGGAATATTCCTTCATTATTGTGTTGGCAAAGACATTGAA
GTTTTCTCCCCTTTCTAGAAACAGGATAAAAGACTATCAACACCTATGTA
AGAAAGACCTACTGTTTTCAGAGTCAGTGAGTCAT(G/C)CAATGCCCTA
TAACTGCCACCCTCACCCACTCAGTGGAAGAAAAAATAACTAAAACATTA
AGCTCTTGCCTTTAATTTCCACTCTTCACCACTGATTAAAACCAGCCATT
AAAAACGGCTGGAGGAGAGAAAGTCTGACTCTTTTTTTGTTGTT
2437 LIPMUSC-rs953004

(SEQ ID NO:14)

TTTATTACCAAGATGATAGAAACCACGGAAAGTTAACAGACAGAAAAACA
GCTCATGGTAC(C/T)ATTGCAATCAAACTCTGACACATTGGGTATAATG
CCTAAAATTGAGAGGAGAAGAAACCTCATAAAGACTAAAACTCACCTTCC
CTCCCCCTTTCAGCTTGAAATAATTTAGCATTTCCTGCTGGCTTTCTTTG
AGGGGTGGCTTCCAGGTATAATTCCTCAAACAGGGAATATTTTTAAATGT
ATCCATATCACAACTTCAATCACTATGTTAAGTGAAGATTCAGCACTCTA
GGCCAGCAACGATTTCTAGAAATATTTTGGCATTTCATTCGTAGCTCTCA
GCCAAGTCATCTCTTGTCTCATATAAAAACTTTCTGATGAACCCACCATA
GATAATCACAACGCATTGCACCTATATAACACATAATACGTTTGAAATGA
TTTTTACAAAAACTGTCTAATTTCATCTGACCTAGGTTACGAAGGATATA
TGATGATTCCCATTTTACAG
2418 LIPMUSC-RS879780

(SEQ ID NO:15)

CTGGAATACAACCCAGCTCACTGTACTGAGTCTCCAGTTTTCAAAAAGTT
AACGTTCCGTANNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNTAAG
AAGTGGAGGAACCAGGATTTGAACCCAGTTCTCTGTTCCCCAAGCCCATG
TGATTTCATTACCTGAGTTGTCGGTGTCACGGGATCCCCCAGGTGGGTGG
GTGGCCCAGTCATTGTGAATCATTTCAACATTTCTTGTTCATAATGTGTT
GTATGAGCCCACCCTTGGCTTTGGCAGACTTCCTGT(C/T)GATGGGCCA
AACGGTGTTGCCCGGCTAAGAAGGTGGTGTTCTGGTTTACGCCACAGGTT
ATTCTCAGAACACAAAAGTGTGATTTCAATCTTCTGAAAATGACTTCGTG
TAGATGCTTTATTTTATTCTACTTGGATATGCCTGTTCGTGGGGATGACT
GTGCCGACATTCCTTTCAACAGCACAACTGAGATGTTTATAGAGAAAGAT
GAGTTTTCTAGAAGTTTAACTGGCTTTTGCTTTTGTACTGAACTGTTATG
AGTTGGGACAATTTAAACATTAACTTTGGTGTCTTGAATTCACTTGTTTA
TGTTAAGAAAACGTGGGGGCTATACCACCCAAAAATTTATTATAACAGTC
TGGA
2394 LIPMUSC-RS1825935

(SEQ ID NO:16)

TAGATTTACAATACATATTCATGCCTCATTCTGGAAATTTCCGTCATTAC
T(A/C)TCTTTCCTCCCAAATGTTTTTGACTGCTCTCACATGTTTTCTCT
TCCAGGTTGACTATAGAATTATTTTTGCTGATTGAGTTTTTCTNNNNNNN
NNNNNNNNNNNNNNNNNNNNNNNNNNNNNNNNNNNNNNNNNNNNNNNNNN
NNNNNNNNNNNNNNNNNNNNNNNNNNNNNATAGAGGCATACAATCATCTA
TCC
2472 LIPMUSC-RS1323434

(SEQ ID NO:17)

CACTGCCGTCTTCCTCACTCTGTATGAATGGAAGAGTCCATTTGGTTTGA
CTTCCAAGGGGCGAAATAGAAGTAAAGTCTTCTCCTTTTCTTCAGCCTTG
AACATCTGTTATGCCCTCTTGTTTGGCAGAACAGTGGCCATCAAACCTCC
AAAATGTTGGACTGGAAGGTTTCTAATGAACCTTGGGCCATTTTCTGTAT
GTTTTGCCTTTCCACATACACGGCAAACTTGGCTGCTGTCATGGTAGGTG
AGAAGATCTATGAAGAGCTTTCTGGAATACATGACCCCAAGGTAATACTT
CATTTTACTTTAGCTTTCTTGATTGTCCATTATAATTCC(A/G)TATGTT
GTATCTTCTGCTGTAGTATGCTCATGTTCTTCCATCTAACACAGGAATAT
TCTCTCAGCCAAGTATAGAGACTAGTCCAAAAGTCTGTTGCCTGGTTTAA
CTAAATATTCCATTGTTTGTTTCATAAACGAAACAAAAAGACTGAGAAGT
TTTGGGGAGTGTCTTTTCTAGAGTAGGTCTTTCTGATAGAAATATCTATT
AATGCATCTTTTCCTTGTATTATTTGACCATCTGATAGATAGCACACCTA
TCAGGGAATGGTCTT
2435 LIPMUSC-RS2007470

(SEQ ID NO:18)

GCATAAAAGCATTTATGTTCTGTACAAAAAGATCAGACTCCTAACCAGAT
TTAGAGATGCAAATATTTGTACCTAAAGAGAAAGACTTTTTCACCTAAAG
TATAGTTTAGGGCTAGTCATCCAACCCTAAAATGTAGGCCACACCTACTT
CAAGAAACATATTCTTGATTCTAGAGAATAAAAAATGTAACCCCACCTGT
CTGTTCTGGCAGGCTCTGGTCTCTGGAACGCTCCATGATGCAGCTAGGGG
AATATAAGCCAGTTGGCATCCCTGCCTGAGGGTGGTGAGATTAGGCGAAA
GATACTTGCACCAAGCCTCCAGGTCTTTCCTCTGCTTACTGTGGTTCTTC
ATCACAAGGGGAGACAAGTCCTACCTCATTGGCATGCCTCACTTTAAGCA
AAGCATAAATGAGAATATAAGCATTTAAAT(A/G)AAGTGCAGATAGGAA
CATTCCTTTTTACCCTATAAAAAAATGCCATCTGACCTACTATGCCATAA
GAGCATAGTAAAAATACTTTGTTTTGAATAGTTTCAACTAGGCAATTTGT
GAGCATATAGCAAAATGACTATTGAGTTTCAGTTGTGCAAATTTGGAAAA
TCTTACATACATTATGGAAATTAGATTTCTTTCCTTTGACAAATGTATTT
TGTGTACTTTCAAAACGTGTGACATTTTAAGTGTTTGAGAAATGGCACAC
TTGCACACCTTGTTAAAGACTAAAACAGCAACATCTGTGGTCAATTCTGC
CCTATATTAGACAG
2463 LIPMUSC-RS1478838

(SEQ ID NO:19)

AGGTTGTGTTTGACCATTCAGGGCTTTCTCTTGGCCTTAAAAATTCTCAG
TAGTAGTAGTAACTCTCTTCCCTGACGACTTATTTGGTTCTATCATTTCA
AATATGAACATATCAAAAATTCAAGAGAAAAGGGATGAGAAATAAATGAT
TTGTCATGGCACAAAAGAAGGTACAAAATGATGATGTCCAAGACGTAGTG
ACAGTTCTTAATCTCTGTAACAGCAGAGAGGTAGTGAAGAAATAATCATA
CTCAATTGCAACAAAGGGAATTTGATGAAATAACAAGGCCATTTGCAAAG
AATGAGTAGATCAATGGTAGGGCCAGGGATTTGAAAAAAGATTTTAAAAT
GTTTTGAGAAAACTCACACTCTTAATTTTTAGAACTTCAATTTCTTTGTT
TAAAATAAAATCTATCCTTCTTCTTCTTCCAATAACTTGATATTCCATCA
TTACAGAATGTATGAAAGTTTTCTAATGGATTCATTTTTCATGCTCTAGA
AGAAGAAATGATACTATACAATCAACTTACATTGCCTTATCTACTTCCTC
TC(A/G)GTCTCAATTTTAGGCATATGGACTGAACTTGGCACTAGTTTTC
TGTCTGGGCTCAGCTACTTACTATAAAAACTGAAGTAATGTCCTCCATGT
TTGATATATGGTAACTAGTCCTGAAGTGACCAACATAATACAAATGGAAA
GTAACTAACATTATATGTCCTAGTGTTTCCAGTGTGATCTTTCTAAACTT
CTATTGTGTTTCTTAACATTTTTTATTGCTCCCTTTTTCCTTGGATAAAA
TATACATGCTTAGGCATGAAATATTAGACCTATCATTTTCACTAGCCTTA
TCTCATCACTCTTGTTACATTTTCTTAAGCAGACTACAGTGTGCTATTTG
TAAATCACAGATTATAACTTGATATATATTTTATTGTCAAGCCATCAGAT
ATGTGCATTTACTTGACATGTTACTCA
2426 LIPMUSC-RS763354

(SEQ ID NO:20)

TCTTGCCTTTAGGTGTGTTTTACCAAAGGAACAAGACAGAAGATGAATGC
CAAAACTATTAATGCAGCTACCCTGGACACTAAGCCCCCAAAGAGATGGA
GGATTGGAGGATGTAGAGCCTGGGCACTGGGTCCAGTGCTGACATGGAAC
ATGTTCATTCTTAGAGTTTCACTGCAAACACTCCAAGGATAGAGAGCGAG
AAGGTTATGTGGACATGAGAGGAGAAGGGTGGAAGAGGATTGAATGGTTC
AGGGTAGCTAAAAAAAATTTTTACACTTTATCTCGGGCTGCTTCTAAACC
CTCCACCAGAAAGAATTAATCCTGCAAGCACTTAAAACAAAACTGATGTG
AGGAAGATAATAAAACCTGTCCAGGGTACAGACATCCAGATTTGCAATTT
AGGGTGGGAAGACTTTAGAGACCTTGTCTAACAGGGCTAAAGAGTATCCT
TATGGTTTTAAAGTAGGTACATTGTTTTTTGTAAGCATTTCTAGGGTTAT
ATAAGACAAATCAGAGTCTGATGTATTGAACACAATTG(A/G)ATCTTAT
TCTGCCATATTTGTTTTTAATTCACGATTTTTAAATGGATTTACTGGTCA
TATTTCTCAGGTCATGGCAAGATTTTTTAGTCTTAAACTTATTTAGCTCA
GTCC
2457 LIPMUSC-RS1561350

(SEQ ID NO:21)

AATTTCTTCAGAATATTCTCTCCTTAAAGGAAAATAGAAAGGAACTTTAC
TTCAGAGCAGAAATCTTAGATTAATAGGATAAATTGTGTATTAGATATAG
AAAGAAAAAATAGTCATTTTGCAACCAAATAATAAAGTGCCACCATAAAA
ACCTACAACACATAATAAGAATAATAAAAAGAACGAGTTATAAAGAGAAT
CTTCATCAACATTAGAGTAAATGGCATCTAAGCAAATCTGAGTTTAGTAT
CATAGTGTTGGAAAGAAAGAACAAAACAATTACAAACATAACAGTATGGA
TTGTTGTTGTATTTAAAGAAGCGGCCCCTGCTAGTACTTCTAGAATGTAG
AAAGATGAAG(A/G)TTCAGGCATTACCTGCTTCCCAGCTGGTTGGATGA
TTATCCAGCTTAGCATCAGGTAGTTAAGTTGTACTGAGGCAAAATGAAAA
CATGAGAGGATAATGCTAGAGGATCCAGCTGGGCCACAGACAAAGTTTTC
ATTCCTCTATTCACTCNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NGTTAATGCAAAAGAATATCGAGGGTACTCTGATAGAAGTGGGGGGTCAG
TGCTACTTTGAACTTGAATGACTTTTTGAAAAATTCCCTTCCAATTAAGC
AAGGCAGTCTTTTCTATATTCCTA
2404 LIPMUSC-RS1027583

(SEQ ID NO:22)

AGATGTTATTTAATTTTTGTGTTTAATTTAGAGGGATCTCAAGGAAAACG
AAGGTATGTCAGGATGGAGAACAGCACACCATCTTGGGTCTTTGATTGAA
AAGTCATAGCAAGGGTGAGATTACCTGGAGTAGGAATCTGCTAAGGAGGA
CATTCTATCAAAGGATCAGTTTACCTGAAAAATCTCCTTACTGATTAAAA
TTCTTCTTATTCCCTACTTTCTATGTCCTTCTCTAAGCATCACATCTCTA
GATGAGTGCCATTTTTCAAATTCAGTGTGCATAAGTGTCATCTGCAGAAC
TCAATATAAANNNNNNNNNNNNNNNNNNNNNNNNNNNNNNNNNNNNNNNN
NNNNNNNNNNNNNNNNNNNNNNNNNNNNNNNNNNNNNGCTCCTTCTTTGC
AGTTCCTTGCCCTCTATTTGGAAGCTCATGGTGTTTACATTATAAATTCC
TCTTTTCTGGCATCTGACTTTCCCTGTGATGGAGAACAGGGAGTCTGAAT
AGGAAAGCACAGCCTGCCATGAGTACATTTTCAGGCTC(G/C)TTAAGTC
TCATATTCATTTGCCGCAAGGAATGAGAAAAGAAGAGAGAGCAACTAAAG
ACTGCATGTTTCAAACATGGGTGATCATCATTTCTCCTGGGGTAACAGGC
TCCCCTTGGAGAAAGAAATTACAAATTACTAATAGATCATATGGCAGAAA
ATTGCAAATTCCAAGCAAAATAAATATATTCTTCCATTTCTAGAGTTTTA
AATTGGCTATAAGGTAGGAGGGTGCAGTATTAATAGGCAGCTATCAAATA
GAACAGTGGTGTTAAATTACATAATCAATTTAATGTTCTGGGTCAGTTGA
CAAAGAAATCTGTTCTCTCACTGCCACTAATTTAGAGCTTTCAATATCCC
ATTCACAAGTCTTTTTAAAAAACTTTTGGGAAAGATTTTGGTATATCAAA
TTCTTCCATAAACATCTGTAACAGCTGTGTCAGGGATCCCAGACTAGCTG
ACCTTTCTCCTTTTGCAGTCTGAGGGTGGAAATGAGAACATAATCGCCCA
TGATGAGCTGGCTGAGCAAAGTTGGGCCAAGCCATCAAATGCAGCTGTTG
ACTTCCCACAGGATTGATCTCTCTTGCCTCACAGTTCACTTCAGGAGGAA
GGTGTGTAGAATATGAGGCCACATGTCCTGTGTGAGTGTGCATGTGTGTG
TGAATACAAGCACACATGTTCATATCCACA
2465 LIPMUSC-RS719546

(SEQ ID NO:23)

GGGCACACATGGNACANAAGTTATGATCAGGGGCATTACGAAAGTGTTGT
GAGAGAGGTAAAAACAAGGTGACTCATAGAACCGAGCTGTGCCTAGCCGC
ACAAGCAGGGATGCACTGGGCAGAGAGAAGAAGGAGAATGCTGCACACAG
AGAAAATGGCACTGAAGAAGGCAGGGAGGCATGGAAAATCAGGAATGCTC
ACAGGATGGTGAGGTCTTCAAGTTGGGATTAAAGTATATATTTGTTCATT
CTTTCACTCAAAACCATTTTGAAGGGGAGAGAGGGACTATCATGTTCTGA
ACATCATAATGAAAGCTTCTGTGAAAGCCCATGTGACATAACCCAACACA
GTCCTTCCTTGCTGGCCTTACACATTTTACTC(A/G)TTCTTTACTGAGT
TCTTTTCTAAGTACAATGTAATGGTAAAGAGCATGGACTCTAGA
2391 LIPMUSC-RS490618

(SEQ ID NO:24)

TAAATTTGTGGTTCCTTGGTTAACTAATCCAACCTTTCACTGAATCTTTC
TATGGTAACTCTGCTCATCATTTTCTTTCTCTGGTCCCTCCTTTCTCACT
ATATGCTATTGCCTTAGTCTTCTTGATAGTCCAAGAAGCCCAGGGTTTTT
TGTAGCTGAGAGTTGGAATTGGTTTTATAACCCCTTAGGATTGAAGATCA
TCCTCTCAAAGTATTGCACAGAATGTAACACTTTCCAAATGTGGTGTCTA
GCNTGATACTGATATGAGACA(A/T)CCTTACCCCCTGTAATCAAGATAT
TGTATATTTCCTGGCAAGTAAATTTCAAGCCTTTTGGCTCTACAGATTCT
CTGTTTCTTGGTTTAGAGGAAATTTAGCAGGTGATGTGTTATAGTATGGA
GTCACTTATATGTTAAAAGCTACATTGTGACATGGGAGATTTCAGTGATG
AAAAACCGGAAGTGCGAAATCTGCAGTTGGCAACTTGATTAAGGAGAACT
TTCTAGA
2447 LIPMUSC-RS674110

(SEQ ID NO:25)

CAGGGCACCCAGAACATTCACTTCACTTGGCATCAGTATTTTGAGGGACA
CCCCCACCACCGCCCACCTTTGTTGGTTCTTGAGCAGGGGAAATAGGACT
ATGAAATAAAGGGAGGCGATTCTAGCCAATGCATACAGGTCTTCCTACCA
CTGAGATGCTCAAAGGTCTAGAGCAAGGAGTTGGGGGGGGCTCCCGTGGA
GACAGAGAGGGCATAGCCTCAAGACTCATTGCTTGGTCGTAACCCTAGAG
CCTTGGATACAGACCCTAAGCTGTCCAAAAGGGATCTCAAGCCTGCCTGT
GGACACCCTGGAGGGACCCCCAATCCTCCCATACCTATGGGCCAGGCATG
ACCCAGGGCCAGGTCTGTAAATGTCTACACTTTTCACTGGAAGGAGCTAA
CCCTGAGCACAGTGGACATCCCTGTGCAAAGATCAGGA(C/T)GGAAGCA
AGGCTGTCCCCAGCCAGAACAAACACCTGCTCCCCCAGCGTCCCCTTGCC
TATTTTCCCACCCTCCTTCTCACGCTGTTCCCTGGCTGCTCAGGAAAGTC
TGGCCCTGGGTAAAAGTCTCCATTCACACCCTTATCCCTCTCCCCTATCT
GGTCAGAATCTGGGGAACCCTACAAAACCATAATATACTTCCCACAGGGA
TCCCCTACCCTGAAGGAAATAACTCCAGATCTCAAGTGTTTTTCTCCACC
CAACTGGCCTGTCTTGGTGCCTGGAATTTCAGCTTCCTCCCTGCTGTATC
AGATTCCCTGGGAACAAAGTTCTCCTGGAAGTGGGGTGGCCTATGGCTAC
CGTCTGACACCAGCCTAGTGGAGAAAGAGGGTTCAATGAGCTAAAAGGGC
TCCCCACCACCTCCTCTGAGCCATCACACACCCCCACATTGTGCTAGAGT
CTCTGCCAATACCCCCAGCCAGCGCTCAGCTGGCCGC
2392 LIPMUSC-RS717007

(SEQ ID NO:26)

TTGTTTCCATACTTTCAAAAACACTATTTTGTTTACCAATTATCAAGCAT
TTGTTAAGTATCTATCCCAAGCTAAGTACTGTGCTATATTGGTCAGGTCA
GGCAACGCAGCACCTTAAAAGTTGAAGAGAAGCCTTTGGAACCATTATTC
ATTCATTCAATTGAATATGGTCAATTGGCATCATGAATCTAACAATTAGG
AATTGGGTAGCAATATGAAGGGTGTGTATGGGAAGGATCAGGTGGCAGGA
GAA(A/C)AGCAAGAAGACAGGATTGGGGTCATTTTCAGTCCTGGGCAAA
GAATTGAGCATGCAGACAAGATCAGTGGGGTCCTTCCTAGGTATTTATGA
ATCCCTGAATCTCAAACTGAATACCTTGTGTAGCAGAAGCGAGCGTACAC
AGACCCTAGCAGATACACATGTGAACCTGGGTAGTCCCTGAAGAACAGGA
CAGGGCTCAGCTGCAGGTACCGTGCACTATGACCCTGCCAGCCACACACA
TGGGCACTAACATTCTGAGCAGTCA
2455 LIPMUSC-RS967326

(SEQ ID NO:27)

AGTCTCCCAGCCTTGAACTTTCCATTTCAAGATAACCTTTCAGGCCTTAA
TGTAGTCTGAAGAGATATGTGTTCGTGGAATATTTAGCTTTCAAGAAACA
AATAGCATAGGAGGAAAAAAAGAAACCTCAATATTGGTAATTAAGAAANN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNGATTGA
AGATGATTCTTGCTGAGGCCTCTCCCAGCTCTAATAATAAGTCCAAATGA
ATAGTCTAGGAAACAATTAATCATTAGCATCTTTATAACAGCAGCACTAG
CCTCTTGAATATGTTGGGC(C/T)ACGTGACAAACATGGACAGAAAATCT
TGGCAAGCCCCTGCCCTGTCACTCCCAAATCAGCCTCTGTCACTTTACTC
TGTTTCAGTACAAGCAAATGTAATGTGAGGAACCTAGGGAAAGTCATGAG
AGCCTTTTGAATTAACTGAGTGTTACAACCGCCATTCTGTTCTGAAAATA
GGCTGAGGCTGAGTTCCTCTTGACACCTCATGCCTCTCTTCCCTCCCCAG
TCCTTCTTGGTATCC
179 CYP2D6_RS1058174

(SEQ ID NO:28)

GTCATCGGTGCTGAAGGATGAGGCCGTCTGGGAGAAGCCCTTCCGCTTCC
ACCCCGAACACTTCCTGGATGCCCAGGGCCACTTTGTGAAGCCGGAGGCC
TTCCTGCCTTTCTCAGCAGGTGCCTGTGGGGAGCCCGGCTCCCTGTCCCC
TTCCGTGGAGTCTTGCAGGGGTATCACCCAGGAGCCAGGCTCACTGACGC
CCCTCCCCTCCCCACAGGCCGCCGTGCATGCCTCGGGGAGCCCCTGGCCC
GCATGGAGCTCTTCCTCTTCTTCACCTCCCTGCTGCAGCACTTCAGCTTC
TCGGTGCCCACTGGACAGCCCCGGCCCAGCCACCATGGTGTCTTTGCTTT
CCTGGTGAGCCCATCCCCCTATGAGCTTTGTGCTGTGCCCCGCTAGAATG
GGGTACCTAGTCCCCAGCCTG(C/T)TCCCTAGCCAGAGGCTCTAATGTA
CAATAAAGCAATGTGGTAGTTCCAACTCGGGTCCCCTGCTCACGCCCTCG
TTGGGATCATCCTCCTCAGGGCAACCCCACCCCTGCCTCATTCCTGCTTA
CCCCACCGCCTGGCCGCATTTGAGACAGGGGTACGTTGAGGCTGAGCAGA
TGTCAGTTACCCTTGCCCATAATCCCATGTCCCCCACTGACCCAACTCTG
ACTGCCCAGATTGGTGACAAGGACTACATTGTCCTGGCATGTGGGGAAGG
GGCCAGAATGGGCTGACTAGAGGTGTCAGTCAGCCCTGGATGTGGTGGAG
AGGGCAGGACTCAGCCTGGAGGCCCATATTTCAGGCCTAACTCAGCCCAC
CCCACATCAGGGACAGCAGTCCTGCCAGCACCATCACAACAGTCACCTCC
CTTCATATATGACACCCCAAAACGGAAGACAAATCATGGCGTCAGGGAGC
TATATGCCAGGGCTACCTACCTCCCAGGGCTCAGTCGGCAGGTGCCAGAA
CGTTCCCTGGGAAGGCCCCATGGAAGCCCAGGACTGAGCCACCACCCTCA
GCCTCGTCACCTCACCACAGGACTGGCTACCTCTCTGGGCCCTC
391 CYP2C8_RS1934952

(SEQ ID NO:29)

CTTGCTTTTCACACCTGTGTTTTGTGTGCTAAGTCTCTGGCCATGGAATC
TCCTTCCTTTATTCTTTGATCTTTTATGTCTGCTTTATATTTGGCACTGT
AGATACCAACATGACAAGGACAGAGACCTTCCTTCAAGAATTCACATTTT
ACCACAATAGATAAATAAATACAGAATTACGATTCCTGGTAATCTGTGCT
TTAATGAAAGCATGAGTATGCTGTTGAGGAAAACCAGAGAGCATAGGATT
TGATTACTTGAGGGGATTGTAAAGAGTGTGAAGATGAAATGAAATTGAAC
CTATGTCCTGAATGTAGCCTAATAAAGATGTATCTTAGCCAAAATGAACA
ACAGGATATGAAACTTGAAACTTCATGGTGTGTTCAAGATTGATGAAGAG
AGTGTATGACCAGAGCTGA(A/G)AACAGATTATGTGAAAAGTGGAAGGA
AAAGTCATGGAATATCAGGCTTGGAACAGGAAACAAGAATGTGCATGTAT
TTCCATAATGACAGATGGAAACTCAAAATGGCAAAAGAGCTGTTTTAATG
TGGGAATAAATAA
2689 HMGCR_41344_712043

(SEQ ID NO:30)

AACAAAAGGCCTCTCTGAACAAATAGCCTGCGGAGATAAATACAGTGATT
TGTTTTCCTGATAGAACTATTTAGCATGTTTAACACATTATTCTGTAGTT
TGGGAATAAGAGTGTTTCTTCCCTTGAAGAAAACAGGTCCCCTTCTGAAG
AATAATGCTGATTACCCCCCAAAATCAAAATAGACCAGCACCAAATGAAG
TATTAATTTACAAACATGAACTTAGAACTTAGCTCTTACTTCTTGAAGTT
CTACATCCCAGACTTAATAAATTAACTACAAAATCAGGAGTTTCATCAGC
TACAGTATAATTTAAAAATCCATTTTCAACTGGCAGGRGTGAGGGAGAAG
GTCAATTGCACTGATCACCATGAACTTCAAGAATTTCATCAAAACTTTTT
TCCCAGCTTATATTTGCCTTCAGAGGTGAGCTGTAGATTACCATCTCTGA
TGCTTTAACATACAATATTCTTGTTGAAATCTCTTCAAAGAGCACAGCAT
GTAAAGCACTAAACTGTGTTCAGATCTGAGGAGTCTGCATGGAAAGAACC
TGAGACCTCTCTGAAAGAGCCAAAAACCAAGTGGCTGTCTCAGTGATCAC
ATCTATTCATCCTCCACAAGACAATGCATTGAGCTTTTTTAATTCACAGA
TTTTATGTTAGTCCTTTAGAACCCAATGCCCATGTTCCAGTTCAGAACTG
T(C/T)GGGCTATTCAGGCTGTCTTCTTGGTGCAAGCTCCTTGGAGGTCT
TGTAAATTGATCTTCGACCTATGGTAGAAAATGACAAAGTAGCAATATAT
AAATATCAGGAGTGTAGAATTTTAACTTGGAACTACAGTAGATGAATAGT
AAGTTTTTACACTGCATATTTTTTGAAGTATAGGGGGAACATGTTAAATA
TATCTTTGAGTCTTACCTGTTGTGAATCATGTGACTTTTGACAAGATGTC
CTGCTGCCAATGCTGCCATAAGTGACAATTCCCCAGCCATTACGGTCCCA
CACACAATTCGGGCAAGCTGCCGGGCATTTTCCCCAGGATTATCTTTGCA
TGCTCCTTGAACACCTAGCATCTGTAGCCAGGGAGAGACACAACAAGATT
CACCCTTAAAATCATGACCAATTTCTTACTAAATCAACTAAAAACAGGGC
AACTGTAATGGCATCAGAATAGAACTAGACTCCACTGGAAGCACTAACTT
TCCAAGACTTGACAGCCACACCTGACAGTGCATAATACCATAGCTAACAT
AATATTCACAGCCTGACTGGCAGTACCCTTAACTCAGTAGATGAACATTC
ATTTGCTCTCTTCATCTACTTTCTTATCTAAGCATAAGCTTAAACATGCT
TATTTGGACACAATGGATTAGGCTGATATGACAAAAGAGTTTGGAAAAGA
CCAATTAAAATAGAGGTGAGTGATACATAGTCTCAGATAGAAAGAGAAAC
CCAGAGAGTCAGAACTAGGCTTGTGGACTCTATGCCTGATACATCATACC
TG
2760 CYP2C8_2185571

(SEQ ID NO:31)

TTAGGACCTATAGATGTGAATTTGGGCACCCTTTCCATTTATGTTCACTA
GTCACTTGGTTACAGCAATTTCATTTTAGAGATTCCTTCACCGAATAAAA
GCCTTTTTAATAATTTCTTTACCTGTAAAATTACAGATTTGGCCTAACTT
ATGGTCAAATTTAAAACTTTATTAAAACATACTTATCTTGATGACTATAC
TGAATAATATCCCAGAATCCACAAGAGTAGTTTTGCCCAAATGAATTAAA
TTTGGAAGATGATTGGAAATGATAAACATTATTTCCAAAGGAAGGAAATT
CATTTTTTACAAATTGAAAAAATAAATAAAAAGATTCCATTTATCAATTT
ACTTTTTGGTTAGCTTTACTGGGATTTAGATTTGAAATAAAAAGTTAGCA
TTTTACTTATTTAGATAAACAAATCTGTTATACTTAATGCTCATTTATAT
TAGTTTTTCTTCTCATATCATATGGATGTACTTGCTTTTCACATTTGTTC
CCTAAAACACATGAATTCACCCACAATCTCCCTAACTTTATTCCCATTGG
TTCAGCACATTATACTTTCAGCTATATGACATTTCTAGGTCCCCAACTTT
TCTCTTCCTATCTCTGTGTTTTATTGTCCAGAGGCTTTGCAAACAGACAT
GATTCTGCTCTAAAAACTTGAATTATTAGGATTTCAAAAAAGTCTTCTTT
TTCCTTCCAAAATAAAAGACACTTGG(A/G)GTTAAAAGAGTTTAAATAA
AGGCAGTGGATGTGAATAACCACATGCGAAATGACATTGTTTAGCTAGCC
TGTAATGCTCAACTCATATTTAAGGTAAAAGTAATGTGTTTATTTCATCC
ATGCTGATTTTTTTTGGACACATGGGGAATTTGTAAGATATTGTTTAAAA
TTTCTAAATTTCCTTTATGTCTTAACAAATGCAAATC
381 CYP2C9RS2253635

(SEQ ID NO:32)

CTCACAGCAGTACTCCTAAAATTAGATGGCTATAGGATTATACAAATCAC
CTTAATTCTTAACCCCCTAACAGAGTTTATAGCTTATTCCTCCTTCTTAC
TATCCTTTTGAGAAATAATCATAACAAGCTCCTTTTGTCTGTGCCAAACT
GATCTAAAATCACTCATTACTTTTCCATAAGCCACATGGCACTTGTTATC
(A/G)TAGCTTTTCTCATTCAAACCCCCTGAAGTTTTACCAGTGCAACTT
CCCTAATAACTGCCCACAGACTAACCTCATCCCTATTATTCTGCCTTGCA
AACTCAAACTACAAACGAGTTCACAGCCAAACCATACTACTAACACAAGG
CCTTCAAACACTTCTCTCCCTGATAGCCATGTGATGACTCCTAGCTAGTC
TTGCC
362 CYP2C8_2071426

(SEQ ID NO:33)

TTTGTCTTTCTAAGTCTGTCATTGTCAGAAATAGCGGAGTGAGTTGATGC
ATTTTGTGAATACAGAAACATTGGGGTCATTGTATTATATAATCATTTAA
TACAGTGGCAAAAGTTTAAAGTGCTGTTTCTCCTCTTTGTTTCACAGTGT
TTTGCTATGATTTTTGACTGAAGGTGAAGGGAAGTGTGTGTGATTAGAAA
TTTCATCCA(A/G)TAAGTTCTCTACTATAGTAGTCATGTGTTTTATTCA
GAATGGTCATGAAAATTGAACTTCTCTGAAGATTCATTTGATGGCTGATG
TGAAATAAATATCTGTGGGTTCAGGGCAAACATAAGTGCATGAAAGAAAG
AAGTAATCAGTCAGGGCCCAATAGGTAGTTAACAGAATTCTTTTGGATTC
TGAAGAAAGCCACTGTCTGTGGCCAAGGTTGCTGGAGAATGGAAGAAATT
GTTCTTCCAGGAGATGCTGAATGTCCTGATTCTAACTTTGTGGTGCTTCA
TCGTTCCATATTGGTAATAC
58 GSTP1E2E4-5_419

(SEQ ID NO:34)

CCGGGCAAGGGGAGGGGGTGCTGGGCCTTAGGGGGCTGTGACTAGGATCG
GGGGACGCCCAAGCTCAGTGCCCCTCCCTGAGCCATGCCTCCCCCAACAG
CTATACGGGCAGCTCCCCAAGTTCCAGGACGGAGACCTCACCCTGTACCA
GTCCAATACCATCCTGCGTCACCTGGGCCGCACCCTTGGTGAGTCTTGAA
(A/C)CTCCAAGTCCAGGGCAGGCATGGGCAAGCCTCTGCCCCCGGAGCC
CTTTTGTTTAAATCAGCTGCCCCGCAGCCCTCTGGAGTGNNNNNNNNNNN
NNNNNNNNNNNNNNNNNNNNNNNNNNNNNNNNNNAGCCTGGGTGCCTGCA
ATCCTTGCCCTGTGCCAGGCTGCCTCCCAGGTGTCAGGTGAGCTCTGAGC
ACCTG
424 GSTP1E2E4-3_466

(SEQ ID NO:35)

AGGTGCTCAGAGCTCACCTGACACCTGGGAGGCAGCCTGGCACAGGGCAA
GGATTGCAGGCACCCAGGCTNNNNNNNNNNNNNNNNNNNNNNNNNNNNNN
NNNNNNNNNNNNNNNCACTCCAGAGGGCTGCGGGGCAGCTGATTTAAACA
AAAGGGCTCCGGGGGCAGAGGCTTGCCCATGCCTGCCCTGGACTTGGAG
(G/T)TTCAAGACTCACCAAGGGTGCGGCCCAGGTGACGCAGGATGGTAT
TGGACTGGTACAGGGTGAGGTCTCCGTCCTGGAACTTGGGGAGCTGCCCG
TATAGCTGTTGGGGGAGGCATGGCTCAGGGAGGGGCACTGAGCTTGGGCG
TCCCCCGATCCTAGTCACAGCCCCCTAAGGCCCAGCACCCCCTCCCCTTG
CCCG
216 CYP2C19E7-5_169

(SEQ ID NO:36)

CACTATAATTTAAATTTATTATGATGTTTGGATACCTTCATCATGATTCA
TGTACCCCTGAATTGCTAGAACAAATGTTCCATTTCTCTCCTTTTCCATC
AGTTCTTACTTGTGTCTTGTCAGCTAAAGTCCAGGAAGAGATTGAACGTG
T(C/T)GTTGGCAGAAACCGGAGCCCCTGCATGCAGGACAGGGGCCACAT
GCCCTACACAGATGCTGTGGTGCACGAGGTCCAGAGATACATCGACCTCA
TCCCCACCAGCCTGCCCCATGCAGTGACCTGTGACGTTAAATTCAGAAAC
TACCTCATTCCCAAGGTAAGTTTGTTTCTCCTACACTGCAACTCCATGTT
CTTTTA
374 CYP2C8_RS1934953

(SEQ ID NO:37)

CTTGCTTTTCACACCTGTGTTTTGTGTGCTAAGTCTCTGGCCATGGAATC
TCCTTCCTTTATTCTTTGATCTTTTATGTCTGCTTTATATTTGGCACTGT
AGATACCAACATGACAAGGACAGAGACCTTCCTTCAAGAATTCACATTTT
ACCACAATAGATAAATAAATACAGAATTACGATTCCTGGTAATCTGTGCT
TTAATGAAAGCATGAGTATGCTGTTGAGGAAAACCAGAGAGCATAGGATT
TGATTACTTGAGGGGATTGTAAAGAGTGTGAAGATGAAATGAAATTGAAC
CTATGTCCTGAATGTAGCCTAATAAAGATGTATCTTAGCCAAAATGAACA
ACAGGATATGAAACTTGAAACTTCATGGTGTGTTCAAGATTGATGAAGAG
AGTGTATGACCAGAGCTGAGAACAGATTATGTGAAAAGTGGAAGGAAAA
(A/G)TCATGGAATATCAGGCTTCGAACAGGAAACAAGAATGTGCATGTA
TTTCCATAATGACAGATGGAAACTCAAAATGGCAAAAGAGCTGTTTTAAT
GTGGGAATAAATAA
172 CYP2D6_RS2267446

(SEQ ID NO:38)

TGTGCGGGAGGACGGGGACAGCATTCAGCACCTACACCAGACAGAACGGG
GTCTCAATCC(C/T)TCCTGTGCTCTGCGTTCATCTGGACCAGTCTCAGG
CCCCAGCCATCTCCAGGAAGACCCA
369 CYP2C8_RS1891071

(SEQ ID NO:39)

TAATCTGTCTAAATTTGATGACACAATTTAAAATGACATCTTTGTACAAT
GGAGGAGGATGACAGAGATCAGTA(A/G)AAACAGTATGGCAGTAGCAAA
ATAAGTAAAGCACTGATGAAGTGTCTGGATTTCAGCAAAGGTAATTTGTG
GTAAGGAGAGCCAGCATAAATTGCCCTAGTATTGAATGTTGGTTTTATTA
TGAAAAGTCCACTTTGAACAGTAGGTTCATTTCTCATTTTAAAAATTCCA
TGCTCTAATGCTGTGGTGGGGAGATGAAAACAATCTTTATTGAAGCATAA
GTGGAAATTCTAGAATTGTACTGAGGCATCCTGACATAAATTCCAGTCTG
GGAAGTAATCTAAAAGTTAGTCTCTTACAAAGGTGTTTCTATTTAAGCAG
AGGCCATACCTAAAGGAATTTTATTATTCTAGGAGTGTGTTTCATAAAAA
TGCTATTTGACCAAATAGGGACATTTGGAAGGGGGTTTAATAATTGCATC
TTTCACATACAACTTTTCTTTAGAATTTACAATTTACCCTTAGAGTAAAC
TCTACATTTCCTTAGAATTTACATTTAAATAGTTCCTGCTTTGCAGCAGA
TAACATACCTTTTTCCCTGTGTCAGGATCCCACTG
282 CYP2C8E8_296

(SEQ ID NO:40)

ATTTCCTAATCCAAATATCTTTGACCCTGGCCACTTTCTAGATAAGAATG
GCAACTTTAAGAAAAGTGACTACTTCATGCCTTTCTCAGCAGGTAATAGA
AACTCGTTTCCATTTGTATTTAAAGGAAAGAGAGAACTTTTTGGAATTAG
TTGGAATTTACATGGCACCTCCTCTGGGGCTGGTAGAATTGCTATTT(A/
G)TCCATGATCAAGAGCACCACTCTTAACACCCATGTGCTCCACCCTCAC
AATACACCATCATTATTGGGCCAGATAGAAGGGCGAATTCGTTTAAACCT
GCAGGACTAG
225 CYP2C8_RS1891073

(SEQ ID NO:41)

TGAGATATGGACTCCTGCTCCTGCTGAAGCACCCAGAGGTCACAGGTAGG
ACCACAGATGATGAACAAAGTGAATTTCAGAACAATGCTGAGAAGATGGT
GCCAGTATCCTCCACCTTGTTTCTCTCAGAGAAGGCTCATTCTTTAAATT
TCTGTGTCATCAGCTGTAATCTGTCTAAATTTGATGACACAATTTAAAAT
GACATCTTTGTACAATGGAGGAGGATGACAGAGATCAGTAGAAACAGTAT
GGCAGTAGCAAAATAAGTAAAGCACTGATGAAGTGTCTGGATTTCAGCAA
AGGTAATTTGTGGTAAGGAGAGCCAGCATAAATTGCCCTAGTATTGAATG
TTGGTTTTATTATGAAAAGTCCACTTTGAACAGTAGGTTCATTTCTCATT
TTAAAAATTCCATGCTCTAATGCTGTGGTGGGGAGATGAAAACAATCTTT
ATTGAAGCATAAGTGGAAATTCTAGAATTGTACTGAGGCATCCTGACATA
AATTCCAGTCTGGGAAGTAATCTAAAAGTTAGTCTCTTACAAAGGTGTTT
CTATTTAAGCAGAGGCCATACCTAAAGGAATTTTATTATTCTAGGAGTGT
GTTTCATAAAAATGCTATTTGACCAAATAGGGACATTTGGAAGGGGGTTT
AATAATTGCATCTTTCACATACAACTTTTCTTTAGAATTTACAATTTACC
(C/T)TTAGAGTAAACTCTACATTTCCTTAGAATTTACATTTAAATAGTT
CCTGCTTTGCAGCAGATAACATACCTTTTTCCCTGTGTCAGGATCCCACT
GAAATTTCAATAACTAAATTACAAATTAAAATTATCCTGCTGAAGTGAAA
AGAGAGGAGAATAGAAAATAGCCAATAAGTCTTTGGCAAATTTCTAAAAC
ATGGAGAGACAATGGAGGGCTGACAATTGACACAGCATAGAGGATGGAGT
TGTAAGTATTAGCAGAGGAAGATTTATCAAGAGGAGAAGCTAATCTGTGT
CACTGTGATTCTCATAAGGTTCAGTATTTGGAGGCACCAAGAGCCAAAGA
CAGTAGGGAATGAGACTGAAAAGGGAGGCTCTCCTGCCCAAGTTTCCTCC
ATAACTCCTTTTATCAGGTACCTGATTTTTTCCCACTGGGAAAGGGAAGC
ATACTCTCCAGAGAGACTGAGCCTAAGAACCCCTGTACTCGGCGACACTT
TACAGGGCCAGGAAACTGTGAGATGTGAAGACAGAAAAAAAAAAGGTTTG
AGAAAGATCTATTCCTCAGATTCAAAGACTCAGCATTTTTCTTCATCTTG
CTTCCTGACATTCAGCAGCCAGCTAGATACTCTCCAGAGAAGAAACAAGT
CTGGAGAAAAGACCTCCGTATTTTGCCAACTTAATAGAAAGTTATCTATA
2680 HMGCRE711_635_756249

(SEQ ID NO:42)

AGATAATTGTACCTACCTCTCAAGGTTGTGTGAAATAAAGAAAGAAGATA
GTATCTGTTAGTCATGTTACACCGTGCCCAGAACTTAGCTGTAGTGTTCA
GCAGATACTAGATTTTTCTTCCTTGAAAAAGCTCCTTTGTAATGAAAAAT
GCCACATTGGAGTTTATGTTATCATCCTCACCTCTGCATTCCCAAGTATC
TGTAGACATTCTTCATTAGGCCGAGGTTCCCTGGGAAGTTCAATTTCAGG
TTCCTGTGTCACCAGTACTGATGAAGTATCGAGTAAGGAGGAGTTACCAA
CCACAAATGTAGCTCTGTTTGGGGTATCTGTTTCAGCCACTAAGGGTTTT
ATAACCTCAACTGTTCAAGAAATCAAAAGAAATCTCACAACACGGCACAT
AATTTTGAAAACAGACTACTTTTTGACAAAGCAATGAAGGATTGAAAGCG
AAGAGAATAACAAGTTACCTTTTCTTTCTCGGTTTATCCCTGTCTCTTCC
TCTACTGAATCACATTTCTGGTTATTTCTGACCAGCATAGGTTCACGTCT
ACAACAATTGTCTGGGACTTTCTTTTGTGTCACTACAGGAGATGTGATAG
GGTTTTTTAATGAGAGTGTAGATTCTGTCTCTGTTTGTTCAAAGAAGATG
TACTTGACAGCCAGAAGGAGAGCTAAACTTAGGGTAATAACTTGTTCAAT
ATCCATGCTGATCATTCTAAATAAAAGAAA(A/G)CAAATTAAAATCTTA
TTCAGAAATGTAAAGGACACAATCTAAACTTACATTAGCATAGAGTGTTA
TGATTAATATATCACAAAGTAGATTTCAATTAACTTACTTAGAGAGATAA
AACTGCCAGAGGGAAACACTTGGTTCAATTCTCTTGGACACATTTTCATC
CAGTCCTAATGAAACCTTAGAAGTATCTGCTGTACTGTTTTGAGGAGAAG
GATCAGCTATCCAGCGACTGTGAGCATGAACAAGAACCAAGCCTAGAGAC
TGAAATAAAATTTTTAAAGTAATGTATCCTCTGCATATCAATAGAACTTA
AATTTCTTATCCCTAGCAACTGGACAGCCAGACATTATCTCTCATAGCTT
CCCCTTACCATGAAAAAAAAAAAGCCCCAAGCTTGCTATGCAACTAACTA
AAGTAGTGACCCCACTGAACTACTGAAAACACCCCAAAGAACAGGCTTTC
AACGAGAGATAAGGTGGGGGAACTTAAAAAGTCTGTTTAGGAGAGAGGGG
CTAATAAAGACCAGGAGCCTCAAAGAAATGAACACATTAAGAAAAAAAGG
AAGAAGGGGGTGCAATATCATTGAATGGGCCAAAATTGTAGAAAAAAAGA
AATCTTAWAAATAATGAGATTGGAACTGAGGATACTAAAAGAAGAAGAAA
ACCATGTCATTACCATAATCATCTTGACCCTCTGAGTTACAGGATTCGGC
TTATTTTCTTCTTCTTCTAAAACTCGGGCAAAATGGCTGAGCTGCCAAAT
TGGACGACCCTCGCGGCTTTCCCGAGAAAGCTACAAATTAAGTC
98 CYP1A2E7_405

(SEQ ID NO:43)

CTAGAGTATACCAGTCCACTCCAGGGAAGATTGGAGCTGAGGCTGCTTGA
GGGCTATACACACTCTGGGAACTAGGGGGTCTCCAAACCCTTGAGAGGTT
TGCAGGAGGAAAACTGCAAGGAGACTGGCAGAAAGCAGGCTGAAGTGGAA
GCTTCCTGGCCCGTGCTGGGCTC(G/C)TCAGTGCTTGAGAACATAGATG
AAGGGCAGACAGTGGCCGCAGACGAGGGACGCTGTGAGGAGGAGGCCTGG
CATGTCTTGGGGCCAGGAAGAGCTCCCTGATCATTTTTTCCTTCAGGATG
GGTAGGCTCTTGGTCTGACTGCCCGGAAAACAGGTGATGGAAGGACCAAG
GACTACAAAGGTCAGGAC
188 CYP2B6RS1042389

(SEQ ID NO:44)

AGACCACAGTCACACACTGCTGTAGTCTTCCCCAGTCCTCATTCCCAGCT
GCCTC(C/T)TCCTACTGCTTCCGTCTATCAAAAAGCCCCCTTGGCCCAG
GTTCCCTGAGCTGTGGGATTCTGCACTGGTGCTTTGGATTCCCTGATATG
TTCCTTCAAATCTGCTGAGAATTAAATAAACATCTCTAAAGCCTGACCTC
CCCACGTCAAGAGGTGATCTGTGCCATTTTGTGTGTGATTCTTTTATTGT
CGGGTCTCTAGGGATTTTTCTGGAAGGAA
2657 HMGCRE5E6_283_809125

(SEQ ID NO:45)

TACCCTGGGTTTCTCTTCCATTAGATCTTGTTCTGCTCTCTGATGTGTTT
CACTAGGAAAATACTCTTATTTACCCAAAAATTATTATTACCATAAGTTC
TGAAAACTTTCAAAAAGAAAAATGGGGGYAATTCCAAATTCCAGTAGCTA
CAGAATCATAATTGAGTTGTTAGATACAGGGGACTGTTCCTGGGGCACTT
ATGGAGACCAGTCTTGGGACTTRAGAATTAAACTTAAAACTTTGGGCAAT
TCTTAAATCTTGTGCTATGAAGAAA(C/T)GCTATTAATCCTTCCTATTA
ATGTAAACTGAAAAAAGGAATACTATTCACATTCCTATCTTATAAATAAT
ACTTACCTGTGAGTTGGAACTGAGGGCAAACTTTGCTAATGTGCTTGCTC
TGGAAAGGTCAATCAAAAGTAGGAAAAAGGGCAAAGCTTCACTGGAAAAG
AACAAAATGATCAGATAAATTTAACGGGAAAAAGTATGATTTNNNNNNNN
NNNNNNNNNNNNNNNNACCTTTCCCCCTCCATACTGTATGATCCTGTAGT
ATGTGTACCTTTCTGCAGACAAAAAAGTATACCCTATATTTCTTTGGCAT
CCTCAAAGCTAAACATAGTAGTTGCTC

### Zocor® Study

A set of 205 xenobiotic metabolism SNPs, including SNPs in the AHR (SEQ ID NO: 125), CYP1A2 (SEQ ID NO: 127), CYP2B6 (SEQ ID NO: 130), CYP2C8 (SEQ ID NO: 131), CYP2C9 (SEQ ID NO: 132), CYP2D6 (SEQ ID NO: 134), CYP3A4 (SEQ ID NO: 136), CYP3A5 (SEQ ID NO: 137), GSTM1 (SEQ ID NO: 141), GSTM3 (SEQ ID NO: 141), HMGCR (SEQ ID NO: 143), MVK (SEQ ID NO: 144), PON1 (SEQ ID NO: 145), PON3 (SEQ ID NO: 146), CYP1A1 (SEQ ID NO: 126), CYP2C19 (SEQ ID NO: 133), CYP3A4 (SEQ ID NO: 136), CYP3A7 (SEQ ID NO: 138), CYP2E1 (SEQ ID NO: 135), CYP1B1 (SEQ ID NO: 128), CYP2A6 (SEQ ID NO: 129), CYP4B11 (SEQ ID NO: 139), and GSTP1 (SEQ ID NO: 142), was screened as described above and in PCT International Publ. No. WO 03/002721 (published Jan. 9, 2003) and U.S. patent application Ser. No. 10/188,359 (published Nov. 20, 2003 as U.S. Patent Publication No. 20030215819 A1), the entire contents of which are incorporated herein by reference. The top SNPs, ranked by delta value are shown in Table 1 (“ZOCORDELTA”). As shown in Table 4, four of the genes screened —CYP2B6, CYP2D6, CYP2C8, CYP3A7 and CYP3A4-showed multiple hits, indicating that the results obtained are not random with respect to gene level of analysis

TABLE 4
ZOCORDELTA

| Sample size | Delta | Marker | |
|---|---|---|---|
| 32 | 0.229167 | 2663 | GSTM1PE1_287_TC |
| 66 | 0.155303 | 324 | CYP2B6RS2279343_GA |
| 68 | 0.145469 | 2672 | CYP2D6_RS2004511_GA |
| 70 | 0.140336 | 65 | CYP1A1_RS2606345_CA |
| 70 | 0.131746 | 225 | CYP2C8_RS1891073_TC |
| 70 | 0.131746 | 249 | CYP2C8_RS1891072_TC |
| 66 | 0.129234 | 74 | CYP2E1RS915907_CA |
| 62 | 0.129032 | 134 | CYP2C8E2E3_397_TC |
| 70 | 0.127027 | 373 | CYP2B6RS2014141_GA |
| 68 | 0.12437 | 172 | CYP2D6_RS2267446_TC |
| 68 | 0.123162 | 179 | CYP2D6_RS1058174_TC |
| 68 | 0.11683 | 2739 | CYP2B6RS1038376_TA |
| 68 | 0.108824 | 93 | CYP2D6_RS2267444_GC |
| 66 | 0.107323 | 98 | CYP1A2E7_405_GC |
| 70 | 0.106767 | 108 | CYP3A7RS2037499_GC |
| 70 | 0.105405 | 2665 | 3A4E10-5_292_712037_GA |
| 70 | 0.104633 | 178 | CYP3A7RS2740562_TC |
| 70 | 0.104633 | 263 | CYP3A7RS2687077_GA |
| 70 | 0.102381 | 96 | CYP3A4_RS2257401_GC |
| 70 | 0.102381 | 229 | CYP3A7RS2687075_TC |
| 70 | 0.102381 | 233 | CYP3A7RS2687076_TC |
| 70 | 0.102381 | 364 | CYP3A7RS2687143_GA |
| 70 | 0.101587 | 381 | CYP2C9RS2253635_GA |

A similar screen of 10,000 SNPs on an Affymetrix DNA chip revealed several other markers associated with adverse muscle reactions to Zocor® (Table 5-AFFYZOCMUSC).

TABLE 5
AFFYZOCMUSC

| | | | | | SNP | | |
|---|---|---|---|---|---|---|---|
| Sample | | | | | consortium | | |
| size | Delta | Marker | Ranked | Chip location | number | Chromosome | base |
| 70 | 0.21627 | 2528 | 1q41 | SNP_A-1510124 | tsc0613997 | 1 | 215,903,182 |
| 70 | 0.213866 | 2517 | 3q27.1 | SNP_A-1509249 | tsc0567070 | 3 | 179,628,143 |
| 70 | 0.171429 | 2523 | 3q25.31 | SNP_A-1513026 | tsc0687812 | 3 | 154,154,665 |
| 68 | 0.162582 | 2526 | 1q22 | SNP_A-1515668 | tsc0047460 | 1 | 152,609,682 |
| 62 | 0.16129 | 2539 | 2p12 | SNP_A-1518723 | tsc0040825 | 2 | 81,733,501 |
| 66 | 0.160873 | 2531 | 9q21.12 | SNP_A-1512095 | tsc1337987 | 9 | 64,218,007 |
| 70 | 0.153668 | 2553 | 6q22.31 | SNP_A-1512342 | tsc1732532 | 6 | 124,421,010 |
| 70 | 0.148413 | 2492 | 4q26 | SNP_A-1509632 | tsc0148687 | 4 | 115,627,058 |
| 68 | 0.139076 | 2511 | 11q14.1 | SNP_A-1513472 | tsc1019659 | 11 | 83,558,478 |
| 70 | 0.128571 | 2520 | 3q25.32 | SNP_A-1507515 | tsc0537907 | 3 | 154,253,501 |
| 56 | 0.125 | 2560 | 5p14.3 | SNP_A-1512670 | tsc0960625 | 5 | 19,861,868 |
| 68 | 0.117201 | 2514 | 21q21.1 | SNP_A-1515706 | tsc0908733 | 21 | 15,438,463 |
| 70 | 0.114286 | 2510 | 5q14.3 | SNP_A-1509310 | tsc0892782 | 5 | 88,403,677 |
| 54 | 0.111111 | 2559 | 4q28.3 | SNP_A-1516638 | tsc0049339 | 4 | 138,455,040 |
| 70 | 0.107143 | 2487 | 11p11.2 | SNP_A-1511438 | tsc0584089 | 11 | 47,664,524 |
| 70 | 0.104633 | 2495 | 2q31.1 | SNP_A-1518568 | tsc0052126 | 2 | 170,563,807 |
| 60 | 0.101515 | 2508 | 10p12.1 | SNP_A-1510527 | tsc0990991 | 10 | 23,715,597 |
| 70 | 0.095238 | 2563 | 9q31.3 | SNP_A-1519460 | tsc0061585 | 9 | 104,492,937 |
| 70 | 0.090734 | 2536 | 13q21.33 | SNP_A-1518231 | tsc0062893 | 13 | 68,267,940 |
| 70 | 0.089683 | 2481 | Xq28 | SNP_A-1517959 | tsc0135188 | X | 145,805,851 |
| 70 | 0.088417 | 2497 | 8p23.2 | SNP_A-1512899 | tsc0630147 | 8 | 5,942,321 |
| 68 | 0.088235 | 2524 | 18q12.2 | SNP_A-1512182 | tsc0492500 | 18 | 37,336,684 |
| 68 | 0.088235 | 2529 | 6q26 | SNP_A-1508303 | tsc0041887 | 6 | 161,240,925 |
| 70 | 0.087302 | 2490 | 11q22.1 | SNP_A-1509996 | tsc0537095 | 11 | 104,305,856 |
| 70 | 0.083482 | 2515 | 6q16.1 | SNP_A-1516226 | tsc0597593 | 6 | 93,798,560 |
| 70 | 0.080952 | 2479 | 15q12 | SNP_A-1514222 | tsc0043313 | 15 | 18,492,700 |
| 70 | 0.076984 | 2568 | 21q22.11 | SNP_A-1512179 | tsc0053101 | 21 | 29,102,747 |
| 70 | 0.076834 | 2504 | 9q31.3 | SNP_A-1508581 | tsc0465012 | 9 | 105,199,194 |

The above identified SNPs were used to make classifications of patients receiving Zocor®. Genotypes were determined from a sample (e.g. blood) from patients suffering muscle adverse effects (case) and from patients not suffering muscle adverse effects (controls). Genotypes were selected for which the prevalence between cases and controls was more extreme than 63%:37%, or 37%:63% (giving a risk of non-response in terms of classification theory of about 3). As shown in Table 6, a Zocor® patient was classified into the non-adverse response (muscle reaction) group with 97% accuracy, and 97% of the cases (individuals exhibiting muscle reactions) were properly classified. Representative portions of several of the genes, including the SNP position, used to obtain this classification performance are listed below (SNPs indicated in parentheses; SEQ ID NOS:28, 32, 38, 41, 43, and 46-90; “Marker” numbers also indicated, below—compare Tables 4 and 5

TABLE 6
0.33 CUTOFF WITH BGA RULES +5 if >9% EAS or >15% NAM
COLUMN BQ NO INCONCLUSIVES

CLASSIFIED INTO

| | CASE | CONTROL | sensitivity |
|---|---|---|---|
| CASE | 35 | 1 | 0.972222 |
| CONTROL | 7 | 28 | 0.8 |
| accuracy | 0.833333 | 0.965517 | |
| efficiency | | | 0.887324 |

2528 ZOCMUSC-RS1416527

(SEQ ID NO:46)

GAGGGTCTTCCTACCCAGGTGGAGTCTGTCTTTGAGCCTGTTAACTCACT
ATCCCAGTTTATAAGCAGCCCTTTTTTATGGTAGGCCCCACGTGGTTGGG
TAACCTAACTGATGTGATAATGAGGACAGACTAAAGGTCACGAGATTGTT
AACA(C/T)GTGTGAGGAGATTGTAGGCTTAGCCTATCAGGTGAGGGAAA
GGTTAGCGTACCAACTGTCTCCACACTTCAAAGCATGTAGCTAGTTTGCT
GCACTAACTTAATTATCTGTGCTATTGATATCTTTGAGGAACATATCTAT
ACCCTGGATACAGTTACTTCACAGTAGGAGATAGCAGGAGGTGGCTTGTT
CGGTTGCCAACTTCCTAAAATGCTTTTTAGAGTCAGCTGGGTTGCTTAGT
CCTCTCTTGCTCTTGGATTAAATATAAAAATAACTATGTAAAGCATATCA
GCCCTCTTTTCCCTGTCTGCTGAA
2517 ZOCMUSC-RS676290

(SEQ ID NO:47)

GAACTGAATTGGGGTGTAGAAGGGTAAGTTGAAAAGGGAGTTTCTATTTT
TTAATTGAGGTGATCCACAGTGTTCTCAGTTTTAAACATTCCTTAGAAAA
CCATTTACAAGTTAAGAGATGCAAAAATGGCAGAGCAACCCAAATCAAAA
CCTGAAATTGAATTACTTTTAGAGGTTTCAAATATAGTCATTAGAGAAAC
TTAACAATACTAATCTTGACCACATTATAGCTCATTTGTGTATTAGTGGA
TAAAATGTTGATTCA(C/T)TATACAATCAAAGGGTTCCATAAATAGAAA
CTGTTTTAGTAAGGTCATTGATACTGATACACTGAGCTCTAGATCCTACA
ATTCATTGAATAGCACCTACCACACAATAGCTTNNNNNNNNNNNNNNNNN
NNNNNNNNNNNNNNNNNNNNNNNNNNNNNNNNNNNNNNNNNNNNNNNNNN
NNNNNNAGAAAATGAGGCTGGGTGACTTGCTCCAGGTCACAGCC
2523 ZOCMUSC-RS1456108

(SEQ ID NO:48)

AAAGAAAAATAGGCACCTTGCATCTAATTATCTAATGAGTAAATTATGTC
ATGTAATGAGTAAATTATGGAAAGTTCAATTGTAGCTGATGTATAGTTCA
ACTGAGTTAAATCTTACCATATTGTTCCTTTTTCTAATGAGCTAAAAGCA
TATACTTTTTTCCTGGCAAAAAGTTCAAATACAATCAGCCT(C/T)TCTC
TTTTTATTTCTATTTTCTTTCTTTTTGGACCCATAATAGGCATCATTCTT
CAAAATGATTTCCATGATTGGGCCAGAAGGCCTGTGGTTACTTTTCTCTT
TCACTCGGAGATTGCACCTCATTTACTAAACAGATATTGTGTAATTAAAG
ATGGGCTGTTTCTTTGAGTTCTCTAGAAATTTTTCAATTCTAAAGTCCCC
AGTGGTCTTTCAAAAAGTTTGTTTAATTGAAGCAAAAGAAATAGTGGGGC
CATTGTTCAGGATTATCAGTGAGAGGTCATTTAC
2526 ZOCMUSC-RS951241

(SEQ ID NO:49)

CTGCTCCACCCTGAAAATGCCGAGTAACAAATGGGACTGGATTTGAACGG
TGTTGTTAAATAGCGCTTCACAAAGCACTCTCCCATGCGCTCNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNCGTCAATCTCTCGCCTGCTGCCCAGATGTGAATCTATCTGTG
CTCCTCCAGCCAAAGGACCGGGATGGTTTCCAGATTCACATGCACATGAG
GAAAAACATCTGGATA(A/G)TTTAGAACTTCTCAGGTCCACAGCAGTGC
AGGTCAGCCTGAAGGGCCGGGGGCTGGGGGAAGCTCATTGTCACCAGAAG
ATGCTTCAGATCTGTTCTCTCCTGTCTGACCCTCCTGGCCCGGGTTGAAC
CATCCTCCCTATGTCAGCCAAACTGGAAGTGCACCCTGGAGGCCCAGCCA
ATGTTTCTCTGGGTACCAAGT
2539 ZOCMUSC-RS717810

(SEQ ID NO:50)

GGGAATCACACATAACCTTAAAGACAACCTCCGGGGACAGTGAGCCAGAA
AACATGCACACTACAGAGTCAGGAAACTTGAATTTATTCCCAATTCAACC
AGAAGAAAGCCAGGTAATTTCAATGAACCACTGAAACTATTTGGATAAGG
GTAAGAAGAAATTAATCTTCAAGAAATAAATAATAGAGGGAAACATGGAG
AAGAACAAATAGAGTGGTAGCGTGTTTTTAAAATGGAGAGCAGTCAATAC
TGCCCTTCTTTTCACAT(A/G)GTTAACCAATCAATCAAATTATCATTTT
TTCTAGATACAGGAAAATGTGAGAATTTTACTATTTTAATAAGTTATTGG
TCTCAGTCTTTTCCTCTCATTCACATCTGTATATTCTGAAAAATCATTTA
TTTGCATTTAGTTGGCTTGTAGAAAGAATGACTCTGTGTCTGTCCCCTCA
CTGTAACCTCATAACCTACCCCTAAGACATAAAATTGAAAAATA
2531 ZOCMUSC-RS2309741

(SEQ ID NO:51)

TGTCATTGACTTGTTATTATAAAAGTTTATGTATGAAAAACATTTCAACC
ATTGGAAGGCTGTGCTCCAAAACCCCCCACAACATCTATAGGGAGGGCTT
TCTTACTATCATCCATGTACTTTGGCAGAATTCTACCTGAACAACCTGTT
AATTTGTTCCAGAGCCCAATCATCTATTTCTACTTATTAATGCATAGTTA
ACACAAATAGCATTTCCAGGCTTCTGCCATGTGATGTGTCAATGTGACCC
CCATAAGTAAACCATAGAGAATCTGAATCTCCAAATGTTTGATCCCTTCT
AATTTGCCACTGCTGCATAGATGTAGCCTTCCCATTCTGTACCTGCA(C/
T)GTGACTTTGAGGCCCTACGACTTAATCTTAAGCTATGACCAGCTTCTA
GAATAAGTTTTCTTCACTTTAACTTTGGCCAGGTGCTTGGCCAAAGTTTC
CCAGCTGCATTAATCCTACAGGTTTGATAATAGGTCTAATCTGTGTCATT
AGTTTCTGAGTCAAAGATCCCACTTGGACAAAATTCGAGCTTTATCAGTA
TAGGCCCTTTACTTTATACTTTGGGGAAGAAGGGGAGCAGCATGGCCTCT
GGCTCCTGGCAATAATTCAACCTTGGTATCAGATCAGGTTTTACATCCCG
CATTGCTCCTTCCAATCTTTCCATGCCATTCCAAGACTCCAAATGCAGTC
TCTCAGCAATGAGGTGCCCAAAATATGAACATCTCTTATTACTGGCTTGT
GGACAAGGGCTAG
2553 ZOCMUSC-RS590944

(SEQ ID NO:52)

CCAAGGGACAAAGAAGTGTCCCGTTATATCTCAAAATCTAGGTTTTGCCC
AGTAACTTAGGACTTCTCATTCTAAGAGACTACGAAGCAAAGAGAGAAGT
CTGCATCCTGGAAGGGCTAAATGATATTGATCATTAGNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNGCTTAAGTC
TATCCCATTAAACTTTTTCTTGCAACTTATGCCCAGGTAAAAGA(A/G)G
ACCAGTAGAATCTTGGAGCAGCTCTTCTATAACTTACTATATAAAGCAGG
AAGTGGACTATAGCGGATACTGTGGTGTGTCACCTAGATTCATCAGTCCT
ACCTCAGGACTGAGCACTCATTCTCCAGCTGCTTGCTTTGCTAGAAATTG
ANNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNA
TAGTTCTCTACCCAAATTTGCTTTTTAGGATCACCCTCAGGTGTTAATCC
TGAGAGTGTGCTCCAGTAAATTTCCTGCATAGAAATCTTCAGAGCAGGCT
TCCTGGAGACCCCTATCTGAGATGAGTCGAGAGAGGAAGGAGATAGACAA
GGAGATGAAGTGCGGGGACCAGAAGAAATATCATATTTTCTCATCATGT
2492 ZOCMUSC-RS4075882

(SEQ ID NO:53)

ATCCATTAGCTTCACAAGTAGCATGTGACCACAGTCCAACTATAACAATG
CAATGAATGATCAGTGACCAATCAGAACTTCTGTGAGAATTTGAAATTTG
GCTTTCAAAGAATGTAGATTCACAAGTCATGCAAATTTTTGTCAGGAATG
AAAGTTCTTTTTGTAGCAAGAAGTAGGCGACTTACTAAGAAAACAGAGTG
AGAAATAATTTGACTCTAGAGGGATGGAAAAAGTACTTGCCCTGATGGCT
TCACAATTCATGACTTCAACAAGGCTCAATT(G/C)TGCTTTTCCCTCAT
ATCCCTTCGAAACATTTCCTCTTTATTTGAATGGATTCCTGTTCCTTGTC
ACCAAAGAAACATCAATACTAATACTCTTGGTGATGCATTTACAAGGAGA
TCTAATGAAGCCCATGTATGCAGGATCATTTTAAAAAGACACTGTAGTTC
TATTTTGTTGGCACATAGATCCAATTTAAGTTCAGTGGCTCAAATTACAC
TGTTTAATTGCTTGAAAATCAGCAGAATTTGGTGGTACTTTCTTAGAGAT
TAACTCTTGGAGTTGAGTAGTATCCTCAAGACTTGTCAAAATATTACCAA
TAAGGTATTGAGATACTTTCATAAGATAACAAAGTGGCTCTAATGATAAT
TTCCCATTCCCAGTTGGCTTACATATTAAATACCAAAAGATAATTCTGAA
CTCGTGCACTTCTATTGCTCACACAGTCTTTTAAACTGAAAGCATGTCCA
ATTAAGGACATTAATGTTTATAAAGTTGAGATACATGTTTTAATACTGAT
TTAATAAAAATTTGTCACATCCTTATTACATGAGATCTCATTGCCACGAG
TAAATCTAGAGGTACATAATGCAGCCCAGTAAATAAGTGCATAGTGAAAT
AATGCGTCTGGCTTAGTACAAGTCAGCTTAGATCTTTTTA
2511 ZOCMUSC-RS1945650

(SEQ ID NO:54)

TCTTGAGATCCATATACCTTTAACAACATCAATAAAGTGGTTATTTGCAA
CTTCAGATATTAATGAAGATAATACTTTTTAAAAATAACTAAACCAGAAG
TTTGAAATATAATCCCACAAAACTNNNNNNNNNNNNNNNNNNNNNNNNTA
ACATATGTTGATCATATTTGTTGGCTCTAGGGATTCCAATATCATTCTCT
GGTTTCTAATATTTAAGAAGCTGGGAATAATTTGTAGCCAGTTTAGC(C/
T)AGTGGAATAATATTTGAGTTAGTTCAACATGACTCCTCCTGTTTATAT
TATTGTTATTGTACTCCATCAGATTTGTAGTTAGTTGCTAAATTCTCATT
GTGAAAAC
2520 ZOCMUSC-RS1378794

(SEQ ID NO:55)

ACGACTTAGGGGTCGAGAAGAATGCAGTCTTAAAAACCTTGCTTTTTAAA
GTGCACTGTTAACTGTTTAAAGAATAAAGGAAGGTTCCAGTGCCCTTTTC
TCCATCGGAGTGTCAGTAGATTAAGCAGGATAAACCTTTTGCCTGAGTGT
TGCTGCAATATTTAAAAGTACAAATCCAATATTTCTGGTCCACGT(A/G)
GATTTCTATGAATAGCTTCCCAGCAAATAACGAAAAAAAGAGAGTACGTA
TGTTGTGAAGTTTGTTCTGTTTATTAGCAACTTCCCGCATACTTCAACTT
TCAACAATTTTACTCTAGAGGCAGAGTAAAGGGATACTTTCTTTAGGAGG
TGACTCTTACTGGACGAACACTCAGTGTTTCCAAAACGTTTGTGAGATCT
2560 ZOCMUSC-RS1911847

(SEQ ID NO:56)

CATATCTGTTGATGTAATTGTTATCCTTGTGTTTTTTAGAAGGAATTGGT
GTGTAGGGGTAAAACAGGAAAGCGAAGAAGATATTAAACTTGCAACAAGA
AAAAGTTAGAAACTTCGATCACAAATGCTGAGTTCCCAAGGTTGTAAATT
TTCAGTTCACTTTGACTTTTCCCAGACTCTTTGGTCAAAACAAACAAGTA
ACAAACTAAACAGTTTTTCTATCACC(A/G)TTGCAAACCTGATATGAAA
TCAGAGAGTAGGTATGTGTTCATGGCAGTTCAAGAATATATCACGAGGTT
AGGTGCATGTGCCTATAAGAATAGAAAGTGGTTCTTCAGAGATGTGTGTA
CTTAGATCAGACATGATATCTATCTAAAAAAAAGAATGATGCTGAGCTAA
GATGGAGGGGAGACCTCCACAGCCCAGA
2514 ZOCMUSC-RS1999288

(SEQ ID NO:57)

TCTAAGAGAGAAACATGCCACGTCAGAAGGAAGGAGAATATGACAGCAGA
TTTTGGAATCAATTGTCAATGTAGTTTGCTAGCTTATCATGGTTTCTTAG
ACACTGTGTGAGCAAATCTCACAAATGGAACTGATTTACCATAAAAATGT
GCCTAATTTCCATTAAATCTTGTTCGTTGCTTTACTGAGGCACTGAAGTT
ACCAATGTT(C/T)CACTGGTTGACCTGCGGGGCTATCTCTAGGTTATGT
TACTCCAGAAAATGAATTGTGTATAAAAGAGGCCTTGGAGGAAGGCGTTT
TATTCGCATCAGTTGTTTTGCACATTGCTTAAGCTAGAGATGTTAGCAGC
TTCTTAAAGAGAGCAGCGTTAGCCAGACCTTTGGGGCAATAGTGGGAAAT
GGAAGTTTCTCTGTCCTATGCTTGATAGAACCATGTCGGTTGAGACACGT
TTCTATCATGCATAGGGCAGGGTATAAGAAATCTGTTTTCAGAGACTTCA
TGGGATAAATGAAGGCTTAAGAGCCCTTCATATCTCAGTTAAAATCCCAG
AGCTGCTATTTGCC
2510 ZOCMUSC-RS256516

(SEQ ID NO:58)

TACACCTGAAGCAAAACTTGGACAAATGTCTTGGGAATGTCTTTCCTGAT
GATTTTCTTTCCCATGCCTCCCTAATCATGGAAGTCAGCCTTTAGTCCAA
AGATAAGGGTATATAAAAAAGCTCTATGGCAGTAATTTGCTAGATGAGAA
CAGAGAATTATTGCTATTTTCTAATTCTACTCTGACCTTGGTATTCTAGA
ACTGAATAACTCTTTCTAAACTTGTACCACATTCCACAAGTTGTTGGATT
TTTCTGACCCTGAATCCTAATATCCCACTAACTTCATTCCTGATACCTAC
ATGTAAACTATCCTCATGTAGCATTTTTAATGCTTAGATCACCATCCATC
ATCTTTCTTTCATTCTTGCTTCTGCTTGCCACATCCTGAAC(C/T)CTGG
TTAGTCCTTGGTTAAGAATACCTTCATCCATTTGGGTCTGTTTGTTTCTT
TGTATTTTAACCCTGGAAAGACTTCCTGGTTTCAGATAGTCTTTCTGGTT
TCATAGTCCATCTGCCCTTCCCCAAAATAACAAAGAGATGTCTTATCTTG
CTTCCTCCTGGTCTTCACCTATCTCTAAAACACCTTCACTCTCTAGATCC
CAGGTTTTCCTAGGCTG
2559 ZOCMUSC-RS1343836

(SEQ ID NO:59)

TCTAGANAACTTCCCATCATTCTCTTATTCCATAGAGGCACAACTNNNNN
NNNNNNNNNNNNNNNNNNNNNNNNNNNNNNNNNNNNNNTAGCTAGGCATA
TGATTGCTTGGTATAGATACTATATTTCCTACTAGACATCCTTGAAGTCA
CTTGTAGATCATACATGAAGTTTGAGGAAATAAAACGTAAGTTAACATGT
CACATTTTTCTTTATTCATTTAAGCATTTATTAAATGTTAGACATGGTAA
TACAATACACATGAGGTATAAAAAGATGAATATTACAACAATTACCTTTT
TATTATTGTCTAGTACACACAATCTCTTATAAANNNNNNNNNNNNNNNNN
NNNNNNNNNNNNNNNNNNNNNCATATGCACTTCATGGAAGCTTTGGGAAG
CCTTTCTTAGAGGATGATTGATGCA(C/T)GCCCCTTGTTCTCTTTCACT
GAAGCTTCAGCCTTCTTGATGGCTGAAACTTCAATATGATGGTGACAACA
AAAATGAAATGGTTGGTACTCTAGCAATGACCTGCAGTGAAGGGAAGCAC
TGTTTCCTACAATGAAGAGTTGGAATAAGCCTGGGACTCTGAGAGCNCCT
GTGAGCCAGAACCTACGTATTAGC
2487 ZOCMUSC-RS1401417

(SEQ ID NO:60)

GGAGCTCTGGGAAAAGCTTGCTGAGCAGGCAGAAGGCTGTGGTCCTGTCC
CACCTGTGAGTCAGTGCCACTCTCTGGGACTGGGGGTTTCCCAGGAGCGT
TGTATTTATGGCCTCCTGCTGCATTCTCCTACCTCAAGGACACAGCAAGT
GCACAAGGCTGCCTACCTCTCTCCCAACTCCACTGTTGCTGTGCTGTTGG
CTTCGCCACACTGCCTGCCCTCCTCAAACTTT(G/C)TGAACCTCAGCTT
GTTTGCTTTCCAGATCAAATCCCTGTTCCTAGTTTTTTCTGCTGCCCATC
AGATAGGTCTCTAGACTAGCTCTGTTCTCTCCCAGGAAGGAACTGAGAGT
CCAGCTTCCCCCTCCTCCCCAAACACAGTGTTGAGCATAACAGATCCTCT
CCCCACAGGAATGGGGAGTGACCCGCTTGACCTTTGAATATGACTCTGAA
CCCTTTGGGAAAGAACGGGATGCAGCCATCATGAAGATGGCCAA
2495 ZOCMUSC-RS951917

(SEQ ID NO:61)

CTGGGAGAACCCATAGTCTTCCACCATCACACTGTAACACTGATTAGTTA
ACCATATTAGTTAGTTGAAGCCTTAGATCTGGAGATGATGAAAGTTAGAG
CCAGCTAAACTGGAGGAGTGGTGTGGCAGGGAAAGAAGGAAGCCCAGGAG
GTTGGACACAGTAATCCTAGGCTGGAGAAGA(C/T)ACAGGGAGGATGAG
AAAACAGAAATTTGGGTGCCTAGAAATCTGGCCCTAGAAAAGGGAAGGAA
TAGTTTTGTCCAAGATCAGTCCTAGCCTGAAAAAAGAAGAAAAGGGAGAA
GTAAAGAAAAGGGAGAACAGACCATCATCAAGTCAACTCCATTAACTTTG
TGTGTGTTTCCATTCATGAAGTTTTGTTATAAACTAGTGATAGGAAACTT
TAAAAATCTAATATTACCTAAGGTGGGATTACATGATTCCCTTATAGGAG
GCTGATATGTGGTAGGATTTGGGGATTTTGGAGATGGTCCCAAATCTTAG
CTAAAAGTATGTCCACAGTCCCCATTAGGTGTCTGAAATTCTGTTAGGGA
AGGTCTTCATGACTTGTCTTCCCTCTGCCAAGGAAAATGGC
2508 ZOCMUSC-RS1929609

(SEQ ID NO:62)

AACAAGTGTTTAAAATGTTTTGATCATGTTTTCTTTATGATGAGCTATTT
TAGCCCTCTTCTAAATGTCACTCTAGAAACATACCTACATAATTGAATAG
ATTTATTAAAATCATTTTGGCAATATTTTATAAGTGGTGNNNNNNNNNNN
NNNNNNNNNNNNNNNNNNNNNNNNNNNNNNNNNNNNNNNNNNNNNNNNNN
NNNNNNNNNNNNNNNNNGGTATGCTATAAATATGTACAAGAT(C/T)GGT
GCTAACTGGTTTTCAGGGGCCTATAGGAAGGTAAGTCCTATGAGAAATAA
TTAATTTTTATTCTTATTCTTGTCTTTTTAAAATTTGCTTTTGTGTTACA
GAAAATGAGAAGCTGCCTCCAGGATAGGGGGAGCTTGTTTTGCTGTGGAG
ATAGCTTCTAGAAGGACAGAATTATGATTCTCCATAGTTTTGCCAACATG
TATTTGCTAGAGGGTTTGGCTTAGAAGCTTTAATGGCTATGGAG
2563 ZOCMUSC-RS1986710

(SEQ ID NO:63)

AGTGCCAGGACCTATTCTAGGTGCTGGGAAGTAAGATAAAGCCCTGGCAT
CATAGAACAGCCATGATGATTCTTCACAGAGATTCATGAGGTAGCAGTGA
GAGATTAGGAGTTATTAATAAATAGCATTTTCTTTGTAAGTAAATGACGA
TGAATCCCAGACTAAAGGATTCACAGGTGGTCAAACTGTGAAGCTAGGAC
CGGCTAGAGAACAGCAATCAGGTCTCATGA(A/C)CCCAGCCATTCGTCA
TTTCTTCCATACATGCCAGACATGGCCCTTCACTGCTGATCAAAAGGACA
CAGAATAACAGAATGGTGCCTTAGAT
2536 ZOCMUSC-RS2018205

(SEQ ID NO:64)

TCTAGATCCAGAACTTAGCTTGGTTCTTTGTTTTCTAATTAACGAAGAGC
TCTTTCATCTGATGCCATGAGAAAGC(C/T)TGTCAATCCTGCTGTCAGA
TTACTCCTATATCTGCCTTTCCATGGGAAATATTTTAACGTGCTTTACAG
GACAGTGAGTGGGATTTCACTAAGCTACAACAATACATTGGTGACTGCTT
TTCTTTCTAGTTGCTAAGAAATGATGGTGTCTATTGCAAACTCCTCTTGT
CACTTGCTACTATTATAACACACAGCTGGTAAATGAGGTTCTGAAGCCTG
ATAGGGTCTACCTACCTTTATTTATTAAAACATACCCAAATTTATGAAAA
GAATCTGACAATTGTAAATATGTGAATTAATTTAGATCAATTCATCATGT
ATCCAAACTTGGCAGAATTCACCAACCCAAAACTGTCAGTTCAACCTTAA
TCCCTCCGTGTTTCCTCTGCTCCAACTCTACGCGTTTTATAATCCTGCAA
A
2481 ZOCMUSC-RS750841

(SEQ ID NO:65)

TGCTTTTCTGTTGAGGACTTTAGTGGAGGAGTGTACACAACATATGCTGA
TAAAATAACACCAGAAGGAACTACCAAAACATTTAAGAGATCACAATCAC
CACCCTAACCACTGTGGGTTTTTGTTAACTCCACTATTGTTTTACTAGAA
TCTAGACTCTGACAAAGGTAGGATTGGACAAAGTTCACTGCCTGTTTCCC
AAAATTCTCAGATGTTTCATTCTTAATGAAGTCTCAAAATT(A/T)ATGT
TCCAGTCCCAGGCCACATATAGTTGTTGGACAGTAGACACCATTCAGAGG
ATTCTACTCCATTCACTGGCCCTACAAAAGCACTGATTCTTGTGCCATCA
CTCTCAGCATTCCACTTTCTTCATCTTTATTCCATTCCTCAGTTTGACAT
TTAATACTGAAATTGTAAAGTACCAAAACATATTTCAATTGTCAATTCAA
ATGATTTCATTTGCCTTTCACTCGTTCTAGATTTTCCCTTCCTCCAAACT
CTCCTTTTGCTTTTTCCCTACACTTATTATTGGTTACTTATACTGATACT
TACCCAGAATGTAATGGGAAAAGCACTGCATCTGATTTTATAACATCTCA
GTATAAGCTCTAGCAGNNNNNNNNNNNNNNNNNNNNNNNNNNNNNNNNNN
NNNNNNNNNNNNNNNNNNNNNNNNNNNNNNNNNNNNNNNNNNNNGTTTCA
GCACAGAGATATTTAAGGTCCATTTCAAAAAATATTCTATAATAGGAGGA
AAAGGGTAGATATAGCCAGCCTCATAGAAAGAAATGTCAGTAACAAGTGT
GAAAGAAAAAAACATT
2497 ZOCMUSC-RS1424724

(SEQ ID NO:66)

CTTACTATTGCACTAAACTTAGAATGCATAGTGCCCTCACCACCCCCTGC
AATCTTTTCTTTCACTCATTTGATGGGGGAAAATTCAGTACATTAGATGT
TTCCAAATATACCCCTATTATCAACAGTGGCCCTTTATAAATTTCAATNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNTCAACAGTGGTCCTTGACCAGCACTCTGCTGCCACCCAA
AAAATGCATGAGAAATGG(C/T)CTCTCCGTGAGCTAGTTATCTTCTTTT
GCCATCCGGTTCTATTTTTATCAGTTCATTACACTCCTTCTGACATTGGC
TAGGATCCAGGCCCTTAGAAACTGCCTGGCAAAAAGCACTCTAGAGAGGG
CATGCTGCTACATAAAATGCATCTGTCACCCCAATTTATGCAAATCACAG
GCAAGGACCNNNNNNNNNNNNNNNNNNNNNNCTAGTAAGAACTTTCTAAT
ACAGAAGAAAGAAAATT
2524 ZOCMUSC-RS1355828

(SEQ ID NO:67)

TCTCTAGAAACAGCGGAAGGAGAAAATGTCTCTATGAGATTGAGTACAAC
AGCAACTAAAGGGACTGAGGTCATGCATTCCACTTCTGAGCTTATGGCCA
CGTCCAACTCTTACAAAAAATAACCTTTCTCTCCTGCACTCTTAGTAGTT
TGGCCAATTTTATATGGACTTGTGCTATTCCTTTACTTTTTGGCTTAAGC
(A/G)TATATATGACTCCCAAGGCCATGAAAGCCCTAACCATTTGGCTGA
GGGTGGTATTTTCAGGAACTCCACCAGTGAAGCAGATACTATGTATTGGA
CAACATCATGTGTTCCTAATGATAGGTTCTTATTCAAATGACAATAAATA
TTGTTAGAAAATTCAGGCGTAAGTTTGAGTGACAATAGTGATAAAAGGAA
AGTCT
2529 ZOCMUSC-RS718319

(SEQ ID NO:68)

CAGTCAACTAGTTTCTATTTTTGCACTCATCTGCTCTGATCTGTTTAGGC
AGAAGCTTAAACAATTCTGCTTGCTCAACTAACTCAGACACTGTGGATGT
TAGGATGAAGGTACAGTTTGCATATTAAAAGATA(A/G)GCCAGTACATC
TCTGAGATTTGCCATCCTCTCTGGTAAGGACTGCACTCAAAGTAACAGGA
GGGCCTAACCTTAAATAAATATGCAGCCATACTGCTAGGGNNNNNNNNNN
NNNNNNNNNNNNNNNNNNNNNNNNNNNNNNNNNNACAGGCTTTCTGACAA
GTGATTGATAGATTTATTTCAAAGCTGTCAAGTAGAGCCCAAAAAGAGTG
GAATCTCACGGTTCTCTTGGCAAGACTATGGTTCTCAGATGGGCATACAA
GCTCTTTCCTTTCTGGGACAGTCACA
2490 ZOCMUSC-RS1378335

(SEQ ID NO:69)

GTTCACCATCTAAATCAGTTGTCCTTTATGGTAACACAGGATTCACAGAG
CAGCAATCTCTTTTTGGGTCAGGCAGCAACCTTTATTTGTGCTCTGCACT
TCACCCACATGTTTTTGTCTGGGATATTTCCCCGATCCCATTCTTCTGTT
GTTTCACACTACTT(G/C)TTGTCCTTCTCTCCCACACCTAGGCAGTTCC
AAGCCTGAGCCTTTTCTNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNTGCTG
GTACTAGTGACTCAGCAACACTCCATGACACTGAGCTCCTCTGCCCATTG
CACCTCCTCCTGCAGG
2515 ZOCMUSC-RS1408282

(SEQ ID NO:70)

TGATACACTTTTCATTAATGTTTATTCTTGTATTTCTGCTGAAAATATTT
CTGTTAAACTAGTTAAATCTTCTATATTTCCATTCAGCAGAACATATAAT
AATGCTAGATGATTGCTGTGTCAAAAAATATTTTGTTCTCTCACCTTATA
CTAATGACGGTAATAAATCTGACCCTCCAGGATTTTCTTATTGCTTTTTT
TTGCAGGCCTT(G/A)AGTATGGCAGAAAACATGATGTAGTGAAAAATAC
CTGGGGTTAGTCTAGACCCTGTCCTGTTACGATCCAGCTCTGACTCCTTA
AGTCCATCTACTGACATCTCAGAAAATGTTTGCTCATAAAGGAAAAATAA
TATTTCACATGGATGGATCTGTATTTCACAAGATAACATAACGTAGTTGA
AAATGAGTAATAAGGCATAAAACTCTACAATATTATAATTGCAGCTCCAC
AGCTTTAGGAAAATTACTCCCTGCTCTGTATTCTCTTATCCATG
2479 ZOCMUSC-RS718978

(SEQ ID NO:71)

TCTAGAGAAACCTCCCAAAAATAAGGCATACCATCCCTCTGATGGTTNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNAGATTGAGGAAAGGAAGCAGGCGGGCAGCTGGT
GGTCTTCCAAGCACTATAGCTGATCAGCAACCAGAGATTCCAGAGAAACA
TGGAATGATACCTTAAAGTGCTGAGGCTAACTAACTGCTGAGCACAAAGG
ATATTTTTTCAGGCACACACAGGGATTCTGAAAGTGTAAAGACGAGTCCC
AGGCCCTCATTTGAATATTAAAAAAAAAACTA(A/T)AGCTTGATCTTCA
GGAAAAATGGAAACGAATTCAAAAGCAGAGACTGGGATACAAAAAACAAT
GAAGAAAGAAGTCCGTGAACATGTTGACAAATCCAAATTAGTATTTCTTA
TAAATAATAACATTGATAACTGATCTAGA
2568 ZOCMUSC-RS723470

(SEQ ID NO:72)

TCTAGATCCTTTTATAGACATTAACATATGCTTTATACTTTAAGTTCTAG
GGTATATAAAAAATAAAACACATAAGCTAATGAGTTCTCTTTTCAATTCT
TAGGATTTAAACTGTCTTATGGAGAGATACCTAAAGCCTCTTCAAAAAGA
AACTTTTCTCACCCAGGATGAGGTATGCTGGAAATGGTTTCAACTTTGTG
ACCATCATACCGTGGTTTGACTTGTTGGTAAAGAGGAAAAATGGGGACAT
GCG(G/T)AACACTGAAAACATTTTAAATTTATTTAATTGTAACTAGCTG
AAATTATTGCCTGCTAAGCTTTGGTGTGCTCATGAAGAGTAAAAAACTGC
CCCGCTCTCTGGTAGTGGGATTATTCTCTTAATTGATAAGAATTGATAGA
AAAACCTTGTAATCAGATATAAATAACGTTTAGGAAGACTAATGAGATAT
GAGGTGCATTAAATTCTGTTTTATTTCTAGCTTGACGTGCTTTTTGGAAA
TTTAACGGAAATGGTAGAGTTTCAAGTAGAATTCCTTAAAACTCTAGA
2504 ZOCMUSC-RS1570303

(SEQ ID NO:73)

AGAAATTGAAAGGATAGCTAGTAATTTTTTTTCTTCCTAGTTCAAAATCT
TCCACAAGTTTTCCTTGCATCTTCTCCAAACTTGCTGTTTTTTTTCCTGT
ATTCATTATCTTAGTGAGTAACATTGTCTACGTAGTAACTCTAATTAAAA
ACTTTGGTATCATGTTCAACATCTCCTTTGATTCTTGTGTACCCCTTTAC
TTACCAAGTCCTATTCTTTTTCTTAGCT(A/G)CTAAAATCTGTTTCTCC
CTCATTCGACTTTTTGCAATACTGCCAGAATTGTCTTTCAGAAATATATA
CTTCTACATAATGCTATTGTTCTCATACTTGGAAACATTTTTGTTTTTGA
ATAGCACAGTTGATAACATCCTAACTCTTTGGTGCAGCATACAAGATCCT
CAACAGTCCTTATCCAAACTTGCCTTCCTAGCTATATTTACTACTCGTTT
CTTCCTAAATTTCCCCAATGCTCAAGCTTCCCTGAATCCATGATATTTTT
TGATACTTTTATACNNNNNNNNNNNNNNNNNNNNNNNNNNNNNNNNAAGT
TTCATTATACCTTACTTGTTTCCGAGATCCAGCCCCCATTTCCCTTCTGT
TGTGAATCCCATTGCTAGTGTTTTTAGACAGTCAGCCCCTTGTTCCTTAG
CACACTCAGTTGTGCTTTTCTTGCTTGCTAGATTCTTCTTCTAGATGGAG
GGTTCTTGAGGCCACGGACCTGTCTTGTCTTTTATTCTCTTTTTGTGCTC
AATACAAGGCCTAATGCCCTGCATCATCATTTTTGAAATGAAAACAACTA
AAGTTTTTAAAAGCTCAAGTGACTAAGTAGATGCTGATAGTATTAACTGA
TACACAGAGGGATAGATTGGTTTTTGAGCATGTTGAGTTTGAGATATTTA
TGTTGCATTTAGAAAATGTTCAGGACATAGTTGAGAATAGGAGGAGGCAA
GAAAAACAGTTACAAGCATTACTAGTATGTAGGTAATATTCCAAATCATG
GAAGTTTTTGAGACTATGTGCAGAATGAGAAGAGAAGATAGAGAAGAAGA
TAAGTTGCACTAGTGACTAGTGGGCAGGAAACAAGAAAAAAATGAAATGG
GTAGCAGTTAATTTTCTCCCTCTTTTATAGGATATAATGG
2663 GSTM1PE1_287

(SEQ ID NO:74)

GAAGCAGCTGCGGGACGGACTCTAGGGACCGTTCCTCTTCAGGGCTGCCC
GCCTCAGAAGGGCCTGTGCATGCCGCTGTGTGTGTGTTGGGGGTGTGGGC
GGGTAGAGGAGGCAACGGGTACGTGCAGTGTAAACTGGGGGCTTCCCTGG
TGCAGACAAAGTCAGGGACCCTCCATCTCTGAC(G/A)CGACCTGCGGGC
CATCTCTCCCAGCTGGCCCACGCCATCCGCCTGCTCCTGGAATACACAGA
CTCAAGCTATGAGGAAAAGAAGTACACGATGGGGGACGGTAATGGCACCC
TCGTGTTCGGGCTCTGCCCACTCACGCTAAGTTGGCACCAAGCAACCCAT
GGTGGCCACCTGTGGCTGCCTCTGCAGGCC
324 CYP2B6RS2279343

(SEQ ID NO:75)

GTTCAGAGGCAGAGGGGAGTGGGGAAGTGGGGTTCCCATGGAGGGATTGG
GGCCCAGGAGGCGCTCTCTCCCTGTGACCTGCTAGCTCAGCCCTAGGCAA
ACCTCACCACCCCTTCTTTCTTGCAGCTGTTTGAGCTCTTCTCTGGCTTC
TTGAAATACTTTCCTGGGGCACACAGGCAAGTTTACAAAAACCTGCAGGA
AATCAATGCTTACATTGGCCACAGTGTGGAGAAGCACCGTGAAACCCTGG
ACCCCAGCGCCCCCA(A/G)GGACCTCATCGACACCTACCTGCTCCACAT
GGAAAAAGTGGGGTCT
2672 CYP2D6_RS2004511

(SEQ ID NO:76)

GACTCTGTCAACTAGTCCTGCGCCCGAGAAGCTCCACAGTACCCTCTCCG
ACCCCACAGCAGGGCGCAGTCACACCTCTCAGAGGCACCCACACTGCCCC
CTCTCCCTGCAGGCG(C/T)TGGGTCCTCCAACATTCTGGCAGGTCCTGG
TTTGTCTCCCCACTAGACGGGGGCTCTGGATGGACAGGCCAGCCCTGCCT
ATACTCTGGACCCCCCACCCAAGTGGGGACAGTCAGTGTGGTGGCATTGA
GGACTAGGTGGCCAGGGTTCCTAGAGTGGGCCCACCTGGCAGTAGCCATG
CTGGGGCTATCACCAGGGGCTGGTGCTGAGCTGGGGTGAGGAGGGCGCCA
GGCCTACCTTAGGGATGCGGAAGCCCTGTACTTCGATGTCACGGGATGTC
ATATGGGTCACACCCAGGGGGACGATGTCCCCAAAGCGCTGCACCTCATG
AATCACGGCAGTGGTGTAGGGCATGTGAGCCTGGTCACCCATCTCTGGTC
GCCGCACCTGCCCTATCACGTCGTCGATCTCCTGTTGGACACGGCCTGGA
CAGACATGCGTCCCCACAATGGGTCAGCACCCAGGGGGTCCGGCCCTGAC
ACTCCTTCTTGCCTCCTATGTTG
65 CYP1A1_RS2606345

(SEQ ID NO:77)

GGGCTAAATATCAAAGCACACTAGATCCCTGAGTCAACAGGGGACAGTGC
CAGGCTTTGGTGTGCTATGGTGTCCCCTTCTGCACAGTGGGTATCCCAAG
GACCTGCAGCCTTTGTAAGATGGTAGGAGGCCAAGGGAGATGTGACTGGT
GAGTTTTTAGGGACTGGTATTTCCAGCTCCCTGCAGTTGGCAATCTGTCA
(A/C)CCTGATTGTCTCCCAGCAAAGGACAAAGAAGATGGTCAATGAGAT
AGGAAAGGCCTGGGACTCCTGCACAGATCTGCCTCTATTGTCCCAGCTGT
CTAGGACATCTCAGCACTGACAAGCCACCCTTAGGAGGGTAAAATCTGCC
AGGGGTAGCTTCTTAGGCTAGGGCAGGGTACAGCTGGTAAGAACCCAGCC
CACCC
225 CYP2C8_RS1891073

(SEQ ID NO:41)

TGAGATATGGACTCCTGCTCCTGCTGAAGCACCCAGAGGTCACAGGTAGG
ACCACAGATGATGAACAAAGTGAATTTCAGAACAATGCTGAGAAGATGGT
GCCAGTATCCTCCACCTTGTTTCTCTCAGAGAAGGCTCATTCTTTAAATT
TCTGTGTCATCAGCTGTAATCTGTCTAAATTTGATGACACAATTTAAAAT
GACATCTTTGTACAATGGAGGAGGATGACAGAGATCAGTAGAAACAGTAT
GGCAGTAGCAAAATAAGTAAAGCACTGATGAAGTGTCTGGATTTCAGCAA
AGGTAATTTGTGGTAAGGAGAGCCAGCATAAATTGCCCTAGTATTGAATG
TTGGTTTTATTATGAAAAGTCCACTTTGAACAGTAGGTTCATTTCTCATT
TTAAAAATTCCATGCTCTAATGCTGTGGTGGGGAGATGAAAACAATCTTT
ATTGAAGCATAAGTGGAAATTCTAGAATTGTACTGAGGCATCCTGACATA
AATTCCAGTCTGGGAAGTAATCTAAAAGTTAGTCTCTTACAAAGGTGTTT
CTATTTAAGCAGAGGCCATACCTAAAGGAATTTTATTATTCTAGGAGTGT
GTTTCATAAAAATGCTATTTGACCAAATAGGGACATTTGGAAGGGGGTTT
AATAATTGCATCTTTCACATACAACTTTTCTTTAGAATTTACAATTTACC
(C/T)TTAGAGTAAACTCTACATTTCCTTAGAATTTACATTTAAATAGTT
CCTGCTTTGCAGCAGATAACATACCTTTTTCCCTGTGTCAGGATCCCACT
GAAATTTCAATAACTAAATTACAAATTAAAATTATCCTGCTGAAGTGAAA
AGAGAGGAGAATAGAAAATAGCCAATAAGTCTTTGGCAAATTTCTAAAAC
ATGGAGAGACAATGGAGGGCTGACAATTGACACAGCATAGAGGATGGAGT
TGTAAGTATTAGCAGAGGAAGATTTATCAAGAGGAGAAGCTAATCTGTGT
CACTGTGATTCTCATAAGGTTCAGTATTTGGAGGCACCAAGAGCCAAAGA
CAGTAGGGAATGAGACTGAAAAGGGAGGCTCTCCTGCCCAAGTTTCCTCC
ATAACTCCTTTTATCAGGTACCTGATTTTTTCCCACTGGGAAAGGGAAGC
ATACTCTCCAGAGAGACTGAGCCTAAGAACCCCTGTACTCGGCGACACTT
TACAGGGCCAGGAAACTGTGAGATGTGAAGACAGAAAAAAAAAAGGTTTG
AGAAAGATCTATTCCTCAGATTCAAAGACTCAGCATTTTTCTTCATCTTG
CTTCCTGACATTCAGCAGCCAGCTAGATACTCTCCAGAGAAGAAACAAGT
CTGGAGAAAAGACCTCCGTATTTTGCCAACTTAATAGAAAGTTATCTATA
249 CYP2C8_RS1891072

(SEQ ID NO:78)

TAATCTGTCTAAATTTGATGACACAATTTAAAATGACATCTTTGTACAAT
GGAGGAGGATGACAGAGATCAGTAGAAACAGTATGGCAGTAGCAAAATAA
GTAAAGCACTGATGAAGTGTCTGGATTTCAGCAAAGGTAATTTGTGGTAA
GGAGAGCCAGCATAAATTGCCCTAGTATTGAATGTTGGTTTTATTATGAA
AAGTCCACTTTGAACAGTAGGTTCATTTCTCATTTTAAAAATTCCATGCT
CTAATGCTGTGGTGGGGAGATGAAAACAATCTTTATTGAAGCATAAGTGG
AAATTCTAGAATTGTACTGAGGCATCCTGACATAAATTCCAGTCTGGGAA
GTAATCTAAAAGTTAGTCTCTTACAAAGGTGTTTCTATTTAAGCAGAGGC
CATACCTAAAGGAATTTTATTATTCTAGGAGTGTGTTTCATAAAAATGCT
ATTTGACCAAATAGGGA(C/T)ATTTGGAAGGGGGTTTAATAATTGCATC
TTTCACATACAACTTTTCTTTAGAATTTACAATTTACCCTTAGAGTAAAC
TCTACATTTCCTTAGAATTTACATTTAAATAGTTCCTGCTTTGCAGCAGA
TAACATACCTTTTTCCCTGTGTCAGGATCCCACTG
74 CYP2E1RS915907

(SEQ ID NO:79)

CGTGCAGGACTTCCAGGCCTCCCCAGCTTCATCCACCTGCAGGTGCTCAG
GATCCTGATCCCCTGCCCCCTTCCCACCTTGGTGAAACTTCTTGTATCCT
TGTCTTGTCCTTTCCTATGGCTTGTGGCTCAAGAACAAATGTGGAGCCCA
CACTGATTTCCCAGGACTGTCTGAGCATCTTCTCCACCAGTTTGGCCCCT
CGTGGCAGCAGA(A/C)ACTAGCCCTGTAGCAGGAGGGGTTAGCAGGAGC
CGTTTAGCTCCTGCCTGAGCTATGACCAAGGTCAGGGGGATCTCACCTCT
CCCAGGATGGCCCTCATGCTGTGGAGGGAGACAGAGCCCTGGCCTGCCCT
CAGCAGATTTCTGGGAGCCTCAGTTTCCCTGGCTGTGAGTGGAGATGACT
CTGTCTGTCACAGCTCCAAGTCACAGTTCCACTGGGAGAGCCTCTTGGAC
ACTGTCTCCTGTGTCCCTGTGGAGCTGGGAGGTGGCTGGTTCTGTGCTGA
AAGGAGACAAGCAGCCCCTTCTCTCCGGTCTGTCTCCGGTATCACAGGAA
AAGCACAGTGCAGAGCGCTTGTACACAATGGACGGTATCACCGT
134 CYP2C8E2E3_397

(SEQ ID NO:80)

AACTCCTCCACAAGGCAGTGAGCTTCCTCTTGAACACGGTCCTCAATGCT
CCTCTTCCCCATCCCAAAATTCCGCAAGGTTGTGAGGGAGAAACGCCGGA
TCTCCTTCCATCTCTTTCCATTGCTGGAAATGATTCCTAATAAAAAAAGG
GGCAGAAACTGGGAGAATTCACAGCCAAGGAAGAAAGTGCTGCAACACT
(C/T)GGCAGCCATGCAGATAGGCTAAGCTCTGCTGAGAAGCTTTTTAGG
GCTCTGTTTTCCATCCCCCTCACCCCAGTTACCAAAGCTGACACAGAAAT
ATGTGCACCTACCAAGTCCTTTAGTAATTCTTTGAGATATTGGGGAATTG
CCTCTTCCAGAAAACTCCTCTCCATTATCAATCAGGGCTTCCTTCACTGC
CTCAT
373 CYP2B6RS2014141

(SEQ ID NO:81)

CTTACCATCACCATTCAGGATGCTTGCAAACCAACTGCTAGCTGCACCTG
TAAACACATATCTGTTTCTTTCCCCTGAGAAATGTCCTTAGTGTATTTGT
GGCAGTGTCTAGAGAAACAGCAGTGTCAGCCGCATTTTAAATTAAGTTAT
CTGCACTAGTGAATTCACTGGAAAGATAAGAGCAATATTTGGTTTTCTTC
AGCACCATACACAAGCCTCCAAGATGGGCATAGAGGAGATCTAAAATTGC
GTGATGTTCCATTAAGCGTTTTTGTTGCCACAAATGTTCTCATCTCAGTT
TGGAGAGTGGCTTCTACCCATCTGAACTCCTTGGAGGTTCAATTAGCTGC
AAAATTCAAGATGTCCCTTAATGTATAACTTAGCCTCAGATTCCATACAA
CTGTCACCCAAATACCACCAAGA(A/G)TGAGCACCCAGGAACCCAACTG
GAACCTTTTCTGAACAGAAACCAACTTATCTTCGTCGATTTTGAGGTTGA
TAGTAATTTCAGTTATTGACTGTTTTGGCTTTTAACTATGGGAGGTATTA
GGAAACTCTCAGGGAAACAATTTGGAAAGCAGCAGTGAGCTAGGCCAAAT
AGCAAGTTCTGGACCTGTGAGGAGAAAGAACAGAGTAAGCAAACCTCAAG
ATACTCAAGGTAGGCACTCGTGGTGTTGGAAAAGAGGGTCACCTACTGGC
ATTAGAGCAGAGATCAGTTAGATTTGTTTACCCATAAGTCTGCATAGCTC
CTGAACAAGGTGGGAAACTTACTTTTTTGTGGTCTTTTTCTAGCATGCTG
CGAAGGTGCATAACCACATTTAGTTGGAAAGAGACTTTACTGTATTTA
172 CYP2D6_RS2267446

(SEQ ID NO:38)

TGTGCGGGAGGACGGGGACAGCATTCAGCACCTACACCAGACAGAACGGG
GTCTCAATCC(C/T)TCCTGTGCTCTGCGTTCATCTGGACCAGTCTCAGG
CCCCAGCCATCTCCAGGAAGACCCA
179 CYP2D6_RS1058174

(SEQ ID NO:28)

GTCATCGGTGCTGAAGGATGAGGCCGTCTGGGAGAAGCCCTTCCGCTTCC
ACCCCGAACACTTCCTGGATGCCCAGGGCCACTTTGTGAAGCCGGAGGCC
TTCCTGCCTTTCTCAGCAGGTGCCTGTGGGGAGCCCGGCTCCCTGTCCCC
TTCCGTGGAGTCTTGCAGGGGTATCACCCAGGAGCCAGGCTCACTGACGC
CCCTCCCCTCCCCACAGGCCGCCGTGCATGCCTCGGGGAGCCCCTGGCCC
GCATGGAGCTCTTCCTCTTCTTCACCTCCCTGCTGCAGCACTTCAGCTTC
TCGGTGCCCACTGGACAGCCCCGGCCCAGCCACCATGGTGTCTTTGCTTT
CCTGGTGAGCCCATCCCCCTATGAGCTTTGTGCTGTGCCCCGCTAGAATG
GGGTACCTAGTCCCCAGCCTG(C/T)TCCCTAGCCAGAGGCTCTAATGTA
CAATAAAGCAATGTGGTAGTTCCAACTCGGGTCCCCTGCTCACGCCCTCG
TTGGGATCATCCTCCTCAGGGCAACCCCACCCCTGCCTCATTCCTGCTTA
CCCCACCGCCTGGCCGCATTTGAGACAGGGGTACGTTGAGGCTGAGCAGA
TGTCAGTTACCCTTGCCCATAATCCCATGTCCCCCACTGACCCAACTCTG
ACTGCCCAGATTGGTGACAAGGACTACATTGTCCTGGCATGTGGGGAAGG
GGCCAGAATGGGCTGACTAGAGGTGTCAGTCAGCCCTGGATGTGGTGGAG
AGGGCAGGACTCAGCCTGGAGGCCCATATTTCAGGCCTAACTCAGCCCAC
CCCACATCAGGGACAGCAGTCCTGCCAGCACCATCACAACAGTCACCTCC
CTTCATATATGACACCCCAAAACGGAAGACAAATCATGGCGTCAGGGAGC
TATATGCCAGGGCTACCTACCTCCCAGGGCTCAGTCGGCAGGTGCCAGAA
CGTTCCCTGGGAAGGCCCCATGGAAGCCCAGGACTGAGCCACCACCCTCA
GCCTCGTCACCTCACCACAGGACTGGCTACCTCTCTGGGCCCTC
2739 CYP2B6RS1038376

(SEQ ID NO:82)

TGGTGCAATTTTTGTTCACTGCAACCTCTGCCTTCCAAGATCAAGAGATT
CTCCAGTCTCAGCTCCCAAGTAGCTGGGATTACAGGCATGTACTACCATG
CCTGGCTAATTTTCTTGTAGTTTTAGTAGGGACATGTTGGCCAGGCTGGT
GGTGAGCTCCTGGCCTCAGGTGATCCACCCACCTCAGTGTTCC(A/T)AA
GTGCTGATATTACAGGCATAATATGTGATCTTTTGTGTCTGGTTGCTTTC
ATGTTGAATGCTATTTTTGAGGTTCATGCCTGTTGTAGNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNGAAGGAATGTTGGTGAGAATGCCTCTCTCACCTCAATGCCAACTCT
GTGAAGGGCCAAACCATTGTCTTGCTCATCCCTGTACTCTCAACACAGCG
TGTGGCATATGACAGGTGTTCAAAATATTTGGTGAGGAATGAATGAATGA
GTGGCTAAATCAGCCACCCCCTACCCCCACAGCCCA
98 CYP1A2E7_405

(SEQ ID NO:42)

CTAGAGTATACCAGTCCACTCCAGGGAAGATTGGAGCTGAGGCTGCTTGA
GGGCTATACACACTCTGGGAACTAGGGGGTCTCCAAACCCTTGAGAGGTT
TGCAGGAGGAAAACTGCAAGGAGACTGGCAGAAAGCAGGCTGAAGTGGAA
GCTTCCTGGCCCGTGCTGGGCTC(G/C)TCAGTGCTTGAGAACATAGATG
AAGGGCAGACAGTGGCCGCAGACGAGGGACGCTGTGAGGAGGAGGCCTGG
CATGTCTTGGGGCCAGGAAGAGCTCCCTGATCATTTTTTCCTTCAGGATG
GGTAGGCTCTTGGTCTGACTGCCCGGAAAACAGGTGATGGAAGGACCAAG
GACTACAAAGGTCAGGAC
108 CYP3A7RS2037499

(SEQ ID NO:83)

ACATCTTAATTTTTGCTCAGCTCAAAACAGTTTCTATTTGCCCTTTTAAT
TTCTACTTCAACTTATTGGTTGCTTAAAAGTTTATTGTTTAACTTCCACG
TATGTGTGAGTTTCCCAATTTTCGTTACCTTATTGATTTTAGCTTTATCC
TATGATAGGTGACAGAGATACACTGTGTTATTTCTATCTGATTTCTTGAA
(G/C)AGCATGATTAATTTAAGCAGCAAATTATGATTTGGGTTATCCTAA
GAGACCCTAGCCCAATACATAAAGGCAAAGAGATTAGGGCATTGAATTTT
GTTCCTTTCATCATTCAAAAGATGCAGAAGGGGCTGCTGATCTCACTGCT
GTAGTGGTGGTCCCTATGCACAGACCTTCCCTTGCTCAGCCACTGGCCTG
GAAGA
2665 3A4E10-5_292_712037

(SEQ ID NO:84)

GAAGTGGTCTGTAGACCAATATTGGTCCCTTAAGTTCCCTCAAACATATC
TTCGGGAAACGTCCTTTGATTTTCCCTACATTTAACCATTAGTGTTGCAA
ATTCTCTCAAAGTTTGTCAAGATATATTGTAGCTAAAATAAATTACATTT
TTCTTGGGGGAGAGTACTACCTCATATTAACTTACAATAAAGTACTTTTA
GGATCATTCAAGGAACACACCCATAACACTGAGTATGTTATGCGGAAATG
CTCTCTCTGGAAATTACACAGCTGTGCAGGTGGCGGGGGTGGCATGAGGA
GGAGTGGATGGCCCACATTCTCGAAGACCTTGGGGAAAACTGGATTAAAA
TGATTTGCCTTATTCTGGTTCTGTAAGATACACATCAGAATGAAACCACC
CCCAGTGTACCTCTGAATTGCTTTTCTATTCTTTTCCCTTAGGGATTTGA
GGGCTTCACTTAGATTTCTCTTCATCTAAACTGTGATGCCCTACATTGAT
CTGATTTACCTAAAATGTCTTTCCTCTCCTTTCAGCTCTGTCCGATCTGG
AGCTCGTGGCCCAATCAATTATCTTTATTTTTGCTGGCTATGAAACCACG
AGCAGTGTTCTCTCCTTCATTATGTATGAACTGGCCACTCACCCTGATGT
CCAGCAGAAACTGCAGGAGGAAATTGATGCAGTTTTACCCAATAAGGTGA
GTGGATG(A/G)TACATGGAGAAGGAGGGAGGAGGTGAAACCTTAGCAAA
AATGCCTCCTCACCACTTCCCAGGARAATTTTTATAAAAAGCATAATCAC
TGATTCTTTCACTGACTCTATGTAGGAAGGCTCTGAAAAGAAAAAGAAAG
AAACATAGCAAATGGTTGCTACTGGCAGAAGCGTAAGATCTTTGTAAAAC
GTGCTGGCTCTGGTTCATCTGCTTTCTATTACTACAATAATGCTAAGTAA
AAAACCTCCAAAAACCTCAGTGGCATCTAACAATAAGCATTTGTTGCTCA
CACTCATTTCAATTGGTTTTGGTTGTGAATTACATGTTTGCAGCAGGCAC
CATAGTGGTGTGTGATGTCCCCTTAGCTGTATCCACATATGGACACAGGA
ATTGGCTCTTTTTATCTCTTTTTATTTTCTTGGTTACAGACATGTGACTT
TTTTTTTTGAAAGGTAACAATCACTTTCTCATATGTTATTTGATGCTAGT
GGTCATAGCCTATAGTCACATTTGTTTCAATGAGAAAGAAAAACCAGTAC
ACGGTTATGCTAAGGATTTCAGTCCCTGGGGTGAGAGCCGTCTCGAATGT
CTCCCCACTTCATAACTCCTCCACACATCATAGTTGGATAGTGAGCTCTG
CTGATATTGGCAGGACTTGCTCTGGTCTGGCTGTAGTCTGACGGAGCCTG
GCCCTGGGTGTGCTGTGCAGGCTGACTCAGCTCTCCCCACACCTATCTCA
TGTTCCAGTCAGGCAGTAACTGGTGAAGAAGCCAAGCTAGGAACCAGGAT
ATCTGGCTCCTGAGCTAAAGTCTTAAAACACTATCATATTGCCTTCCAAA
TATAACACCAAATACTAGGTGCATATCACCCTCACTGTTTTCAGACCTCT
GCCAAAATTGGGATTCTTTGTGGTATGAAGAGACACGGCTTTGGGGCTGG
CCCGGCTGTGACAGTGAGGTGAACACAAAGGGATGTTCTTCAGAGATTAC
AGTCCAGCCCTGAAGCAACAACTA
178 CYP3A7RS2740562

(SEQ ID NO:85)

ATTTTGGTTCTCACAATGTTCACACTCGCTATTGTAAAACAACTTTTTGA
GATTTGTCCTACCAAGGATCCCATATGTCACCAGGTGTTGAGGTCTTCGG
TCTAAACTAGGCTGAGACCATTGTGGTTACCACTTTTCTCCAAGCTTTGG
TGGCCCAGGGACTCCCAGCATCACGTTCTGTCCAGTGTCTGCCTATTCCC
(T/C)TCTTCTTTTTTTCTTCCCTTGATGCCCTTTTATCACATGCATTGT
CTCAGACCCTTCGAATATGTGCTCATAAATGCATGGCATCATCTCCTTCC
CACATCGATTCACTTTCAATTAAAAGCCAAAACTCTTTCATTTCAACTTT
GGATTTAACATGCTTTTGAAAGAAGGGTTGAGAAATATAGAGAAACAGAT
TGGGA
263 CYP3A7RS2687077

(SEQ ID NO:86

AAATCAATGGGATGTTTATTCCCAAAGGGGTGGTGGTGATGATTCCAAGC
TATGTTCTTCATCATGACCCAAAGTACTGGAGAGAGCCTGAGAAGTTCCT
CCCTGAAAGGTAGGAGGCCCCTGGGAAGGGAGCCCTCCCTGAACCAGCCT
GGTTCAAGCATATTCTGCCTCTCTACAGGACAGTCTGGGCTTGTACAATC
(G/A)TTTGCTTGTCTTTTTATGTTTAAAAGGTTTTTTCAAATCATGAAA
TTGATCATTGTCACACTTTACAAACCACAGACTAGATAAAAGAAAACTAT
AGCCAGTCACAGTCCCAGCAACTTAAGATGAAGGTCCTCAATTATGTCCT
TATGGGTCATAAGTGTCCAAAATGTAAGGACTCTTTTAAAAACACATGAT
CACAA
96 CYP3A4_RS2257401

(SEQ ID NO:87)

TCAGGGAGGGCTCCCTTCCCAGGGGCCTCCTACCTTTCAGGGAGGAACTT
CTCAGGCTCT(G/C)TCCAGTACTTTGGGTCATGATGAAGAACATAGCTT
GGAATCATCACCACCACCCCTTTGG
229 CYP3A7RS2687075

(SEQ ID NO:88)

TGCTGTCTCCAACATTCACCAGCGGAAAACTCAAGGAGGTATGAAAATAA
CTTGGGTTTTAATTAGAAACTTAAAGAATGAATCAGGTGGGGACAGGTAG
AAAGTAAGATCAGAGTTCCTTTCCGAGGAGTAGTCTGCTGAATTTGAGCT
TCCTAAAAATAGTCTTTTTATGTACAGAAAACACATCATAAAATTCATTA
(T/C)ACAATGTCACTTATTGTTCCATGCCAGGCAAAGTCATGTCCTTCT
GGGACTTATGTCTGCACATTTAACTATGGGTGGTGTTGTGTTTTGTGCTT
AGATGGTCCCTATCATTGCCCAGTATGGAGATGTGTTGGTGAGAAATCTG
AGGCGGGAAGCAGAGACAGGCAAGCCTGTCACCTTGAAACAGTAAGTAGG
AGCAC
233 CYP3A7RS2687076

(SEQ ID NO:89)

TTTTATGTATATAACAAAAGCCCTGCTTTTGTGNNNNNNNNNNNNNNNNN
NNNNNNNNNNNNNNNNNNNNNNNNNNNNNATACAAATCCTGCTTTGTAAC
TGTTTTTGTTTGTATATATAACAAAAAGAGTTATGAACCAGAAGTTTGGC
CAATAATCCTTGTCGCACAGAGAATTTGCTTTTTCTATCTGTTTTCACTT
(C/T)CTTGGTTACAGACGTGTAACCTCTTTTTTGAATGGTGACAATCAC
TTTGTCATATTTTATTTGATGCTAGTGGTCATAGCCTATTAGTCATGTTT
GCTTCCATGAGAAAGAAAAACCACTACATGGTTATGCTAAGGATTTCAGT
CATTGGGGTTAGAGCCTTCCCGAATGTCTCCTGCTTTCATAACTCCTCCA
CACAT
364 CYP3A7RS2687143

(SEQ ID NO:90)

AACAGGAAGGTTTTTTTTTAAGTTTCAGTGCAAACTCGGAGCAAGTGTCA
TAATTTTCTGTCTCCGATGTGTAGAGGTGACATTTTCTCAGAACTTTCAT
GTTAAGCTGGAAAACTGGAAAGCGAGTCCACTTTGTCATTCTGTCACTCA
CTCATTTTCTCACTCAACAAACATGCCTCACACTTATCTAAATCTGCTAG
(G/A)CTAAAAGAGGTCCCTGGTGTCTGTAACTTTCTAATTCTGCTAGAA
TTCTAGAGTGAGCTCATGAAATAAATGAAAAGGATGAAGAACAAAGAGAA
AAAAGACTGCACGTTCCCTTCTGGCGCTCACTCACATTCCCTCAGCCTCA
GTTTCTCCACATGCCCCTAGAGGTGATCATTCAAGGATTTATGAGATTTT
AGAGA
381 CYP2C9RS2253635

(SEQ ID NO:32)

CTCACAGCAGTACTCCTAAAATTAGATGGCTATAGGATTATACAAATCAC
CTTAATTCTTAACCCCCTAACAGAGTTTATAGCTTATTCCTCCTTCTTAC
TATCCTTTTGAGAAATAATCATAACAAGCTCCTTTTGTCTGTGCCAAACT
GATCTAAAATCACTCATTACTTTTCCATAAGCCACATGGCACTTGTTATC
(A/G)TAGCTTTTCTCATTCAAACCCCCTGAAGTTTTACCAGTGCAACTT
CCCTAATAACTGCCCACAGACTAACCTCATCCCTATTATTCTGCCTTGCA
AACTCAAACTACAAACGAGTTCACAGCCAAACCATACTACTAACACAAGG
CCTTCAAACACTTCTCTCCCTGATAGCCATGTGATGACTCCTAGCTAGTC
TTGCC

These results demonstrate that an inference can be made as to whether a subject is likely to have muscle adverse effects due to treatment with a statin by examining the SNP at positions as indicated in SEQ ID NOS:1-90, above.

### EXAMPLE 2

### Polymorphisms Associated with Response To Treatment with an Ace Inhibitor

This example demonstrates that the examination of the genotype of an individual allows an inference to be drawn as to whether treatment of the patient with an angiotensin converting enzyme (ACE) inhibitor is likely to result in an adverse effect to the subject.

ACE inhibitors are used to reduce the risk of cardiovascular disease, but their use is associated with a dry, persistent cough in a significant number of patients. The cough is very uncomfortable for patients and often is a primary cause of patient non-compliance. Patients experiencing such side-effects are routinely discontinued on ACE inhibitor therapy in favor of Angiotensin receptor blockers, or switched to another ACE inhibitor.

Patients with primary airway disease such as asthma and COPD are at an increased risk of developing cough or bronchoconstriction as a result of ACE-inhibitor therapy (Packard, 2002), and the incidence of the side effect appears to be a function of the genetic constitution of the individual. The metabolism or action of ACE inhibitors have been postulated to cause the generation of nitric oxide (NO) in the bronchial cells, which is a proinflammatory substance on bronchial epithelial cells. Using a randomized, double-blind, placebo-controlled trial, Lee et al. (Hypertension. 38:166-70. (2001)) tested the hypothesis that supplementing iron, an inhibitor of NO synthase, may reduce the cough associated with ACEI use. In this study, iron was used as a supplement in an attempt to ameliorate the cough reaction, which was hypothesized to be a biochemical means by which to counter NO increases. A significant reduction in cough scores was obtained with iron supplementation (P<0.01), but not with placebo. Although iron supplementation can alleviate the adverse effect due to ACE inhibitor treatment, it would be preferable not to have to treat patients with a drug that requires another medicine to counteract potential adverse side effects of the first drug. Instead, it would be preferable to identify those patients at risk for the side effect so that an alternative treatment can be prescribed.

Mukae et al. (J. Hum. Hypertens. 16:857-63 (2002)) identified polymorphisms in the bradykinin B2 receptor gene (−58T/C, exon 1, I/D) that were associated with the cough side effect. Aside from this result, however, little is known about the genetic basis for this adverse effect. Since many ACE inhibitors are metabolized by gene products of the cytochrome P450 system, and possibly other gene products, a candidate gene screen and a whole genome screen were performed to identify markers associated with the cough reaction to two ACE inhibitors; Enalapril (Vasotec®) and Lisinopril (Zestril®).

For the Enalapril and Lisinopril studies described below, a set of 205 xenobiotic metabolism SNPs, including SNPs in the AHR, CYP1A2, CYP2B6, CYP2C8, CYP2C9, CYP2D6, CYP3A4, CYP3A5, GSTM1, GSTM3, HMGCR, MVK, PON1, PON3, CYP1A1, CYP2C19, CYP3A4, CYP3A7, CYP2E1, CYP1B1, CYP2A6, CYP4B1, and GSTP1, was screened.

### Enalapril Study

For Enalapril, CYP2E1, CYP4B1 and CYP2C8 showed multiple hits (see Table 7) The CYP4B1 markers are in linkage disequilibrium (LD) with one another, as are the CYP2E1 and CYP2C8 markers, but the LD is not complete for any of these three genes. The clustering of associated markers to specific gene regions indicates that the associations are not spurious SNP associations, but are associations between a gene region and trait value (adverse response).

TABLE 7
ENALAPRIL PHARMACOS

| Delta | Marker | |
|---|---|---|
| 0.168981 | 198 | CYP2E1RS2070675_TC |
| 0.151416 | 143 | CYP4B1RS2405335_TC |
| 0.141745 | 74 | CYP2E1RS915907_CA |
| 0.135854 | 127 | CYP4B1RS1890251_TC |
| 0.129855 | 362 | CYP2C8_2071426_GA |
| 0.129167 | 132 | CYP4B1RS1890250_TC |
| 0.127594 | 343 | CYP4B1RS751027_GA |
| 0.12605 | 194 | CYP4B1RS681840_TC |
| 0.123532 | 2760 | CYP2C8_2185571_TC |
| 0.119907 | 50 | CYP4B1RS837395_TA |
| 0.118112 | 391 | CYP2C8_RS1934952_GA |
| 0.114815 | 2655 | PON1E6-5_157_869819_TC |
| 0.104562 | 2642 | 2C9E2E3_394_869797_TC |
| 0.098349 | 97 | CYP4B1RS2297812_GC |
| 0.094729 | 207 | CYP2A6RS1809811_TC |
| 0.093649 | 265 | CYP2C8E8_92_GA |
| | 138 | CYP2C8E2E3_248_TC |
| | 176 | CYP4B1RS1572603_TC |
| | 171 | CYP4B1RS632645_TC |

Ranking of the markers shown above based on p-values revealed that most have significant p-values (p<0.05; using the chi-square test or the exact test—see Table 8). As indicated in Table 8, alleles for each of these markers are in Hardy-Weinberg Equilibrium.

TABLE 8
ENALPVALUE

| Marker # | P-ChiSq | ChiSq | P-Exact | HWE:ChiSq | HWE:P-Value |
|---|---|---|---|---|---|
| 198 | 0.0018 | 9.7852 | 0.0018 | 1.1548 | 0.2825 |
| 74 | 0.0063 | 7.4768 | 0.0029 | 0.7565 | 0.3844 |
| 132 | 0.0112 | 6.434 | 0.017 | 1.6281 | 0.202 |
| 343 | 0.013 | 6.1727 | 0.0166 | 1.5588 | 0.2118 |
| 127 | 0.014 | 6.0351 | 0.0252 | 0.4433 | 0.5055 |
| 143 | 0.0142 | 6.0152 | 0.02 | 1.0076 | 0.3155 |
| 50 | 0.0205 | 5.3725 | 0.0354 | 1.1791 | 0.2775 |
| 207 | 0.023 | 5.1693 | 0.0328 | 0.7001 | 0.4027 |
| 194 | 0.0256 | 4.9842 | 0.0435 | 0.5302 | 0.4665 |
| 265 | 0.0264 | 4.9286 | 0.0192 | 0.454 | 0.5004 |
| 138 | 0.0494 | 3.8608 | 0.0323 | 0.3863 | 0.5343 |
| 362 | 0.0617 | 3.4921 | 0.0636 | 3.3024 | 0.0692 |
| 2760 | 0.0721 | 3.2356 | 0.084 | 3.465 | 0.0627 |
| 2655 | 0.0781 | 3.1033 | 0.0945 | 1.7688 | 0.1835 |
| 176 | 0.0892 | 2.8896 | 0.0526 | 1.0068 | 0.3157 |
| 391 | 0.0974 | 2.7483 | 0.1059 | 1.5098 | 0.2192 |
| 171 | 0.0987 | 2.726 | 0.1283 | 0.418 | 0.5179 |

Markers 138, 176 and 171 did not have delta values above the cut-off value, but had p-values near 0.05 and, therefore, are included in the list of sequences useful for inferring an adverse effect due to Enalapril. The SNPs from this screen were used to construct a classification system, whereby patients were given a non-responder score (−1) if they possessed a genotype for a given SNP associated with non-response, and a responder score (+1) if they possessed a genotype for a given SNP associated with response (and no score if they possessed neither). As shown in Table 10, adverse responders (case) were classified with 98% accuracy, and 93% of the time an individual was predicted to be a responder, the individual was, in fact, a responder.

TABLE 10
Classification results Enalapril -
candidate gene segment of the study only.

| | INTO | INTO | |
|---|---|---|---|
| | CASE | CONTROL | |
| CASE | 41 | 1 | 0.97619 |
| CONTROL | 13 | 14 | 0.518519 |
| | 0.759259 | 0.933333 | 0.797101 |

Regions of the genes, including the SNP positions, for markers as set forth in Tables 8 and 9 are set forth below (indicated by “marker name”—see Table 8). The SNP position, and polymorphism, is indicated by brackets [ ].

CYP4B1RS2405335

(SEQ ID NO:91)

CTTGGGCAGGGATTCTTACTGGGTGAAGGTGCTGGGCAGGCACCCTGTGT
GCTGGGAATCCGAGACCGACATGACCTGCCTTGAGGGAACTATGACATGC
TGCCGAAAAGGAAACCCCTTCGTCTTTCTCCTCCCTCACCTTTGTTTTCG
AAACTCTTATCCCCATTTTGTGAGCAGACTGATTTTCATCTGAAGCAGCC
CCTGGGGCACTGTAGCTGCAGCACACTGGTCAAGGGTAGCTTGTTTCTTG
GACACTGGCCCCACTGAGCTTCGGGTCAACTGGCTGATGGCTTGAGCAGG
CTTGCTAACAAGGGCTGCAGAAAGAGACTAAGCCAGGATCCCTGGTCTCC
CTTAACTCA[C/T]GCTGGACTGTTCCCTTTGGTCTTTGTACCGCACGTT
TCCACCTTTAAAGAGAGGTCAGGGTTCCACTAGGCAATGTTGAAATCCTT
TTCCGATCTGATTCTTGGAGCAAAGCTTACTTTAGGCTCTTGAGAAGCAG
AGAGGAAAGTGGTGACGTATGCTTGCTCTTCCCAATCCCTCCAGTGGTTT
ATTTCTGGTGAGGTCAACAAGGAGTGCCAATGTGAGGGTGGGAGGTGGGG
CTGGGGAGCTGGGATCAGTGAATTATTTTCATACCCTGCTCTCTTCCTGT
TTCCTTCCTTCTGGAGAAAAAACAAGCCGTCCCAGAACTTCCAGTCCTGT
AAGTGTCCCAGCTCAGGCATTCTGAGGGCGAGTCTGAGGCATGCAGTTGT
GTAATCCAGAGGAACTGTCCAGGCAGTCACGTGTACCCCACAGGGAGGAT
GCCTCAGCCCTCTTGAGGGGCTGCAGCTGCAATAGTGGGACCAGGTGCTG
GGAGCTGTCCTC
CYP2E1RS2070675

(SEQ ID NO:92)

GCAGACTTTGAGAATTGCATTTTGTCTGAGGAGAAGCCCTCAGCCTTCCT
TGTGGGCATG[C/T]ACTCCCCAACTGTGCGCAGCTGCAGGACTTCCAGG
CCTCCCCAGCTTCATCCACCTGCAG
CYP2E1RS915907

(SEQ ID NO:93)

CGTGCAGGACTTCCAGGCCTCCCCAGCTTCATCCACCTGCAGGTGCTCAG
GATCCTGATCCCCTGCCCCCTTCCCACCTTGGTGAAACTTCTTGTATCCT
TGTCTTGTCCTTTCCTATGGCTTGTGGCTCAAGAACAAATGTGGAGCCCA
CACTGATTTCCCAGGACTGTCTGAGCATCTTCTCCACCAGTTTGGCCCCT
CGTGGCAGCAGA[A/C]ACTAGCCCTGTAGCAGGAGGGGTTAGCAGGAGC
CGTTTAGCTCCTGCCTGAGCTATGACCAAGGTCAGGGGGATCTCACCTCT
CCCAGGATGGCCCTCATGCTGTGGAGGGAGACAGAGCCCTGGCCTGCCCT
CAGCAGATTTCTGGGAGCCTCAGTTTCCCTGGCTGTGAGTGGAGATGACT
CTGTCTGTCACAGCTCCAAGTCACAGTTCCACTGGGAGAGCCTCTTGGAC
ACTGTCTCCTGTGTCCCTGTGGAGCTGGGAGGTGGCTGGTTCTGTGCTGA
AAGGAGACAAGCAGCCCCTTCTCTCCGGTCTGTCTCCGGTATCACAGGAA
AAGCACAGTGCAGAGCGCTTGTACACAATGGACGGTATCACCGT
CYP4B1RS1890251

(SEQ ID NO:94)

TCCACCCACAGGCAAGGGGAAACATTCTTGTATGCCTGCCTCCCTCCTGG
CTGGTATCCCTGGAAGTGCTCCTTTCCCTGGTGTCTGTCTTATTTTTCCA
TCTGCCCTCCTGGGTTTTCCTTGCCAACTTCCTGGTCACCTGGCCATTCC
CCTTCTCTCCATCCCTACCAGGTCCAGAAAGCCCAATTGGATGGGTCAAG
GAAAGGCTTAAGTCACAAAAAAGCCNNNNNNNNNNNNNNNNNNNNNNNNN
NNNNNNNNNNNNNNNNNNNNNNNNNTCCAAGCCCCACTTCCCTCCTTATC
AGCTATGCTTGTGCACCTCTGCAGCAACCCCACAAGGGACAGTAGACCCT
GGTCTCATGCTGACCACATTCACAACCTGTGCCCTGATCGAGGTCGCAGC
CAGCTTGCCACTGCCTCCCTGTGCCCAGCTTTCTCCTAGCTCTTTAGATT
TATTTAAGAAATAAAGAAAACTAAGATAAATAAAAAACATGTGCATGAGC
CCAAGCGATGGCCAAACTTTTAAATAAAG[C/T]TAGAGAACTAGAAGCA
CCTGCCAGGCACCTGCCTTGATTACCCCGGGAGCCTGGTAAGAAGGAGGA
CAGCTCCCAGCACCTGGTCCCACTATTGC
CYP2C8_2071426

(SEQ ID NO:95)

TTTGTCTTTCTAAGTCTGTCATTGTCAGAAATAGCGGAGTGAGTTGATGC
ATTTTGTGAATACAGAAACATTGGGGTCATTGTATTATATAATCATTTAA
TACAGTGGCAAAAGTTTAAAGTGCTGTTTCTCCTCTTTGTTTCACAGTGT
TTTGCTATGATTTTTGACTGAAGGTGAAGGGAAGTGTGTGTGATTAGAAA
TTTCATCCA[A/G]TAAGTTCTCTACTATAGTAGTCATGTGTTTTATTCA
GAATGGTCATGAAAATTGAACTTCTCTGAAGATTCATTTGATGGCTGATG
TGAAATAAATATCTGTGGGTTCAGGGCAAACATAAGTGCATGAAAGAAAG
AAGTAATCAGTCAGGGCCCAATAGGTAGTTAACAGAATTCTTTTGGATTC
TGAAGAAAGCCACTGTCTGTGGCCAAGGTTGCTGGAGAATGGAAGAAATT
GTTCTTCCAGGAGATGCTGAATGTCCTGATTCTAACTTTGTGGTGCTTCA
TCGTTCCATATTGGTAATAC
CYP4B1RS1890250

(SEQ ID NO:96)

TCCACCCACAGGCAAGGGGAAACATTCTTGTATGCCTGCCTCCCTCCTGG
CTGGTATCCCTGGAAGTGCTCCTTTCCCTGGTGTCTGTCTTATTTTTCCA
TCTGCCCTCCTGGGTTTTCCTTGCCAACTTCCTGGTCACCTGGCCATTCC
CCTTCTCTCCATCCCTACCAGGTCCAGAAAGCCCAATTGGATGGGTCAAG
GAAAGGCTTAAGTCACAAAAAAGCCNNNNNNNNNNNNNNNNNNNNNNNNN
NNNNNNNNNNNNNNNNNNNNNNNNNTCCAAGCCCCACTTCCCTCCTTATC
AGCTATGCTTGTGCACCTCTGCAGCAACCCCACAAGGGACAGTAGACCCT
GGTCTCATGCTGACCACATTCACAACCTGTGCCCTGATCGAGGTCGCAGC
CAGCTTGCCACTGCCTCCCTGTGCCCAGCTTTCTCCTAGCTCTTTAGATT
TATTTAAGAAATAAAGAAAACTAAGATAAATAAAAAACA[C/T]GTGCAT
GAGCCCAAGCGATGGCCAAACTTTTAAATAAAGCTAGAGAACTAGAAGCA
CCTGCCAGGCACCTGCCTTGATTACCCCGGGAGCCTGGTAAGAAGGAGGA
CAGCTCCCAGCACCTGGTCCCACTATTGC
CYP4B1RS751027

(SEQ ID NO:97)

GTTTATATCAATATGAAGAGGGATGCCCTGTGGAGGCAGCCCCTCCTCCC
TGCAGGCCCACCAAGTGATTTTTACATTGAAATCAGCAAACCAGAGCAAA
AAGAACCAGATGTAGCAGGTCATGGGAGGAGACTCTGCCACGGAATTCTC
CAAATCCAGTACTCGAGGATCCCATACCCAGACACTGACAGGTGCTGCCC
[G/A]CCACTGAGCCTCCTCTTTCTGGGTCTCAGATGCCCACATTTTAAA
ATTGAGACATAGAAATTCATTCCTCCTGTTTACAATCCAGTCTTATGGCT
CTCCTTTGATGACTTTCGCTAGATTCTGCTCTAGTCCAGCTGTGTTGATG
CCTCCAATTAACAGAGCTCCAAGGGTAATCCTTGTTTCCTTTTCCTGCTA
CATGG
CYP4B1RS681840

(SEQ ID NO:98)

GCCCCAGGGTGTTTTGTAGCAAGAATCCCTCTATGATGATTTAACACCTG
ACGTTTATCTGCCCAGTAGTTTGCAAAATACCTATACTTTATCTCTTTCT
ATCCCCTTCTNNNNNNNNNNNNNNNNNNNNNNNNNNNNNNNNNNNNNNNN
NNNNNNNNNNNNNNNNNNNNNNNNAAAGTGGCTTAGCTAACTGGGCACAG
[C/T]GAAGGCCTGTAATCAATGTTTACTGAATGAAAATGAGCCACAGCC
CTGTCTTCACTACACAGCACAAGGTCAAGGGCACTCTCACAGCAAAGGGC
ATGGCTGGGAAAGAGGCCTCTGCAGTCTCAGAGTATCTCATTTCCTTGAT
CTGGGAGCTGGCATTCT
CYP2C8_2185571

(SEQ ID NO:99)

TTAGGACCTATAGATGTGAATTTGGGCACCCTTTCCATTTATGTTCACTA
GTCACTTGGTTACAGCAATTTCATTTTAGAGATTCCTTCACCGAATAAAA
GCCTTTTTAATAATTTCTTTACCTGTAAAATTACAGATTTGGCCTAACTT
ATGGTCAAATTTAAAACTTTATTAAAACATACTTATCTTGATGACTATAC
TGAATAATATCCCAGAATCCACAAGAGTAGTTTTGCCCAAATGAATTAAA
TTTGGAAGATGATTGGAAATGATAAACATTATTTCCAAAGGAAGGAAATT
CATTTTTTACAAATTGAAAAAATAAATAAAAAGATTCCATTTATCAATTT
ACTTTTTGGTTAGCTTTACTGGGATTTAGATTTGAAATAAAAAGTTAGCA
TTTTACTTATTTAGATAAACAAATCTGTTATACTTAATGCTCATTTATAT
TAGTTTTTCTTCTCATATCATATGGATGTACTTGCTTTTCACATTTGTTC
CCTAAAACACATGAATTCACCCACAATCTCCCTAACTTTATTCCCATTGG
TTCAGCACATTATACTTTCAGCTATATGACATTTCTAGGTCCCCAACTTT
TCTCTTCCTATCTCTGTGTTTTATTGTCCAGAGGCTTTGCAAACAGACAT
GATTCTGCTCTAAAAACTTGAATTATTAGGATTTCAAAAAAGTCTTCTTT
TTCCTTCCAAAATAAAAGACACTTGG[A/G]GTTAAAAGAGTTTAAATAA
AGGCAGTGGATGTGAATAACCACATGCGAAATGACATTGTTTAGCTAGCC
TGTAATGCTCAACTCATATTTAAGGTAAAAGTAATGTGTTTATTTCATCC
ATGCTGATTTTTTTTGGACACATGGGGAATTTGTAAGATATTGTTTAAAA
TTTCTAAATTTCCTTTATGTCTTAACAAATGCAAATC
CYP4B1RS837395

(SEQ ID NO:100)

CCCACCCAAAAACTAGCCAGGAATATGGAATGTCGTTTTCTCCATGGTTT
ATATCAATATGAAGAGGGATGCCCTGTGGAGGCAGCCCCTCCTCCCTGCA
GGCCCACCAAGTGATTTTTACATTGAAATCAGCAAACCAGAGCAAAAAGA
ACCAGATGTAGCAGGTCATGGGAGGAGACTCTGCCACGGAATTCTCCAAA
[T/A]CCAGTACTCGAGGATCCCATACCCAGACACTGACAGGTGCTGCCC
GCCACTGAGCCTCCTCTTCCTGGGTCTCAGATGCCCACATTTTAAAATTG
AGACATAGAAATTCATTCCTCCTGTTTACAATCCAGTCTTATGGCTCTCC
TTTGATGACTTTCGCTAGATTCTGCTCTAGTCCAGCTGTGTTGATGCCTC
CAATT
CYP2C8_RS1934952

(SEQ ID NO:101)

CTTGCTTTTCACACCTGTGTTTTGTGTGCTAAGTCTCTGGCCATGGAATC
TCCTTCCTTTATTCTTTGATCTTTTATGTCTGCTTTATATTTGGCACTGT
AGATACCAACATGACAAGGACAGAGACCTTCCTTCAAGAATTCACATTTT
ACCACAATAGATAAATAAATACAGAATTACGATTCCTGGTAATCTGTGCT
TTAATGAAAGCATGAGTATGCTGTTGAGGAAAACCAGAGAGCATAGGATT
TGATTACTTGAGGGGATTGTAAAGAGTGTGAAGATGAAATGAAATTGAAC
CTATGTCCTGAATGTAGCCTAATAAAGATGTATCTTAGCCAAAATGAACA
ACAGGATATGAAACTTGAAACTTCATGGTGTGTTCAAGATTGATGAAGAG
AGTGTATGACCAGAGCTGA[A/G]AACAGATTATGTGAAAAGTGGAAGGA
AAAGTCATGGAATATCAGGCTTGGAACAGGAAACAAGAATGTGCATGTAT
TTCCATAATGACAGATGGAAACTCAAAATGGCAAAAGAGCTGTTTTAATG
TGGGAATAAATAA
PON1E6-5_157_869819

(SEQ ID NO:102)

AGAATATATTCCAAGATATCTCCTGAGAATCTGAGTAAATCCACTACATT
TCAGAGAGTTCACATACTTGCCATCGGGTGAAATGTTGATTCCATTAGCA
AAATCAAATCCTTCTGCCACCACTCGAACTTCACTTGGACTATAGTAGAC
AACATACGACCACGCTAAACCCAAATACATCTCCCAGGAT[C/T]GTAAG
TAGGGGTCAAGAAAATAGTGATCATTTGTGCCATAAAAGTGCTCAGGTCC
CACAGCAACAATATCATTCAAACTGCAATTAAAACATACACACATAATAT
ATAAGGTGAAGGTATTGAACATTACAGGATTATTAACTGGCATTCCTCAC
TGTC
2C9E2E3_394_869797

(SEQ ID NO:103)

TGATTGATCTTGGAGAGGAGTTTTCTGGAAGAGGCATTTTCCCACTGGCT
GAAAGAGCTAACAGAGGATTTGGTAGGTGTGCAWGTGCCTGTTTCAGCAT
CTGTCTTGGGGATGGGGAGGATGGAAAACAGAGACTTACAGAGC[C/T]C
CTCGGGCAGAGCTTGGCCCATCCACATGGCTGCCCAGTGTCAGCTTCCTC
TTTCTTGCCTGGGATCTCCCTCCTAGTTTCGTTTCTCWTCCTGTTAGGAA
TTGTTTTCAGCAATGGAAAGAAATGGAAGGAGATCCGGCGTTTCTCCCTC
ATGACGCTG
CYP4B1RS2297812

(SEQ ID NO:104)

ACGATACGGCAGGGGTGGAATTGAGGCCATTTCTCTATTGCACAATGAGC
AAATATTACCTATCTTTATAATTAAAGAGCTTTAATGTGGAGAAGAAAGC
ACTTTCTGGCTGCAGTGCAGTAGAGAGTTGGTGGCAGGTGCTAAATGGAA
GCAGGTTGGCTACTTTGGGGATGTTGAGCTAACCCCGGTGTGAGAAGAGG
GCGGCTTGGAATTAGAGCAGTGGTTGTGGGGCTGGAGAGGGGACTAAGCC
CAGGGATGTTTAGGAGAAGAGGGTGCAGGAGAGGTAAGGAAGCAACCAGG
GCCTGCCTGGGCAGCCTTCTCCCCTGCCCTCCCAGGGACTTGGGGCCTAG
CTGGTGACAATGTGTTCCTGAGTGACCTTGGCCTTCTGTCATCATTTCAG
ATCCAGGAGACGGGGAGCCTGGACAAAGTGGTGTCCTGGGCCCACCAGTT
CCCGTATGCCCACCCACTCTGGTTCGGACAGTTCATTGGCTTCCTGAACA
TCTATGAGCCTGACTATGCCAAAGCTGTGTACAGCCGTGGGGGTGAGGAG
AGAGGATGGGGATCTCAGGAGAGGGTGGGGCTTCCTGAGAACAAAGGGCT
CAGGGCATGATATGGGGAGGAAGCCTGGGCCTGTGTACTAAGTCTGCG
[G/C]AGCTGAGGTTCCCACCCTACTCATAAATGAGCCTCCTCTAGGAAC
CCCGGGTCCCTGCTTGACCTGATTGTCTCCTCCTGCAGACCCTAAGGCCC
CTGATGTGTATGACTTCTTCCTCCAGTGGATTGGTGAGTGAGCACCTGCC
TTCCCTGCCCTGCCAACCTCAGACCCGTGGTGCTGGGTGACTAGGATCCT
GGCCTGTCCCACTCAATTGGTTATCCCAGATGGCCTAGTTCTCGGGTGCC
CATCCTAAGCTCAGCTGCTCAGTGGAGAGACAAGGGAGGAGCAGGAGAGC
CCCCAGCTGTGGGGCCAAGGCATCTTCTCTGGCAGGGCCTGATTCTCTAG
ACAGGGCAAAGGCTTTGGAGAATGTGTGAGTGCGAAGAACAGTTTCCTGA
AGGAGGTGGCACTAGAGTTGATCCAAAAGGAGATTTAAATAGGTAGTGCT
GGCCAGGGCAAGGGCCTAAGGAAATGGTGTGGTGGTAGGACCACGGCTGG
TCACCAGAGGCTGTGAGGCTGTCGGGGTGGCTACAAGGCAGAGCCC
CYP2A6RS1809811

(SEQ ID NO:105)

TGTTCCCCAACCCTTTCCAATCACACACCTTACAGAGGTGCTCCCAGTGC
ATCCCACTTGGATAGGTAAACACCTCAACAGGTATCCCCTCCACTTCAAC
ATCTTCACCAGCCCCACTTTAATACCTGAGCACCTGAACAAAAGCCCCCA
ATCCAGACCCAGTAAGTATCTGGACAGCTGTCTCCAACCAAGCCTACTTG
AATGCCTAAATACCTAGACAGGTGACACTCACCTCATACCAGCCCCACCT
GAAGAGCTAAACACCTGGACAGGTGTCTTCCAACTCAACTTCACTTGAAT
ATCTGAACACCTAGATGTGTGCTCCAATCCAGCCTCGTTTAAATACCTGA
AACCTGGATATATGTCTCAGTTCTTCTCACCTAAATTACTAGACCGTGGC
CCTGGTACCTAACCTTCCTGAAAACTTAGATATAAGTTCCTATCCGGCCC
CACTGAAATACCTAAACAACGAGACAGATGCCTTTAACTCAGTTCCTTCC
TTGCTATGAAACAAATCCCATTCCCATCAGCTCCTGCCCCGTGACAGCTG
TCCTTCCCTTCCCATCCTCTCTCTGCAACCCCAGCTCTATGAGATGTTCT
CTTCGGTGATGAAACACCTGCCAGGACCACAGCAACAGGCCTTTCAGTTG
CTGCAAGGGCTGGAGGACTTCATAGCCAAGAAGGTGGAGCACAACCAGCG
[C/T]ACGCTGGATCCCAATTCCCCACGGGACTTCATTGACTCCTTTCTC
ATCCGCATGCAGGAGGTACACCCCAGCAGCCACTGCGGGGAGATGCAAAG
CCAGGCAGAGGGAAATCAGTCTGGGAGTGGGGCAGGCAGATGACACAGGC
CCATTCAAATTAACCCTCATCATAATAATCCTCACAATT
CYP2C8E8_92

(SEQ ID NO:106)

TTCTTATAATCAGATTATCTGTTTTGTTACTTCCAGGGCACAACCATAAT
GGCATTACTGACTTCCGTGCTACATGATGACA[A/G]AGAATTTCCTAAT
CCAAATATCTTTGACCCTGGCCACTTTCTAGATAAGAATGGCAACTTTAA
GAAAAGTGACTACTTCATGCCTTTCTCAGCAGGTAATAGAAACTCGTTTC
CATTTGTATTTAAAGGAAAGAGAGAACTTTTTGGAATTAGTTGGAATTTA
CATGGCACCTCCTCTGGGGCTGGTAGAATTGCTA
CYP2C8E2E3_248

(SEQ ID NO:107)

ATCTCCCTCCACCACCTGAGGGCTGACAACCAGGATGCGCAATGAAGACC
TGGCCACCCCTGAAATGTTTCCAAGGACGTCACTAGTGAAGACAGGTAAC
TGTTAAGGTCAATGACGCAGAGTAGAGTCACCCACCCTTGGTTTTTCTCA
ACTCCTCCACAAGGCAGTGAGCTTCCTCTTGAACACGGTCCTCAATGCTC
[C/T]TCTTCCCCATCCCAAAATTCCGCAAGGTTGTGAGGGAGAAACGCC
GGATCTCCTTCCATCTCTTTCCATTGCTGGAAATGATTCCTAATAAAAAA
AGGGGCAGAAACTGGGAGAATTCACAGCCAAGGAAGAAAGTGCTGCAACA
CTCGGCAGCCATGCAGATAGGCTAAGCTCTGCTGAGAAGCTTTTTAGGGC
TCTG
CYP4B1RS1572603

(SEQ ID NO:108)

GAAGGAGGACAGCTCCCAGCACCTGGTCCCACTATTGCAGCTGCAGCCCC
TCAAGAGGGCTGAGGCATCCTCCCTGTGGGGTACACGTGACTGCCTGGAC
AGTTCCTCTGGATTACACAACTGCATGCCTCAGACTCGCCCTCAGAATGC
CTGAGCTGGGACACTTACAGGACTGGAAGTTCTGGGACGGCTTGTTTTTT
CTCCAGAAGGAAGGAACAGGAAAGAGAGCAGGGTATGAAAATAATTCACT
GATCCCAGCTCCCCAGCCCCACCTCCCACCCTCA[C/T]ATTGGCACTCC
TTGTTGACCTCACCAGAAATAAACCACTGGAGGGATTGGGAAGAGCAAGC
ATACGTCACCACTTTCCTCTCTGCTTCTCAAGAGCCTAAAGTAAGCTTTG
CTCCAAGAATCAGATCGGAAAAGGATTTCAACATTGCCTAGTGGAACCCT
GACCTCTCTTTAAAGGTGGAAACGTGCGGTACAAAGACCAAAGGGAACAG
TCCAGCATGAGTTAAGGGAGACC
CYP4B1RS632645

(SEQ ID NO:109)

CTTGCCTATGCTTTCTCTCAAGAAAGCTTCCAGTGTCCCCTTCAATGTGA
ACCCTTAAGAGAGCTGGCATTTATGCTAGGCTCGCAATGCTTTGAGTTCT
TTTTTGTGAGGCACCTTCAGAGACAAGGTTCTAGCCCCAAAAGGGAAATA
CCAGG[T/C]CAGAAAGGGGCCAACTCCACCCCTAAAACAATAGTGCCAT
TTTGACACTTAGGAACATAGAACTTTCAGGGTGATG

### Lisinopril Study

For Lisinopril, CYP2D6, CYP2B6 and PON1 showed had multiple “hits” (see Table 11). Markers within each of these three genes were in LD, but the LD was not complete and the information carried by one was not completely redundant with the information carried by another. The clustering of associated markers to specific gene regions showed that the associations are not spurious SNP associations, but are an association between a gene region and trait value (adverse response).

P-values for some of the markers are shown below in Table 12 (LISINPVALUE), which provides a ranking of the top p-values from all of the 205 xenobiotic metabolism markers screened. The sequences for all of the SNPs in Table 11 are included in the list below.

TABLE 11
LISINOPRIL
LISINOPRIL PHARMACOS

| Delta | Marker | |
|---|---|---|
| 0.19697 | 2620 | 2D6E7_286_554365_CA |
| 0.171875 | 423 | CYP2D6_RS769261_GT |
| 0.171875 | 2699 | CYP2D6_RS2856955_GA |
| 0.166667 | 2700 | CYP2D6_RS2854740_GA |
| 0.151515 | 324 | CYP2B6RS2279343_GA |
| 0.141711 | 188 | CYP2B6RS1042389_TC |
| 0.140625 | 2709 | CYP2D6_RS2743459_GT |
| 0.121212 | 73 | CYP1A2_RS762551_CA |
| 0.107143 | 2686 | PON1E2-5_328_869817_GA |
| 0.101604 | 2618 | PON13UTR_eye_886925_CA |
| 0.100267 | 330 | CYP7PE1_580_GA |
| 0.093137 | 58 | GSTP1E2E4-5_419_CA |
| 0.092979 | 172 | CYP2D6_RS2267446_TC |
| 0.091398 | 205 | CYP2E1RS2480259_TC |
| 0.090909 | 2672 | CYP2D6_RS2004511_GA |

TABLE 12
LISINPVALUE

| Marker # | P-ChiSq | ChiSq | P-Exact | HWE:ChiSq | HWE:P-Value |
|---|---|---|---|---|---|
| 2620 | 0.021 | 5.3279 | 0.0333 | 0.0299 | 0.8627 |
| 324 | 0.0423 | 4.125 | 0.0623 | 0.2398 | 0.6244 |
| 2699 | 0.0437 | 4.0683 | 0.0578 | 1.5219 | 0.2173 |
| 423 | 0.0484 | 3.8964 | 0.0762 | 2.00E−04 | 0.99 |
| 2700 | 0.0539 | 3.7167 | 0.0788 | 0.1104 | 0.7397 |
| 188 | 0.057 | 3.6235 | 0.0755 | 0.4539 | 0.5005 |
| 2618 | 0.0901 | 2.8723 | 0.0752 | 0.645 | 0.4219 |
| 2709 | 0.0989 | 2.7234 | 0.1489 | 0.5088 | 0.4757 |

Based on the SNPs identified from this screen, a classification system was constructed, whereby patients were given a non-responder score (−1) if they possessed a genotype for a given SNP associated with non-response, and a responder score (+1) if they possessed a genotype for a given SNP associated with response (and no score if they possessed neither). As shown in Table 13, responders (control) could be classified with 96% accuracy, and 93% of the time an individual was predicted to be a non-responder (case), the individual was, in fact, a non-responder.

TABLE 13
Classification results Lisinopril -
candidate gene segment of the study only.
haplo counted once method 19 incons

| | into | into | |
|---|---|---|---|
| | case | control | |
| case | 13 | 11 | 0.541667 |
| control | 1 | 23 | 0.958333 |
| | 0.928571 | 0.676471 | 0.75 |

Regions of the genes, including the SNP positions, for markers as set forth in Tables 10 and II are set forth below (indicated by “marker name”—see Table 10). The SNP position, and polymorphism, is indicated by brackets [ ].

2D6E7_286_554365

(SEQ ID NO:110)

AGCCCATCTGGGAAACAGTGCAGGGGCCGAGGGAGGAAGGGTACAGGCGG
GGGCCCATGAACTTTGCTGGGACACCCGGGGCTCCAAGCACAGGCTTGAC
CAGGATCCTGTAAGCCTGACCTCCTCCAACATAGGAGGCAAGAAGGAGTG
TCAGGGCCGGACCCCCTGGGTGCTGACCCATTGTGGGGACGCRTGTCTGT
CCAGGCCGTGTCCAACAGGAGATCGACRACGTGATAGGGCAGGTGYGGYG
ACCAGAGATGGGTGACCWGGCTCRCATGCCCTRCAYCACTGCCGTGATTC
AYGAGGTGCAGCGCTTTGGGGACATCGTCCCCCTGGGTGTGACCCATATG
ACATCCCGTGACATCGAAGTACAGGGCTTCCGCATCCCTAAGGTAGGCCT
GGCRCCCTCCTCACCCCAGCTCAGCACCAGC[A/C]CCTGGTGATAGCCC
CAGCATGGCYACTGCCAGGTGGGCCCASTCTAGGAAMCCTGGCCACCYAG
TCCTCAATGCCACCACACTGACTGTCCCCACTTGGGTGGGGGGTCCAGAG
TATAGGCAGGGCTGGCCTGTCCATCCAGAGCCCCCGTCTAGTGGGGAGAC
AAACCAGGACCTGCCAGAATGTTGGAGGACCCAACGCCTGCAGGGAGAGG
GGGCAGTGTGGGTGCCTCTGAGAGGTGTGACTGCGCCCTGCTGTGGGGTC
GGAGAGGGTACTGTGGAGCTTCTCGGGCGCAGGACTAGTTGACAGAGTCC
AGCTGTGTGCCAGGCAGTGTGTGTCCCCCGTGTGTTTGGTGGCAGGGGTC
CCAGCATCCTAGAGTCCAGTCCCCACTCTCACCCTGCATCTCCTGCCCAG
GGAACGACACTCATCACCAACCTGTCATCGGTGCTGAAGGATGAGGCCGT
CTGGGAGAAGCCCTTCCGCTTCCACCCCGAACACTTCCTGGATGCCCACG
GCNNNNNNNNNNNNNNNNNNNNNNNNNNNNNNNNNNNNNNNNNNGCCTGT
GGGGAGCCCGGCTCCCTGTCCCCTTCCGTGGAGTCTTGCAGGGGTATCAC
CCAGGAGCCAGGCTCACTGACGCCCCTCCCCTCCCCACAGGCCGCCGTGC
ATGCCTCGGGGAGCCCCTGGCCCGCATGGAGCTCTTCCTCTTCTTCACCT
CCCTGCTGCAGCACTTCAGCTTCTCGGTGCCCACTGGACAGCCCCGGCCC
AGCCACCATGGTGTCTTTGCTTTCCTGGTGAGCCCATCCCCCTATGAGCT
TTGTGCTGTGCCCCGCTAGAATGGGGTACCTAGTCCCCAGCCTGCTCCCT
AGCCAGAGGCTCTAATGTACAATAAAGCAATGTGGTAGTTCCAACTCGGG
TCCCCTGCTCACGCCCTCGTTGGGATCATCCTC
CYP2D6_RS769261

(SEQ ID NO:111)

TGACTCTTGCAAGGTCATACCTGGGTGACGCATCCAAACTGAGTTCCTCC
ATCACAGAAGGTGTGACCCCCACCCCCGCCCCACGATCAGGAGGCTGGGT
CTCCTCCTTCCACCTGCTCACTCCTGGTAGCCCCGGGGGTCGTCCAAGGT
TCAAATAGGACTAGGACCTGTAGTCTGGGG[G/T]GATCCTGGCTTGACA
AGAGGCCCTGACCCTCCCTCTGCAGTTGCGGCGCCGCTTCGGGGACGTGT
TCAGCCTGCAGCTGGCCTGGACGCCGGTGGTCGTGCTCAATGGGCTGGCG
GCCGTGCGCGAGGCGCTGGTGACCCACGGCGAGGACACCGCCGACCGCCC
GCCTGTGCCCATCAC
CYP2D6_RS2856955

(SEQ ID NO:112)

CGCGCTATGGGCCCGCGTGGCGCGAGCAGAGGCGCTTCTCCGTGTCCACC
TTGCGCAACTTGGGCCTGGGCAAGAAGTCGCTGGAGCAGTGGGTGACCGA
GGAGGCCGCCTGCCTTTGTGCCGCCTTCGCCGACCAAGCCGGTGGGTGAT
GGGCAGAAGGGCACACAGCGGGAACTGGGAAGGCGGGGGACGGAGAAGGC
[A/G]ACCCCTTACCCGCATCTCCCACCCCCAGGACGCCCCTTTCGCCCC
AACGGTCTCTTGGACAAAGCCGTGAGCAACGTGATCGCCTCCCTCACCTG
CGGGCGCCGCTTCGAGTACGACGACCCTCGCTTCCTCAGGCTGCTGGACC
TAGCTCAGGGAGGGATCGAAGGAGGAGTCGGGCTTCCTGCGCGAGGTGCG
GAGCA
CYP2D6_RS2854740

(SEQ ID NO:113)

TGAGTGGAAAGGTGGTCAGGGTGGGCAGAGACGAGGTGGGGCCAAACCCC
GCCCCAGGCAGGGGAGCAATGTGGGTGAGCAAAGAGTGGGCCCTGTGCCC
AGCTGGACCGGGCTAGGGACTGCGGGAGACCTTGTGGAGCGCCAGGGTTG
GAGTGGGTGGCGGAGGGTGGGGCCAAGGCCTTCATGGCAACGCCCACGTG
[C/T]CCGTCCCGCCCCCAGGGGTGTTCCTGGCGCGCTATGGGCCCGCGT
GGCGCGAGCAGAGGCGCTTCTCCGTGTCCACCTTGCGCAACTTGGGCCTG
GGCAAGAAGTCGCTGGAGCAGTGGGTGACCGAGGAGGCCGCCTGCCTTTG
TGCCGCCTTCGCCGACCAAGCCGGTGGGTGATGGGCAGAAGGGCACACAG
CGGGA
CYP2B6RS2279343

(SEQ ID NO:114)

GTTCAGAGGCAGAGGGGAGTGGGGAAGTGGGGTTCCCATGGAGGGATTGG
GGCCCAGGAGGCGCTCTCTCCCTGTGACCTGCTAGCTCAGCCCTAGGCAA
ACCTCACCACCCCTTCTTTCTTGCAGCTGTTTGAGCTCTTCTCTGGCTTC
TTGAAATACTTTCCTGGGGCACACAGGCAAGTTTACAAAAACCTGCAGGA
AATCAATGCTTACATTGGCCACAGTGTGGAGAAGCACCGTGAAACCCTGG
ACCCCAGCGCCCCCA[A/G]GGACCTCATCGACACCTACCTGCTCCACAT
GGAAAAAGTGGGGTCT
CYP2B6RS1042389

(SEQ ID NO:115)

AGACCACAGTCACACACTGCTGTAGTCTTCCCCAGTCCTCATTCCCAGCT
GCCTC[C/T]TCCTACTGCTTCCGTCTATCAAAAAGCCCCCTTGGCCCAG
GTTCCCTGAGCTGTGGGATTCTGCACTGGTGCTTTGGATTCCCTGATATG
TTCCTTCAAATCTGCTGAGAATTAAATAAACATCTCTAAAGCCTGACCTC
CCCACGTCAAGAGGTGATCTGTGCCATTTTGTGTGTGATTCTTTTATTGT
CGGGTCTCTAGGGATTTTTCTGGAAGGAA
CYP2D6_RS2743459

(SEQ ID NO:116)

CTTCAGTCCCTCCTGAGCTAGGTCCAGCAGCCTGAGGAAGCGAGGGTCGT
CGTACTCGAAGCGGCGCCCGCAGGTGAGGGAGGCGATCACGTTGCTCACG
GCTTTGTCCAAGAGGCCGTTGGGGCGAAAGGGGCGTCCTGGGGGTGGGAG
ATGCGGGTAAGGGGTTGCCTTCTCCGTCCCCCGCCTTCCCAGTTCCCGCT
[G/T]TGTGCCCTTCTGCCCATCACCCACCGGCTTGGTCGGCGAAGGCGG
CACAAAGGCAGGCGGCCTCCTCGGTCACCCACTGCTCCAGCGACTTCTTG
CCCAGGCCCAAGTTGCGCAAGGTGGACACGGAGAAGCGCCTCTGCTCGCG
CCACGCGGGCCCATAGCGCGACAGGATCACCCCTGGGGGCGGGACGGGCA
CGTGG
CYP1A2_RS762551

(SEQ ID NO:117)

TCAAGCACCTGCCTCTACAGGTACCTTTCTTGGGACCAATTTACAATCTC
TGGGATCCCCAACTATAGAACCTGGAAGCTAGTGGGGACAGAAAGACGGG
GAGCCTGGGCTAGGTGTAGGGGTCCTGAGTTCCGGGCTTTGCTACCCAGC
TCTTGACTTCTGTTTCCCGATTTTAAATGAGCAGTTTGGACTAAGCCATT
TTTAAGGAGAGCGATGGGGAGGGCTTCCCCCTTAGCACAAGGGCAGCCCT
GGCCCTGGCTGAAGCCCAACCCCAACCTCCAAGACTGTGAGAGGATGGGG
ACTCATCCCTGGAGGAGGTGCCCCTCCTGGTATTGATAAAGAATGCCCTG
GGGAGGGGGCATCACAGGCTATTTGAACCAGCCCTGGGACCTTGGCCACC
TCAGTGTCACTGGGTAGGGGGAACTCCTGGTCCCTTGGGTATATGGAAGG
TATCAGCAGAAAGCCAGCACTGGCAGGGACTCTTTGGTACAATACCCAGC
ATGCATGCTGTGCCAGGGGCTGACAAGGGTGCTGTCCTTGGCTTCCCCAT
TTTGGAGTGGTCACTTGCCTCTACTCCAGCCCCAGAAGTGGAAACTGAGA
TGATGTGTGGAGGAGAGAGCCAGCGTTCATGTTGGGAATCTTGAGGCTCC
TTTCCAGCTCTCAGATTCTGTGATGCTCAAAGGGTGAGCTCTGTGGGC
[A/C]CAGGACGCATGGTAGATGGAGCTTAGTCTTTCTGGTATCCAGCTG
GGAGCCAAGCACAGAACACGCATCAGTGTTTATCAAATGACTGAGGAAAT
GAATGAATGAATGTCTCCATCTCAACCCTCAGCCTGGTCCCTCCTTTTTT
CCCTGCAGTTGGTACAGATGGCATTGTCCCAGTCTGTTCCCTTCTCGGCC
ACAGA
PON1E2-5_328_869817

(SEQ ID NO:118)

CTTCCTCTCACATACATACCGATTCCTTTAACTAAATTACAGTTAGRAAG
TTCTACGGGTTGTACCTCTCGGAGAGCATTAAGTCGTGTTCTGTGGGGGA
GAAAGAAATAAAACACACACAAAACTATTCAGAAATTATAATAAGTTGCA
AAGGTAGGCAGTCCACAAAAGTTCTCCTTCACT[A/G]TACTTTGCCTGA
GTTTCAACTCCAGAGTTTTTCCTGCAATTTTTCCAGGCAAGGCCAGGGCA
AGACAAGTGGGGAMCTCTGGGTACAATATTTAGTTTTTATTTAGCAGAGG
TGGATAGGTACACATAAGAGATACATAATATCCTCTCTAGGGCTCTGTGT
ACTT
PON13UTR_eye_886925

(SEQ ID NO:119)

CCTGTGTCACTTTAGGTTCTTCTGTTAGAATGTTCTGGATTCGAAGCACC
TGTGGAAGAAATAACATTGGGTTAATATTTTTGTTAAGTCACTTAAAACA
ACCTTATGCATAATAGGGTCATCCTCCATAAGAAGCCATATAATCTTATA
CAATGAGATAAATCCTATTTTGTTCCAAAATTACACTTAACAC[A/C]CC
YGTTTCTTCATTTGGGAACAGGTGGGTACTGATCTCAAGTGATCTTTAGC
AGAGGTGGAGAGTATCTAGACCTACAGGGATGAGGCAGGAAATGCAAATG
AGCAGCGCATCCTGGATGTGGGACATTAGGGAGCAGTGGGGATTTCAGTA
AATG
CYP7PE1_580

(SEQ ID NO:120)

ATGTGTAAAAAGAACAAATAAACCTGTTTAAGATGGGCATAGCTAATAAA
TACATAAACTATAATCATTTAAAGAAAGATAAGAATGAGTTATTCATCAA
GCTTATAATTTGGTCTTGTTAACTTTTAAAGAAGAATTAATTTAGGTGCT
TTGCCAGAGAGACGGTGATCAAGTTCAGAGGAAAGAGAACTGGGAAAAAC
[A/G]TTTCTGCTGCTTCATGTTTCAGTGCTTTTATAAAATCATAGATAT
TTTACTACATATAGTAACATGTTACTCACCTAACATCCTCAAGAGAGTAT
TCTACATAAGTGCATCCTCTAGATAAATATAACTTATTGGAAGCATCAAA
ATTTTATCTATCATTCTTGTGACTAAATGTTTATCAATGTTTCTAA
GSTP1E2E4-5_419

(SEQ ID NO:121)

CCGGGCAAGGGGAGGGGGTGCTGGGCCTTAGGGGGCTGTGACTAGGATCG
GGGGACGCCCAAGCTCAGTGCCCCTCCCTGAGCCATGCCTCCCCCAACAG
CTATACGGGCAGCTCCCCAAGTTCCAGGACGGAGACCTCACCCTGTACCA
GTCCAATACCATCCTGCGTCACCTGGGCCGCACCCTTGGTGAGTCTTGAA
[A/C]CTCCAAGTCCAGGGCAGGCATGGGCAAGCCTCTGCCCCCGGAGCC
CTTTTGTTTAATCAGCTGCCCCGCAGCCCTCTGGAGTGNNNNNNNNNNNN
NNNNNNNNNNNNNNNNNNNNNNNNNNNNNNNNNNAGCCTGGGTGCCTGCA
ATCCTTGCCCTGTGCCAGGCTGCCTCCCAGGTGTCAGGTGAGCTCTGAGC
ACCTG
CYP2D6_RS2267446

(SEQ ID NO:122)

TGTGCGGGAGGACGGGGACAGCATTCAGCACCTACACCAGACAGAACGGG
GTCTCAATCC[C/T]TCCTGTGCTCTGCGTTCATCTGGACCAGTCTCAGG
CCCCAGCCATCTCCAGGAAGACCCA
CYP2E1RS2480259

(SEQ ID NO:123)

TAACAATGGTATTGATGAGACACTGAGGGAGTTTCAGAGACACGACACTG
TTAACAGTGAGACTAGGGGAAGCGGGGAGGAGGAGGCTGTGGGCAAACGG
CCATCACAAGGCAGGACTCACCCTTCTCTGCCTATTAAAAGAAGCACAAA
ATGTACTTGAGTGGAAGGATACCAGCAACACTGACTAATTCGGGGACCAG
[T/C]CTTCATACAATCTCAATTATCCAGAGCTCAACTGCAAAAACCGGT
GTTAAGTTGGGAAGACCGTGTCTGCCCAGTAAAGAAGGCACCCCCAGTGC
TCATCAGTGATGCCTACAGGAGGGAAATAGTGCCCTGTTAGAAGCTGGGG
CTCCAGCACTGGGTGTGAAAAGTATGGAGTTGGTTGTATGATCTTCCCAC
AACAA
CYP2D6_RS2004511

(SEQ ID NO:124)

GACTCTGTCAACTAGTCCTGCGCCCGAGAAGCTCCACAGTACCCTCTCCG
ACCCCACAGCAGGGCGCAGTCACACCTCTCAGAGGCACCCACACTGCCCC
CTCTCCCTGCAGGCG[C/T]TGGGTCCTCCAACATTCTGGCAGGTCCTGG
TTTGTCTCCCCACTAGACGGGGGCTCTGGATGGACAGGCCAGCCCTGCCT
ATACTCTGGACCCCCCACCCAAGTGGGGACAGTCAGTGTGGTGGCATTGA
GGACTAGGTGGCCAGGGTTCCTAGAGTGGGCCCACCTGGCAGTAGCCATG
CTGGGGCTATCACCAGGGGCTGGTGCTGAGCTGGGGTGAGGAGGGCGCCA
GGCCTACCTTAGGGATGCGGAAGCCCTGTACTTCGATGTCACGGGATGTC
ATATGGGTCACACCCAGGGGGACGATGTCCCCAAAGCGCTGCACCTCATG
AATCACGGCAGTGGTGTAGGGCATGTGAGCCTGGTCACCCATCTCTGGTC
GCCGCACCTGCCCTATCACGTCGTCGATCTCCTGTTGGACACGGCCTGGA
CAGACATGCGTCCCCACAATGGGTCAGCACCCAGGGGGTCCGGCCCTGAC
ACTCCTTCTTGCCTCCTATGTTG

### EXAMPLE 3

### Polymorphisms Associated with Response to Treatment with an Ace Inhibitor

A number of alleles of the polymorphic CYP2D6 cytochrome P450 isoenzyme 2D6 gene have previously been reported that affect drug metabolism. Individuals carrying these alleles often display a ‘poor metabolizer phenotype’ (PM) characterized by deficient hydroxylation of several classes of commonly used drugs, environmental toxic chemicals, and endogenous substances. CYP2D6 mutants have been correlated to drug overdosage and therapeutic failure because of poor metabolization of the prodrug to the active metabolite. For further discussion of the poor metabolizer phenotype/genotype, see Thompson et al., JAMA 289:1681-1690 (2003); Mukae et al., J Hum Hypertens. 16:857-63 (2002); Packard et al., Ann Pharmacother. 36:1058-67 (2002); Lee et al., Hypertension. 38:166-70 (2001).

In this example, the effects of various SNPs, haplotypes and SNP alleles, including the CYP2D6*4 (SEQ ID NO: 148) poor metabolizer allele, were studied as they affect ACE inhibitor responses. The strongest associations were found in the CYP2D6 gene. For markers 179 and 172, the TT and TC or a TC and TC genotype, respectively, were found at a higher frequency in myalgia case samples than normal controls (i.e., patients who took atorvastatin and were not removed from therapy due to myalgia) (Table 14).

| Polymorphism | Marker number |
|---|---|
| CYP2D6_RS1058174 | 179 |
| CYP2D6_RS2267446 | 172 |

TABLE 14
CYP2D6 179-172 RISK GENOTYPE: PATIENTS
WITH TT TC or TC TC GENOTYPE PAIR

| | WITH | WITHOUT | TOTAL | % WITH |
|---|---|---|---|---|
| DISC CASE | 34 | 16 | 50 | 0.68 |
| DISC CONTROL | 1 | 24 | 25 | 0.04 |

P < 0.0001

This genotype association was measured to be highly significant using a Fishers Exact test (two tailed) (p<0.0001) (Table 14).

To assess whether the TT-TC and TC-TC genotypes at 179-172 were linked to CYP2D6 poor metabolizer genotypes, each case and control was typed using a commercially available assay (DrugMEt™ Microarray Test and CYP2D6 Deletion/Duplication PCR Assay, manufactured by Jurilab of Kuopio Finland). CYP2D6 genotypes are determined using this assay as:

1—wild type

2—mutant, normal function

3—variant, poor metabolizer

4—variant, poor metabolizer

5—variant, poor metabolizer

6—variant, poor metabolizer

7—variant, poor metabolizer

8—variant, poor metabolizer

9—variant, poor metabolizer

10—variant, poor metabolizer

11—variant, poor metabolizer

12—variant, poor metabolizer

17—variant, poor metabolizer

As used herein, the term “poor metabolizer” refers to the response of patients with certain CYP2D6 variants to respond aberrantly to particular drugs (e.g. competitors or known substrates). The drugs that may be affected by poor metabolizer genotype and phenotype many that have been reported in the literature. However, the poor metabolizer also encompasses the responses to other drugs that have not yet been characterized as affected by the phenotype.

Atorvastatin patients were genotyped for the CYP2D6 gene. Thirty five patients were found to possess the CYP2D6*4 poor metabolizer allele, and each of these also possessed the CYP2D6 179-172 TT-TC or TC-TC genotype pair. The linkage between these genotypes was complete; each of the patients possessing the CYP2D6 179-172 TT-TC or TC-TC genotype pair was found to also have the CYP2D6*4 allele and all patients harboring the CYP2D6*4 allele also had the CYP2D6 179-172 TT-TC or TC-TC genotype pair.

Table 15, below, shows the data of Table A expressed in terms of CYP2D6*4 genotypes.

TABLE 15
CYP2D6 179-172 RISK GENOTYPE: PATIENTS
WITH CYP2D6*4 Poor Metabolizer ALLELE

| | WITH | WITHOUT | TOTAL | % WITH |
|---|---|---|---|---|
| DISC CASE | 34 | 16 | 50 | 0.68 |
| DISC CONTROL | 1 | 24 | 25 | 0.04 |

P < 0.0001

The frequency of the *4 allele in the Caucasian population has been reported to be about 15% (Stamer, et al. Clin Chem 48:1412-7 (2002); Menoyo, et al., Cell Biochem Funct. Aug. 30m 2005, Epub ahead of print), and this frequency is more similar to the 4% frequency observed in the Discovery Controls (normal atorvastatin response in terms of no myalgia) than for the Discovery Case samples (68%).

These results indicated that the CYP2D6 SNP associations described above for the Discovery set, were part of a CYP2D6 haplotype known to be of functional relevance for drug metabolism. This finding lends provides further support that the claimed SNPs are involved in muscle adverse reactions and provides additional validation because generic poor metabolizer alleles of the CYP2D6 gene have previously been documented by others. Furthermore, if the identified SNP were not spurious, it could be expected that the alleles for the SNPs were linked with one or more poor metabolizer variants.

More specifically, these studies showed an association between myalgia and one particular CYP2D6 poor metabolizer allele, the CYP2D6*4) rather than a more generalized “poor metabolizer” status. For example, no associations were found between myalgia response and CYP2D6 poor metabolizer alleles other than the CYP2D6*4 allele.

A. Blind Validation

Showing that the variant CYP2D6 179-172 alleles associated with atorvastatin-induced myalgia are linked to the well-known CYP2D6*4 poor metabolizer allele constitutes one form of validation for these SNPs. Further validation can be shown by demonstrating that the associations extend to an independent sample set.

For these studies, a set of 64 atorvastatin patients (“Blind Cases and Controls”) were tested: 15 of the patients had been withdrawn from the drug due to myalgia (“Blind Cases”) while 49 had not been removed from the drug due to myalgia (“Blind Controls”, normal response). The Jurilab DrugMEt™ assay was used to genotype patients. As summarized below in Table 16, the association between CYP2D6*4 (equivalent to the CYP2D6 179-172 TT-TC or TC-TC genotype pair) and myalgia response was confirmed in the new blind trial patients.

TABLE 16
CYP2D6 GENOTYPE: PATIENTS WITH CYP2D6*4 allele

| | WITH | WITHOUT | TOTAL | % WITH |
|---|---|---|---|---|
| BLIND CASE | 9 | 7 | 15 | 0.6 |
| BLIND CONTROL | 12 | 37 | 49 | 0.24 |

P = 0.0300

The frequency of the risk CYP2D6*4 allele in the Blind Case set (60%; Table C) was similar to that originally observed in the Discovery Case set (68%, Table B). This result demonstrates that the association between the CYP2D6 marker 179-172 alleles, which are part of the well-known CYP2D6*4 haplotype, are indeed associated with atorvastatin induced myalgia. The combined results of the Discovery and Blind Case/Control samples are given below in Table D:

TABLE 17
CYP2D6 *4 ALLELE PRESENCE IN ATORVASTATIN (LIPITOR) PATIENTS WHO
WERE REMOVED FROM THERAPY DUE TO MYALGIA (CASE) VERSUS THOSE
WHO WERE NOT (CONTROL)

| SAMPLE SET | STATUS | WITH | WITHOUT | TOTAL | FREQ. | P VALUE |
|---|---|---|---|---|---|---|
| DISCOVERY | CASE | 24 | 27 | 51 | 0.47059 | |
| DISCOVERY | CONTROL | 11 | 44 | 55 | 0.2 | P = 0.0039 |
| BLIND | CASE | 10 | 8 | 18 | 0.55556 | |
| BLIND | CONTROL | 2 | 15 | 17 | 0.11765 | P = 0.0116 |
| TOTAL | CASE | 34 | 35 | 69 | 0.49275 | |
| TOTAL | CONTROL | 13 | 59 | 72 | 0.18056 | P = 0.0001 |

As shown in Table 17, the observed frequency of the CYP2D6*4 allele in the Combined (Total) Control sample is 18%, about the same in the general Caucasian population of about 15%-18% (depending on the ethnicity). However, the frequency in the Combined (Total) Case sample is much higher (˜50%). Given the sample size involved, this result is highly significant (Fishers Exact Test, two-tailed p<0.0001).

Overall, and in summary, these results clearly show:
1. The claimed CYP2D6 marker 179-172 alleles are linked completely with and therefore part of the well-known CYP2D6*4 haplotype.
2. That the claimed CYP2D6 marker 179-172 alleles equivalent to the well-known CYP2D6*4 haplotype were highly associated with atorvastatin-induced myalgia in a first set of 75 samples.
3. That the claimed CYP2D6 marker 179-172 alleles equivalent to the well-known CYP2D6*4 haplotype were also highly associated with atorvastatin induced myalgia in a second set of 64 validation samples.
4. Therefore, the claimed CYP2D6 marker 179-172 alleles equivalent to the well-known CYP2D6*4 haplotype constitute a validated risk factor for atorvastatin induced myalgia.

B. Other SNPs Associated with the Risk CYP2D6 Alleles

Additional SNPs were also determined in the atorvastatin patients. The genotypes of another SNP were found to be in Linkage Disequilibrium with the CYP2D6 179-172 TT-TC or TC-TC genotype pair, which as we have shown is linked with the CYP2D6*4 Poor Metabolizer allele.

| Polymorphism | Marker number |
|---|---|
| CYP2D6_RS2267444 | 93 |

In particular, the “C” genotype was present for 28 of the discovery samples, and 23 of these also had the 179-172 TC-TC or TT-TC genotype pair.

1. Other CYP2D6 Alleles Associated with Atorvastatin Response

Using the Jurilab DragMEt™ assay to genotype the samples we also noted that the CYP2D6*10 allele was associated with response, but unlike the *4 allele this allele was associated with normal (non-myalgia) response (Table 185:

TABLE 18

| | No. CASE | No. CONTROL |
|---|---|---|
| Sample Set | with CYP2D6*10 | with CYP2D6*10 |
| Discovery Samples | 1 | 6 |
| BLIND Test Samples | 0 | 2 |

As shown in Table 18, of the 7 samples from the Discovery set that had the *10 allele, 6 of were from normal responders. Two of the Blind test samples had the *10 allele, and both of these patients were normal responders. In total, 8/9 individuals who had the 10 allele were normal responders. The *10 allele was found to be present in each patient that had the CYP2D6 179-172 CC-TC genotype pair—each of the patients with the CYP2D6 179-172 CC-TC genotype pair was found to be either heterozygous or homozygous for the CYP2D6*10 allele. Thus, the CYP2D6 179-172 CC-TC genotype pair is a marker for the CYP2D6*10 allele, which is associated with normal (non-myalgia) response to atorvastatin.

C. CYP2C8 Haplotypes Associated with Risk of Atorvastatin Induced Myalgia

The atorvastatin studies also found that the following alleles of the CYP2C8 gene are associated with atorvastatin-induced myalgia, though to a lesser extent than the CYP2D6 179-172 TT-TC or TC-TC genotype pair/CYP2D6*4 allele:

| Marker | SNP Name |
|---|---|
| 391 | CYP2C8_RS1934952 |
| 374 | CYP2C8_RS1934953 |
| 369 | CYP2C8_RS1891071 |
| 225 | CYP2C8_RS1891073 |
| 362 | CYP2C8_2071426 |
| 249 | CYP2C8_RS1891072 |
| 2760 | CYP2C8_2185571 |
| 282 | CYP2C8E8_296 (rs1934951) |
| 54 | CYP2C8E7-5_349 (RS2275620) |
| 134 | CYP2C8E2E3_397 (RS2275622) |

The phase-known sequence of alleles for these SNPs, along the chromosome in order shown above, constitute haplotypes. Three of these haplotypes were associated with the myalgia adverse event—that is, the haplotype alleles were present at a higher frequency in individuals who had been withdrawn from atorvastatin due to myalgia (CASE) compared to normal patients that had not (CONTROL).

| | | |
|---|---|---|
| SEQ ID NO'S 149-152 | CASE | CONTROL |

| | | |
|---|---|---|
| Haplotype 1: AAATGTTGAC | 34 | 11 |
| Haplotype 2: AAGTATCGTC | 3 | 0 |
| Haplotype 5: GAATATCGAC | 16 | 6 |
| Haplotype 6: GAATATCAAC | 19 | 17 |

Diploid pairs of these haplotypes were distributed as follows:

| Genotype | CASE | CONTROL |
|---|---|---|
| 1, 1 | 8 | 1 |
| 1, 5 | 6 | 2 |
| 6, 6 | 1 | 6 |

In addition, Native American and East Asian admixture was associated with myalgia and normal response:

| Ancestry | CASE | CONTROL |
|---|---|---|
| European, with >11% Native American admixture | 4 | 0 |
| European, with >11% East Asian admixture | 1 | 5 |

D. Classification Accuracy within Discovery Set

The results described above demonstrate that alleles of two separate Cytochrome P450 genes are associated with atorvastatin-induced myalgia response. The following conclusion can be drawn from the data presented above:
1) The CYP2D6 gene, where the CYP2D6*4 allele marked by the CYP2D6 179-172 TT-TC or TC-TC genotype pair, or directly with a CYP2D6*4 genotyping assay, is indicative of likelihood to express atorvastatin induced myalgia
2) The CYP2D6*10 allele marked by the 179-172 CC-TC genotype pair, or directly with a CYP2D6*10 genotyping assay, is indicative of likelihood to express normal (non-myalgia) response to atorvastatin.
3) Certain CYP2C8 haplotypes as discussed above, are indicative for myalgia or normal response to atorvastatin.
4) Native American and East Asian admixture as discussed above indicative for myalgia or normal response to atorvastatin.

A simple classification can be used to combine the CYP2D6 and CYP2C8 risk alleles into a scheme, where possession of a “risk allele” or “factor” can be used to infer the classification as a myalgia responde, and possession of a “protective allele” or “factor” can be used to infer the classification as a normal responder, and lack of a “risk” or “protective” allele or factor bestows upon the individual an inconclusive classification. This scheme partitions the Discovery set of atorvastatin patients in the following way (Table 19):

TABLE 19

| | classified | | classified |
|---|---|---|---|
| | MYALGIA | incon | NORM |
| actual MYALGIA | 35 | 15 | 1 |
| actual NORM | 1 | 14 | 22 |

As shown in Table 19, approximately ⅔rds of the patients carried a risk or protective allele and could therefore be classified; of these, 97% were correctly classified using the above classification scheme.

Although the invention has been described with reference to the above examples, it will be understood that modifications and variations are encompassed within the spirit and scope of the invention. Accordingly, the invention is limited only by the following claims.

What is claimed is:

1. A method for inferring a muscle adverse effect statin response of a human subject from a nucleic acid sample of the subject, comprising identifying in the nucleic acid sample, an occurrence of at least one statin response-related single nucleotide polymorphism (SNP) of a marker, wherein the marker has a sequence selected from the group consisting of:
a. nucleotides 3911-4379 of SEQ ID NO: 134;
b. nucleotides 31264-31822 of SEQ ID NO: 131;
c. nucleotides 22281-23778 of SEQ ID NO: 143;
d. nucleotides 3482-4414 of SEQ ID NO: 134;
e. nucleotides 1895-2286 of SEQ ID NO: 132;
f. nucleotides 650-1166 of SEQ ID NO:131;
g. nucleotides 771-1171 of SEQ ID NO:142;
h. nucleotides 79999-80360 of SEQ ID NO: 133;
i. nucleotides 31264-31822 of SEQ ID NO: 131;
j. nucleotides 2440-2560 of SEQ ID NO: 134;
k. nucleotides 23757-24069 of SEQ ID NO: 131;
l. nucleotides 30438-30711 of SEQ ID NO: 131;
m. nucleotides 23571-24967 of SEQ ID NO: 131;
n. nucleotides 12971-14510 of SEQ ID NO: 143;
o. nucleotides 26896-27098 of SEQ ID NO: 130;
p. nucleotides 8115-8737 of SEQ ID NO: 143;
q. nucleotides 13465-13865 of SEQ ID NO:138;
r. nucleotides 26056-26456 of SEQ ID NO: 138;
s. nucleotides 26167-26197 of SEQ ID NO:138;
t. nucleotides 17636-18035 of SEQ ID NO: 138;
u. nucleotides 25354-25754 of SEQ ID NO: 138;
v. nucleotides 12153-12553 of SEQ ID NO:138;
w. nucleotides 7082-7942 of SEQ ID NO: 139;
x. nucleotides 5779-5827 of SEQ ID NO:135;
y. nucleotides 5851-6442 of SEQ ID NO:135;
z. nucleotides 7909-8504 of SEQ ID NO:139;
aa. nucleotides 651-1166 of SEQ ID NO:131;
ab. nucleotides 4351-4750 of SEQ ID NO: 139;
ac. nucleotides 3138-3500 of SEQ ID NO:139;
ad. nucleotides 3482-4414 of SEQ ID NO:131;
ae. nucleotides 4397-4797 of SEQ ID NO:139;
af. nucleotides 31264-31813 of SEQ ID NO:131;
ag. nucleotides 16240-16589 of SEQ ID NO:145;
ah. nucleotides 25192-2298 of SEQ ID NO:145;
ai. nucleotides 11344-12528 of SEQ ID NO: 139;
aj. nucleotides 2800-3685 of SEQ ID NO:129;
ak. nucleotides 30350-30631 of SEQ ID NO: 131;
al. nucleotides 750-1110 of SEQ ID NO:134;
am. nucleotides 5880-6229 of SEQ ID NO:145;
an. nucleotides 25192-25479 of SEQ ID NO: 145;
ao. nucleotides 17794-18106 of SEQ ID NO:130;
ap. nucleotides 26895-27098 of SEQ ID NO:130;
aq. nucleotides 26895-25478 of SEQ ID NO: 130;
ar. nucleotides 34-825 of SEQ ID NO:127;
as. nucleotides 11012-11412 of SEQ ID NO: 135;
at. nucleotides 3178-3786 of SEQ ID NO:134;
au. nucleotides 143-518 of SEQ ID NO:140;
av. nucleotides 17795-18116 of SEQ ID NO: 130;
aw. nucleotides 3388-3786 of SEQ ID NO:134;
ax. nucleotides 502-902 of SEQ ID NO:126;
ay. nucleotides 23737-24368 of SEQ ID NO:131;
az. nucleotides 1805-2204 of SEQ ID NO:131;
ba. nucleotides 5841-6441 of SEQ ID NO: 135;
bb. nucleotides 26613-27098 of SEQ ID NO:130;
bc. nucleotides 19968-20369 of SEQ ID NO: 138;
bd. nucleotides 19636-21357 of SEQ ID NO:136;
be. nucleotides 5881-6229 of SEQ ID NO:142; and
bf. nucleotides 2440-2560 of SEQ ID NO:134;
or the complement of any of a-bf above;
wherein the SNP is associated with a muscle adverse effect in response to administration of the statin, thereby inferring the muscle adverse effect statin response of the subject.

2. The method of claim 1, wherein the SNP is located at:
a. nucleotide 4332 of SEQ ID NO: 134;
b. nucleotides 31683 of SEQ ID NO: 131;
c. nucleotides 23077 of SEQ ID NO: 143;
d. nucleotides 4208 of SEQ ID NO: 134;
e. nucleotides of SEQ ID NO: 132;
f. nucleotides 860 of SEQ ID NO:131;
g. nucleotides 971 of SEQ ID NO:142;
h. nucleotides 2098 of SEQ ID NO: 133;
i. nucleotides 80160 SEQ ID NO: 131;
j. nucleotides 2500 of SEQ ID NO: 134;
k. nucleotides 23809 of SEQ ID NO: 131;
l. nucleotides 30635 of SEQ ID NO: 131;
m. nucleotides 24272 of SEQ ID NO: 131;
n. nucleotides 13780 of SEQ ID NO: 143;
o. nucleotides 296935 of SEQ ID NO: 130;
p. nucleotides 8462 of SEQ ID NO: 143;
q. nucleotide 13665 of SEQ ID NO: 138;
r. nucleotide 26256 of SEQ ID NO: 138;
s. nucleotide 26137 of SEQ ID NO:138;
t. nucleotide 17836 of SEQ ID NO:138;
u. nucleotide 25554 of SEQ ID NO: 138;
v. nucleotide 12353 of SEQ ID NO:138;
w. nucleotide 7444 of SEQ ID NO:139;
x. nucleotide 5832 of SEQ ID NO:135;
y. nucleotide 6063 of SEQ ID NO:135;
z. nucleotide 8004 of SEQ ID NO:139;
aa. nucleotide 860 of SEQ ID NO:131;
ab. nucleotide 4550 of SEQ ID NO: 139;
ac. nucleotide 3300 of SEQ ID NO:139;
ad. nucleotide 4208 of SEQ ID NO: 131;
ae. nucleotide 4597 of SEQ ID NO:139;
af. nucleotide 31671 of SEQ ID NO: 131;
ag. nucleotide 16399 of SEQ ID NO:145;
ah. nucleotide 2097 of SEQ ID NO:145;
ai. nucleotide 11987 of SEQ ID NO:139;
aj. nucleotide 3500 of SEQ ID NO:129;
ak. nucleotide 30434 of SEQ ID NO:131;
al. nucleotide 930 of SEQ ID NO:134;
am. nucleotide 6046 of SEQ ID NO: 145;
an. nucleotide 25286 of SEQ ID NO:145;
ao. nucleotide 18060 of SEQ ID NO:130;
ap. nucleotide 26950 of SEQ ID NO:130;
aq. nucleotide 26950 of SEQ ID NO:130;
ar. nucleotide 734 of SEQ ID NO:127;
as. nucleotide 11212 of SEQ ID NO:135;
at. nucleotide 3671 of SEQ ID NO: 134;
au. nucleotide 326 of SEQ ID NO: 140;
av. nucleotide 18060 of SEQ ID NO: 130;
aw. nucleotide 3671 of SEQ ID NO:134;
ax. nucleotide 702 of SEQ ID NO:126;
ay. nucleotide 24205 of SEQ ID NO:131;
az. nucleotide 2005 of SEQ ID NO:131;
ba. nucleotide 6063 of SEQ ID NO:135;
bb. nucleotide 26806 of SEQ ID NO:130;
bc. nucleotide 20169 of SEQ ID NO:138;
bd. nucleotide 20343 of SEQ ID NO:136;
be. nucleotide 6183 of SEQ ID NO:142; or
bf. nucleotide 2500 of SEQ ID NO:134.

3. The method of claim 1, comprising identifying a nucleotide occurrence of each of at least two statin response-related SNPs.

4. The method of claim 1, wherein identifying a nucleotide occurrence of at least one statin response-related SNP comprises
a) incubating the nucleic acid sample with a probe or primer that selectively hybridizes to or near a nucleic acid molecule comprising the nucleotide occurrence of the SNP, and
b) detecting selective hybridization of the primer or probe, thereby identifying the nucleotide occurrence.

5. The method of claim 4, wherein detecting selective hybridization of the primer comprises performing a primer extension reaction, and detecting a primer extension reaction product comprising the primer.

6. The method of claim 5, wherein the primer extension reaction comprises a polymerase chain reaction.

7. The method of claim 1, comprising identifying a nucleotide occurrence of each of at least two statin response-related SNPs.

8. The method of claim 1, wherein the statin is Lipitor®.

9. The method of claim 1, wherein the statin is Zocor®.

10. A method for inferring a muscle adverse effect statin response of a human subject from a nucleic acid sample of the subject, the method comprising identifying in the nucleic acid sample, an occurrence of at least one statin response-related single nucleotide polymorphism (SNP) of a marker as set forth in Table 2 and 5, wherein the SNP is associated with a muscle adverse effect in response to administration of the statin, thereby inferring the muscle adverse effect statin response of the subject.

11. The method of claim 10, wherein the marker has a nucleotide sequence selected from the group consisting of: SEQ ID NOs 1-27 and 46-73.

12. A method for inferring a dry cough adverse effect angiotensin converting enzyme (ACE) inhibitor response of a human subject from a nucleic acid sample of the subject, the method comprising identifying, in the nucleic acid sample, a nucleotide occurrence of at least one ACE inhibitor response-related single nucleotide polymorphism (SNP) of a marker as set forth in any of Tables 1, 2, 4, 5, wherein the nucleotide occurrence is associated with a dry cough effect in response to administration of the ACE inhibitor, thereby inferring the dry cough adverse effect ACE inhibitor response of the subject.

13. The method of claim 12, wherein identifying a nucleotide occurrence of at least one ACE inhibitor response-related SNP comprises
a) incubating the nucleic acid sample with a probe or primer that selectively hybridizes to or near a nucleic acid molecule comprising the nucleotide occurrence of the SNP, and
b) detecting selective hybridization of the primer or probe, thereby identifying the nucleotide occurrence.

14. The method of claim 13, wherein detecting selective hybridization of the primer comprises performing a primer extension reaction, and detecting a primer extension reaction product comprising the primer.

15. The method of claim 14, wherein the primer extension reaction comprises a polymerase chain reaction.

16. The method of claim 12, comprising identifying a nucleotide occurrence of each of at least two ACE inhibitor response-related SNPs.

17. The method of claim 12, wherein the ACE is enalapril.

18. The method of claim 9, wherein the ACE inhibitor is lisinopril.

19. A method for inferring a poor metabolizer phenotype of a human subject from a nucleic acid sample of the subject, comprising identifying in the nucleic acid sample, an occurrence of at least one single nucleotide polymorphism (SNP) of a CYP2D6 marker, wherein the SNP is associated with the poor metabolizer phenotype, thereby inferring the poor metabolizer phenotype of the subject.

20. The method of claim 19, wherein the CYP2D6 marker comprises at least about 100 nucleotides of SEQ ID NO: 134, 147 or 148.

21. The method of claim 19, wherein the marker is nucleotides 3911-4379 of SEQ ID NO: 134.

22. The method of claim 21, wherein the SNP is located at nucleotide 4332 of SEQ ID NO: 134.

23. The method of claim 19, wherein the marker is nucleotides 2440-2560 of SEQ ID NO: 134.

24. The method of claim 23, wherein the SNP is located at nucleotide 2500 of SEQ ID NO:134.

25. The method of claim 19, wherein the marker is CYP2D6_RS1058174.

26. The method of claim 19, wherein the marker is CYP2D6_RS2267446.

27. The method of claim 19, comprising identifying a SNP at nucleotide 4332 of SEQ ID NO: 134 and a SNP at nucleotide 2500 of SEQ ID NO: 134.

28. The method of claim 27, comprising identifying the genotype of the SNPs at nucleotide 4332 of SEQ ID NO:134 and nucleotide 2500 of SEQ ID NO:134, wherein a TT and TC genotype or a TC and TC genotype, respectively, is associated with a poor metabolizer phenotype.

29. The method of claim 19, wherein a poor metabolizer phenotype is associated with myalgia in atorvastatin-treated patients, thereby inferring myalgia response to atorvostatin.

30. The method of claim 19, wherein the poor metabolizer phenotype is associated with the CYP2D6*4 allele.
